(19) United States
(12) Patent Application Publication
(10) Pub. No.: US 2026/0213025 A1
(43) Pub. Date: Jul. 23, 2026

(54) ARTIFICIAL INTELLIGENCE BASED SYSTEM AND METHOD FOR AUTOMATICALLY DETERMINING BIOLOGICAL STATUS INFORMATION OF RESOURCES USING MULTIANALYTE DATABASE

(71) Applicant: Convergent Animal Health, LLC, Overland Park, KS (US)

(72) Inventors: Nedal R. Saleh, San Jose, CA (US); Richard A. Vierling, JR., Ste. Genevieve, MO (US)

(21) Appl. No.: 19/457,241

(22) Filed: Jan. 23, 2026

Related U.S. Application Data
(63) Continuation-in-part of application No. 19/027,032, filed on Jan. 17, 2025, now abandoned.

(51) Int. Cl. G16H 70/60

(52) U.S. Cl. CPC G16H 70/60

(57) ABSTRACT

AI-based systems and methods for automatically determining biological status information of resources based on pathogens using multianalyte database, are disclosed. The AI-based method comprises analyzing obtained analyte data comprising analytes, to determine biological status information indicative of response of resources to each pathogen using AI model, by: (a) extracting receptor binding characteristics from obtained analyte data, (b) determining host cell adherence capability from obtained analyte data, (c) identifying virulence genetic factors comprising virulence gene sequences, toxin-encoding genetic elements, immune evasion markers, pathogenicity indicators, in obtained analyte data, (d) classifying pathogens comprising potential zoonotic pathogens from the obtained analyte data, (e) assigning pandemic threat scores to potential zoonotic pathogens based on the receptor binding characteristics, host cell adherence capability, and virulence genetic factors, and (f) determining the biological status information, based on the pandemic threat scores assigned to the potential zoonotic pathogens, which all implemented using AI model.

### CROSS REFERENCE TO RELATED APPLICATION(S)

This application claims the priority to incorporates by reference the entire disclosure of U.S. non-provisional patent application Ser. No. 19/027,032 , filed on Jan. 17, 2025, titled “ARTIFICIAL INTELLIGENCE BASED (AI-BASED) VERTICALLY INTEGRATED PATHOGENICACTIVE SURVEILLANCE SYSTEM AND METHOD FOR AUTOMATICALLY MONITORINGPATHOGENS USING MULTIANALYTE DATABASE”.

### TECHNICAL FIELD

Embodiments of the present disclosure relate to artificial intelligence based (AI-based) surveillance systems and methods, and more particularly relate to AI-based systems and methods for automatically determining biological status information of one or more resources and also monitoring one or more pathogens using multianalyte database.

### BACKGROUND

Surveillance is a form of monitoring that involves taking action based on the analysis of gathered information. An ideal surveillance strategy may be utilized to predict a disease outbreak before the outbreak occurs so preventive measures may be implemented. Unfortunately, current surveillance systems are not predictive and depend on a disease event occurring. The steps are: (a) occurring of disease events, (b) recognizing the disease events by reporting resources, (c) reporting the disease events to a public health agency, (d) controlling and preventing activities, and (e) disseminating information associated with the disease events to the public. This passive approach depends on people to provide quality data and timeliness, which may be issues.

Pathogenic surveillance is critical for detecting, monitoring, and controlling infectious diseases in humans, livestock, wildlife, pets, and the environment. Traditional approaches to pathogen detection have often been limited in scope, focusing on individual samples, species, or environments, without fully integrating diverse sources of data. This fragmented approach makes it challenging to identify early signs of emerging diseases or cross-species pathogen transmission. Additionally, existing surveillance systems often rely on conventional analysis of biomarkers or molecular detection methods, which may not be sensitive or comprehensive enough for large-scale surveillance of multiple pathogens across multiple ecosystems.

MicroRNA (miRNA)-based tests have emerged as a powerful tool for detecting specific biological responses to pathogens and environmental agents, the 2024 Nobel Prize in Physiology or Medicine was granted for the discovery of microRNA. MicroRNAs are small, non-coding RNA molecules that regulate gene expression and are highly responsive to biological changes, including infection by pathogens and environmental agents. The miRNA-based tests offer the advantage of high sensitivity and specificity for detecting pathogens at various stages of infection, and their expression profiles may provide valuable insights into the biological status of an organism. In addition to miRNA tests, additional tests may be performed to detect pathogens based on other analytes including at least one of: nucleic acid data, deoxyribonucleic acid (DNA), ribonucleic acid (RNA), messenger RNA (mRNA), and microRNA (miRNA), protein concentration, protein three dimensional (3D) structures, protein-protein interactions, ligand receptor binding, carbohydrates, disease state, molecules, metadata, and the like.

The emergence of miRNA-based tests has proven effective in laboratory settings and for specific applications, there is a need to expand these tests into larger systems. This expansion of the miRNA-based tests in addition to other analyte tests requires AI-based multianalyte data analysis of the data in the surveillance system, which is never configured in current approaches.

Hence, there is a need in the art for an AI-based system and method for automatically determining biological status information of one or more resources by monitoring one or more pathogens using multianalyte database, in order to address at least the aforementioned issues.

### SUMMARY

This summary is provided to introduce a selection of concepts, in a simple manner, which is further described in the detailed description of the disclosure. This summary is neither intended to identify key nor essential inventive concepts of the subject matter nor to determine the scope of the disclosure.

An aspect of the present disclosure provides an artificial intelligence (AI) based method for automatically determining biological status information of one or more resources based on one or more pathogens using multianalyte database. The AI-based method comprises obtaining, by one or more hardware processors, analyte data from one or more samples collected from the one or more resources. The one or more samples are collected from one or more resources comprising at least one of: environments, humans and animals.

The analyte data are obtained from the one or more samples through at least one of: one or more sensors, one or more detectors, manual and automated data collectors, and laboratory analysis. The analyte data comprises information associated with one or more analytes obtained from the one or more resources via one or more samples of the one or more resources.

The AI-based method further comprises storing, by the one or more hardware processors, the analyte data associated with the one or more analytes, in the multianalyte database. The multianalyte database comprises pre-stored analyte data associated with the one or more pre-stored analytes in one or more pre-defined patterns. Each of the one or more pre-defined patterns is one or more combinations of the one or more pre-stored analytes, indicative of the one or more pathogens, historical analyte data from known pathogen exposures, and one or more reference patterns for pathogen identification.

The AI-based method further comprises comparing, by the one or more hardware processors, the obtained analyte data analyzing, by the one or more hardware processors, the obtained analyte data comprising the one or more analytes, to determine the biological status information indicative of a response of the one or more resources to each of the one or more pathogens using the AI model.

Analyzing the obtained analyte data comprises ingesting, by the one or more hardware processors, the obtained analyte data comprising the one or more analytes at the AI model. In an embodiment, the one or more analytes comprise at least one of: nucleic acid data, deoxyribonucleic acid (DNA), ribonucleic acid (RNA), messenger RNA (mRNA), and microRNA (miRNA), protein concentration, protein three dimensional (3D) structures, protein-protein interactions, ligand receptor binding, protein-protein interactions, ligand receptor binding, carbohydrates, disease state, molecules, and metadata. The metadata comprise at least one of: one or more species, one or more locations where the one or more samples are collected from the one or more resources, and one or more dates on which the one or more samples are collected.

Analyzing the obtained analyte data further comprises retrieving, by the one or more hardware processors, the pre-stored analyte data from the multianalyte database. Analyzing the obtained analyte data further comprises comparing, by the one or more hardware processors, the obtained analyte data with the pre-stored analyte data, to identify the one or more pathogens, using the AI model. Analyzing the obtained analyte data further comprises correlating, by the one or more hardware processors, one or more analyte patterns in the obtained analyte data, with known pathogen signatures, to determine pathogen-specific signatures within the obtained analyte data, using the AI model.

Analyzing the obtained analyte data further comprises extracting, by the one or more hardware processors, receptor binding characteristics from the obtained analyte data, using the AI model, by: (a) identifying, by the one or more hardware processors, receptor binding proteins and concentrations of the receptor binding proteins, within the obtained analyte data; (b) analyzing, by the one or more hardware processors, three dimensional protein structures in the obtained analyte data for determining binding domain compatibility; (c) evaluating, by the one or more hardware processors, ligand receptor binding profiles present in the obtained analyte data; and (d) generating, by the one or more hardware processors, one or more scores for binding affinity potential based on molecular interaction data within the obtained analyte data.

Analyzing the obtained analyte data further comprises determining, by the one or more hardware processors, host cell adherence capability from the obtained analyte data, using the AI model, by: (a) detecting, by the one or more hardware processors, adhesion protein markers and concentrations in the obtained analyte data; (b) analyzing, by the one or more hardware processors, protein-protein interaction profiles within the obtained analyte data for cell surface binding; (c) identifying, by the one or more hardware processors, attachment mechanism indicators present in the obtained analyte data; and (d) quantifying, by the one or more hardware processors, potential host cell adherence using molecular signatures found in the obtained analyte data.

Analyzing the obtained analyte data further comprises identifying, by the one or more hardware processors, one or more virulence genetic factors comprising at least one of: virulence gene sequences, toxin-encoding genetic elements, immune evasion markers, pathogenicity indicators, in the obtained analyte data, using the AI model.

Analyzing the obtained analyte data further comprises classifying, by the one or more hardware processors, the one or more pathogens comprising potential zoonotic pathogens from the obtained analyte data, using the AI model, by: (a) comparing, by the one or more hardware processors, the pathogen-specific signatures within the obtained analyte data against known zoonotic pathogen patterns; (b) identifying, by the one or more hardware processors, species-jumping genetic markers within the obtained analyte data; (c) detecting, by the one or more hardware processors, cross-species transmission indicators present in the obtained analyte data; and (d) classifying, by the one or more hardware processors, the potential zoonotic pathogens based on one or more patterns associated with the potential zoonotic pathogens determined in the obtained analyte data.

Analyzing the obtained analyte data further comprises assigning, by the one or more hardware processors, one or more pandemic threat scores to the potential zoonotic pathogens based on at least one of: the receptor binding characteristics, the host cell adherence capability, and the one or more virulence genetic factors, using the AI model. Analyzing the obtained analyte data further comprises determining, by the one or more hardware processors, the biological status information indicative of a response of the one or more resources to each of the one or more pathogens comprising the potential zoonotic pathogens, based on the one or more pandemic threat scores assigned to the potential zoonotic pathogens, using the AI model.

The AI-based method further comprises providing, by the one or more hardware processors, the biological status information indicative of a response of the one or more resources to each of the one or more pathogens comprising the potential zoonotic pathogens, as an output to a display device associated with one or more users.

In an embodiment, comparing the obtained analyte data with the pre-stored analyte data, to identify the one or more pathogens, using the AI model, comprises: (a) matching, by the one or more hardware processors, one or more analyte patterns in the obtained analyte data against the one or more pre-defined patterns, associated with the one or more pathogens, in the pre-stored analyte data; (b) identifying, by the one or more hardware processors, one or more similarities and one or more differences, between current analyte profiles and historical analyte profiles; (c) determining, by the one or more hardware processors, correlation coefficients between the obtained analyte data and known pathogen signatures; and (d) determining, by the one or more hardware processors, one or more pattern matching scores for pathogen identification.

In another embodiment, correlating the one or more analyte patterns in the obtained analyte data, with the known pathogen signatures, to determine pathogen-specific signatures within the obtained analyte data, using the AI model, comprises: (a) mapping, by the one or more hardware processors, specific analyte combinations in the obtained analyte data to the known pathogen signatures; (b) identifying, by the one or more hardware processors, which of the one or more pathogens is potentially present based on analyte pattern matching; and; and (c) determining, by the one or more hardware processors, pathogen-specific signatures within the obtained analyte data comprising the one or more analytes.

In yet another embodiment, assigning the one or more pandemic threat scores to the potential zoonotic pathogens, using the AI model, comprises: (a) integrating, by the one or more hardware processors, the receptor binding characteristics, the host cell adherence capability, and the one or more virulence genetic factors using weighted algorithmic models within the AI model to compute composite risk metrics for each of the potential zoonotic pathogens based on the obtained analyte data; and (b) assigning, by the one or more hardware processors, the one or more pandemic threat scores to each of the potential zoonotic pathogens by applying multi-factor risk assessment algorithms within the AI model that combine composite risk metrics derived from the receptor binding characteristics, the host cell adherence capability, and the one or more virulence genetic factors, identified in the obtained analyte data.

In yet another embodiment, determining the biological status information, using the AI model, comprises: (a) correlating, by the one or more hardware processors, the one or more pandemic threat scores assigned to the potential zoonotic pathogens with resource-specific response patterns stored in the multianalyte database, wherein the AI model matches high pandemic threat scores to corresponding biological response indicators for each of the one or more resources; (b) analyzing, by the one or more hardware processors, resource-specific vulnerability factors using the AI model by processing the obtained analyte data to identify immune system markers, stress response proteins, and inflammatory indicators specific to each of the one or more resources in response to the potential zoonotic pathogens with assigned pandemic threat scores; (c) generating, by the one or more hardware processors, pathogen-specific biological impact assessments using the AI model by combining the one or more pandemic threat scores with resource response data from the obtained analyte data to determine infection severity, progression rates, and recovery potential for each of the potential zoonotic pathogens affecting each of the one or more resources; (d) determining, by the one or more hardware processors, resource response severity levels using the AI model by weighting the one or more pandemic threat scores against resource-specific susceptibility indicators identified in the obtained analyte data, wherein higher pandemic threat scores correlate to severe biological status information for vulnerable resources; and (e) determining, by the one or more hardware processors, the biological status information indicative of the response of the one or more resources to each of the one or more pathogens comprising the potential zoonotic pathogens by integrating the pathogen-specific biological impact assessments and the resource response severity levels through the AI model to generate comprehensive biological status information that reflect the pandemic threat level of each potential zoonotic pathogen on each of the one or more resources.

In yet another embodiment, the AI-based method further comprising generating, by the one or more hardware processors, one or more actionable reports based on the analyzed analyte data with the one or more analytes. The one or more actionable reports are one or more suggestions on at least one of: decisions on health of the one or more resources, creation of one or more vaccines, one or more therapeutics, and supporting of one or more research activities.

In yet another embodiment, the AI-based method further comprising dynamically adjusting, by the one or more hardware processors, the one or more pre-defined patterns associated with the one or more pre-stored analytes of the pre-stored analyte data in the multianalyte database, based on the analyte data obtained from the one or more samples of the one or more resources over a period of time, using an adaptive sampling process.

In yet another embodiment, the AI-based method further comprising integrating, by the one or more hardware processors, an AI-based system (e.g., an AI-based vertically integrated pathogenic active surveillance system) with one or more external surveillance systems to exchange data to determine the biological status information indicative of the response of the one or more resources to each of the one or more pathogens

Another aspect of the present disclosure provides an artificial intelligence (AI) based system for automatically determining biological status information of one or more resources based on one or more pathogens using multianalyte database. The AI-based system includes one or more hardware processors and a memory. The memory is coupled to the one or more hardware processors. The memory comprises a plurality of subsystems in form of programmable instructions executable by the one or more hardware processors. The plurality of subsystems comprises a data obtaining subsystem configured to obtain analyte data from one or more samples collected from the one or more resources. The one or more samples are collected from one or more resources comprising at least one of: environments, humans and animals. The analyte data are obtained from the one or more samples through at least one of: one or more sensors, one or more detectors, manual and automated data collectors, and laboratory analysis. The analyte data comprises information associated with one or more analytes obtained from the one or more resources via the one or more samples of the one or more resources.

The plurality of subsystems further comprises a data storage subsystem configured to store the analyte data associated with the one or more analytes, in the multianalyte database. The multianalyte database comprises pre-stored analyte data associated with the one or more pre-stored analytes in one or more pre-defined patterns. Each of the one or more pre-defined patterns is one or more combinations of the one or more pre-stored analytes indicative of the one or more pathogens, historical analyte data from known pathogen exposures, and one or more reference patterns for pathogen identification.

The plurality of subsystems further comprises a data analyzing subsystem configured to analyze the obtained analyte data comprising the one or more analytes, to determine the biological status information indicative of a response of the one or more resources to each of the one or more pathogens using the AI model. For analyzing the obtained analyte data, the data analyzing subsystem is configured to ingest the obtained analyte data comprising the one or more analytes, at the AI model. In an embodiment, the one or more analytes at the AI model. The one or more analytes comprise at least one of: nucleic acid data, deoxyribonucleic acid (DNA), ribonucleic acid (RNA), messenger RNA (mRNA), and microRNA (miRNA), protein concentration, protein three dimensional (3D) structures, protein-protein interactions, ligand receptor binding, protein-protein interactions, ligand receptor binding, carbohydrates, disease state, molecules, and metadata. The metadata comprise at least one of: one or more species, one or more locations where the one or more samples are collected from the one or more resources, and one or more dates on which the one or more samples are collected.

For analyzing the obtained analyte data, the data analyzing subsystem is further configured to retrieve the pre-stored analyte data from the multianalyte database. For analyzing the obtained analyte data, the data analyzing subsystem is further configured to compare the obtained analyte data with the pre-stored analyte data, to identify the one or more pathogens, using the AI model. For analyzing the obtained analyte data, the data analyzing subsystem is further configured to correlate one or more analyte patterns in the obtained analyte data, with known pathogen signatures, to determine pathogen-specific signatures within the obtained analyte data, using the AI model.

For analyzing the obtained analyte data, the data analyzing subsystem is further configured to extract receptor binding characteristics from the obtained analyte data, using the AI model, by: (a) identifying receptor binding proteins and concentrations of the receptor binding proteins, within the obtained analyte data; (b) analyzing three dimensional protein structures in the obtained analyte data for determining binding domain compatibility; (c) evaluating ligand receptor binding profiles present in the obtained analyte data; and (d) generating one or more scores for binding affinity potential based on molecular interaction data within the obtained analyte data.

For analyzing the obtained analyte data, the data analyzing subsystem is further configured to determine host cell adherence capability from the obtained analyte data, using the AI model, by: (a) detecting adhesion protein markers and concentrations in the obtained analyte data; (b) analyzing protein-protein interaction profiles within the obtained analyte data for cell surface binding; (c) identifying attachment mechanism indicators present in the obtained analyte data; and (d) quantifying potential host cell adherence using molecular signatures found in the obtained analyte data.

For analyzing the obtained analyte data, the data analyzing subsystem is further configured to identify one or more virulence genetic factors comprising at least one of: virulence gene sequences, toxin-encoding genetic elements, immune evasion markers, pathogenicity indicators, in the obtained analyte data, using the AI model.

For analyzing the obtained analyte data, the data analyzing subsystem is further configured to classify the one or more pathogens comprising potential zoonotic pathogens from the obtained analyte data, using the AI model, by: (a) comparing the pathogen-specific signatures within the obtained analyte data against known zoonotic pathogen patterns; (b) identifying species-jumping genetic markers within the obtained analyte data; (c) detecting cross-species transmission indicators present in the obtained analyte data; and (d) classifying the potential zoonotic pathogens based on one or more patterns associated with the potential zoonotic pathogens determined in the obtained analyte data.

For analyzing the obtained analyte data, the data analyzing subsystem is further configured to assign one or more pandemic threat scores to the potential zoonotic pathogens based on at least one of: the receptor binding characteristics, the host cell adherence capability, and the one or more virulence genetic factors, using the AI model. For analyzing the obtained analyte data, the data analyzing subsystem is further configured to determine the biological status information indicative of a response of the one or more resources to each of the one or more pathogens comprising the potential zoonotic pathogens, based on the one or more pandemic threat scores assigned to the potential zoonotic pathogens, using the AI model.

The plurality of subsystems further comprises an output subsystem configured to provide the biological status information indicative of a response of the one or more resources to each of the one or more pathogens comprising the potential zoonotic pathogens, as an output to a display device associated with one or more users.

Yet another aspect of the present disclosure provides a non-transitory computer-readable storage medium having instructions stored therein that, when executed by a hardware processor, causes the processor to perform method steps as described above.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF ACCOMPANYING DRAWINGS

The disclosure will be described and explained with additional specificity and detail with the accompanying figures in which:

FIG. 1 illustrates an exemplary block diagram representation of a network architecture implementing an artificial intelligence (AI) based system for automatically determining biological status information of one or more resources based on one or more pathogens using multianalyte database, in accordance with an embodiment of the present disclosure;

FIG. 2 illustrates an exemplary block diagram representation of the AI-based system, such as those shown in FIG. 1, capable of automatically determining the biological status information of the one or more resources based on the one or more pathogens using the multianalyte database, in accordance with an embodiment of the present disclosure;

FIG. 3 illustrates an exemplary flow diagram representation of a method for automatically determining the biological status information of the one or more resources based on the one or more pathogens using the multianalyte database, according to an example embodiment of the present disclosure;

FIG. 4 illustrates a flow chart depicting an AI-based method for automatically determining the biological status information of the one or more resources based on the one or more pathogens using the multianalyte database, according to an example embodiment of the present disclosure; and

FIG. 5 illustrates an exemplary block diagram representation of a hardware platform for an implementation of the disclosed AI-based system, according to an example embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that one or more devices or sub-systems or elements or structures or components preceded by “comprises... a” does not, without more constraints, preclude the existence of other devices, sub-systems, additional sub-modules. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

A computer system (standalone, client, or server computer system) configured by an application may constitute a “module” (or “subsystem”) that is configured and operated to perform certain operations. In one embodiment, the “module” or “subsystem” may be implemented mechanically or electronically, so a module includes dedicated circuitry or logic that is permanently configured (within a special-purpose processor) to perform certain operations. In another embodiment, a “module” or s “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired), or temporarily configured (programmed) to operate in a certain manner and/or to perform certain operations described herein.

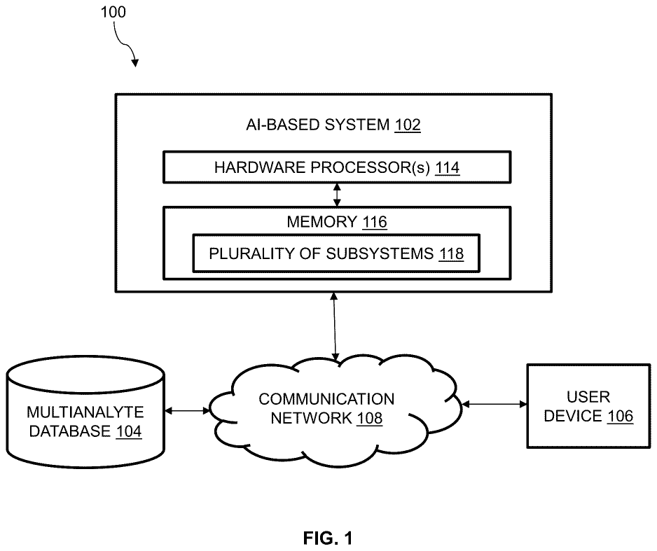

Referring now to the drawings, and more particularly to FIG. 1 through FIG. 5, where similar reference characters denote corresponding features consistently throughout the figures, there are shown preferred embodiments and these embodiments are described in the context of the following exemplary system and/or method.

Embodiments of the present disclosure provide an artificial intelligence (AI) based system and method for automatically determining biological status information of one or more resources based on one or more pathogens using multianalyte database. The present disclosure provides a comprehensive surveillance ecosystem comprising multiplexed miRNA assay, sample detecting devices, and the multianalyte database, which can be utilized to monitor the one or more pathogens. The present disclosure further utilizes other analytes including at least one of: nucleic acid data, deoxyribonucleic acid (DNA), ribonucleic acid (RNA), messenger RNA (mRNA), and microRNA (miRNA), protein concentration, protein three dimensional (3D) structures, protein-protein interactions, ligand receptor binding, carbohydrates, disease state, molecules, metadata, and the like, to monitor the one or more pathogens for determining the biological status information of the one or more resources.

FIG. 1 illustrates an exemplary block diagram representation of a network architecture 100 implementing the AI-based system 102 for automatically monitoring the one or more pathogens using the multianalyte database 104, in accordance with an embodiment of the present disclosure. The AI-based system 102 may be an AI-based vertically integrated pathogenic active surveillance system 102. According to FIG. 1, the network architecture 100 may include the AI-based system 102, the multianalyte database 104, and a user device 106. The AI-based system 102 may be communicatively coupled to the multianalyte database 104, and the user device 106 via a communication network 108. The communication network 108 may be a wired communication network and/or a wireless communication network. In another embodiment, the multianalyte database 104 may be a part of the AI-based system 102.

The multianalyte database 104 may include, but is not limited to, micro-ribonucleic acid (miRNA) concentrations, miRNA signature sequences, miRNA profiling data, a miRNA response, a miRNA absence, miRNA quantitative levels, miRNA concentrations, miRNA expression patterns, miRNA relationships within the miRNA profiling data, any other content (e.g., nucleic acid data, deoxyribonucleic acid (DNA), ribonucleic acid (RNA), messenger RNA (mRNA), and microRNA (miRNA), protein concentration, protein three dimensional (3D) structures, protein-protein interactions, ligand receptor binding, carbohydrates, disease state, molecules, and metadata), and combinations thereof. The multianalyte database 104 may be any kind of database such as, but are not limited to, relational databases, dynamic databases, monetized databases, scalable databases, cloud databases, distributed databases, any other databases, and combination thereof.

Further, the user device 106 may be associated with, but not limited to, a user, an individual, an administrator, a vendor, a technician, a health care worker, a caretaker, a patient, a supervisor, a team, an entity, a facility, and the like. The user device 106 may be used to provide input and/or receive output to/from the AI-based system 102, and/or to the multianalyte database 104. The user device 106 may present to the user one or more user interfaces for the user to interact with the AI-based system 102 and/or to the multianalyte database 104 for the analytes analyzing needs. In an embodiment, the user device 106 may include one or more sensors and one or more detectors, for capturing analyte data from one or more samples of one or more resources. The user device 106 may be at least one of, an electrical, an electronic, an electromechanical, and a computing device. The user device 106 may include, but is not limited to, a mobile device, a smartphone, a Personal Digital Assistant (PDA), a tablet computer, a phablet computer, a wearable computing device, a Virtual Reality/Augmented Reality (VR/AR) device, a laptop, a desktop, a server, a quantum computer, and the like. The entities and the facility may include, but are not limited to, a hospital, a healthcare facility, a laboratory facility, an e-commerce company, a merchant organization, an airline company, a hotel booking company, a company, an outlet, a manufacturing unit, an enterprise, an organization, an educational institution, a secured facility, a warehouse facility, a supply chain facility, any other facility and the like.

Further, the AI-based system 102 may be implemented by way of a single device or a combination of multiple devices that may be operatively connected or networked together. The AI-based system 102 may be implemented in hardware or a suitable combination of hardware and software.

The AI-based system 102 is initially configured to obtain the analyte data from the one or more samples collected from the one or more resources. In an embodiment, the one or more samples are collected from the one or more resources including at least one of: environments (e.g., air, soil, water, and the like), humans, and animals (e.g., livestock animals, poultry, pets. and the like). In an embodiment, the analyte data are obtained from the one or more samples through at least one of: the one or more sensors, the one or more detectors, manual and automated data collectors, and laboratory analysis. In an embodiment, the analyte data may include information associated with one or more analytes obtained from the one or more resources via the one or more samples of the one or more resources.

The AI-based system 102 is further configured to store the analyte data associated with the one or more analytes, in the multianalyte database 104. In an embodiment, the multianalyte database 104 includes pre-stored analyte data associated with the one or more pre-stored analytes in one or more pre-defined patterns. In an embodiment, each of the one or more pre-defined patterns is one or more combinations of the one or more pre-stored analytes, indicative of the one or more pathogens, historical analyte data from known pathogen exposures, and one or more reference patterns for pathogen identification.

The AI-based system 102 is further configured to analyze the obtained analyte data including the one or more analytes, to determine the biological status information indicative of a response of the one or more resources to each of the one or more pathogens using the AI model. For analyzing the obtained analyte data, the AI-based system 102 is configured to ingest the obtained analyte data comprising the one or more analytes, at the AI model. In an embodiment, the one or more analytes at the AI model. The one or more analytes comprise at least one of: nucleic acid data, deoxyribonucleic acid (DNA), ribonucleic acid (RNA), messenger RNA (mRNA), and microRNA (miRNA), protein concentration, protein three dimensional (3D) structures, protein-protein interactions, ligand receptor binding, protein-protein interactions, ligand receptor binding, carbohydrates, disease state, molecules, and metadata. The metadata may include at least one of: one or more species, one or more locations where the one or more samples are collected from the one or more resources, and one or more dates on which the one or more samples are collected.

For analyzing the obtained analyte data, the AI-based system 102 is further configured to retrieve the pre-stored analyte data from the multianalyte database 104. For analyzing the obtained analyte data, the AI-based system 102 is further configured to compare the obtained analyte data with the pre-stored analyte data, to identify the one or more pathogens, using the AI model. For analyzing the obtained analyte data, the AI-based system 102 is further configured to correlate one or more analyte patterns in the obtained analyte data, with known pathogen signatures, to determine pathogen-specific signatures within the obtained analyte data, using the AI model.

For analyzing the obtained analyte data, the AI-based system 102 is further configured to extract receptor binding characteristics from the obtained analyte data, using the AI model, by: (a) identifying receptor binding proteins and concentrations of the receptor binding proteins, within the obtained analyte data; (b) analyzing three dimensional protein structures in the obtained analyte data for determining binding domain compatibility; (c) evaluating ligand receptor binding profiles present in the obtained analyte data; and (d) generating one or more scores for binding affinity potential based on molecular interaction data within the obtained analyte data.

For analyzing the obtained analyte data, the AI-based system 102 is further configured to determine host cell adherence capability from the obtained analyte data, using the AI model, by: (a) detecting adhesion protein markers and concentrations in the obtained analyte data; (b) analyzing protein-protein interaction profiles within the obtained analyte data for cell surface binding; (c) identifying attachment mechanism indicators present in the obtained analyte data; and (d) quantifying potential host cell adherence using molecular signatures found in the obtained analyte data.

For analyzing the obtained analyte data, the AI-based system 102 is further configured to identify one or more virulence genetic factors comprising at least one of: virulence gene sequences, toxin-encoding genetic elements, immune evasion markers, pathogenicity indicators, in the obtained analyte data, using the AI model.

For analyzing the obtained analyte data, the AI-based system 102 is further configured to classify the one or more pathogens comprising potential zoonotic pathogens from the obtained analyte data, using the AI model, by: (a) comparing the pathogen-specific signatures within the obtained analyte data against known zoonotic pathogen patterns; (b) identifying species-jumping genetic markers within the obtained analyte data; (c) detecting cross-species transmission indicators present in the obtained analyte data; and (d) classifying the potential zoonotic pathogens based on one or more patterns associated with the potential zoonotic pathogens determined in the obtained analyte data.

For analyzing the obtained analyte data, the AI-based system 102 is further configured to assign one or more pandemic threat scores to the potential zoonotic pathogens based on at least one of: the receptor binding characteristics, the host cell adherence capability, and the one or more virulence genetic factors, using the AI model. For analyzing the obtained analyte data, the AI-based system 102 is further configured to determine the biological status information indicative of a response of the one or more resources to each of the one or more pathogens comprising the potential zoonotic pathogens, based on the one or more pandemic threat scores assigned to the potential zoonotic pathogens, using the AI model.

This new approach using the multianalyte profiles and pandemic threat scores may guide diagnostic assays that is used in the active surveillance system that prevents the next pandemic. Typically, the current approaches use statistical analysis of single analyte databases to identify markers for diagnostic development. An issue with single analyte analysis is its inability to identify complex diagnostic profiles as only one analyte is analyzed and there is no analysis to compute a pandemic risk score for emerging zoonotic pathogens.

The AI-based system 102 is further configured to provide the biological status information indicative of a response of the one or more resources to each of the one or more pathogens comprising the potential zoonotic pathogens, as an output to a display device associated with one or more users.

The AI-based system 102 is an active surveillance system, which shortens the time to response, using diagnostics for multianalyte profiles that are generated by an AI analysis of the multianalyte database. The multianalyte database may include the analyte data from multiple species (e.g., wildlife, livestock, pets, and humans), multiple analytes (e.g., miRNA, mRNA, DNA, proteins, 3-D protein structure, molecules, and metadata). The analyte data are analyzed by the AI model to identify profiles consisting of multiple analyte statuses to detect pathogen exposure and/or infection. In an embodiment, the multianalyte database is structured in a forward-looking architecture that allows implementation in binary computers and q-bits for compatibility with quantum computing. In another embodiment, as the multianalyte database 104 stores information associated with the humans, the information may be tagged with traceability and provenance markers that is used to assign fractional ownership for humans whose data was used to train the AI models.

The AI-based system 102 includes one or more hardware processor(s) 114 and a memory 116. The memory 116 may include a plurality of subsystems 118. The AI-based system 102 may be a hardware device including the hardware processor 114 executing machine-readable program instructions for monitoring the one or more pathogens. Execution of the machine-readable program instructions by the hardware processor 114 may enable the proposed AI-based system 102 to analyze the analyte data for determining the biological status information indicative of the response of the one or more resources to each of the one or more pathogens using the AI model. The “hardware” may comprise a combination of discrete components, an integrated circuit, an application-specific integrated circuit, a field-programmable gate array, a digital signal processor, or other suitable hardware. The software may comprise one or more objects, agents, threads, lines of code, subroutines, separate software applications, two or more lines of code, or other suitable software structures operating in one or more software applications or on one or more processors.

The hardware processor 114 may include, for example, microprocessors, microcomputers, microcontrollers, digital signal processors, central processing units, state machines, logic circuits, and/or any devices that manipulate data or signals based on operational instructions. Among other capabilities, the one or more hardware processor 114 may fetch and execute computer-readable instructions in a memory operationally coupled with the AI-based system 102 for performing tasks such as data processing, input/output processing, and/or any other functions. Any reference to a task in the present disclosure may refer to an operation being or that may be performed on data.

Though few components and subsystems are disclosed in FIG. 1, there may be additional components and subsystems which is not shown, such as, but not limited to, assets, machinery, instruments, facility equipment, life safety devices, intensive care devices, treatment devices, emergency management devices, health care devices, laboratory devices, testing kits, any other devices, and combination thereof. The person skilled in the art should not be limiting the components/subsystems shown in FIG. 1. Although FIG. 1 illustrates the AI-based system 102, and the user device 106 connected to the multianalyte database 104, one skilled in the art can envision that the AI-based system 102, and the user device 106 can be connected to several user devices located at different locations and several multianalyte databases via the communication network 108.

Those of ordinary skilled in the art will appreciate that the hardware depicted in FIG. 1 may vary for particular implementations. For example, other peripheral devices such as an optical disk drive and the like, local area network (LAN), wide area network (WAN), wireless (e.g., wireless-fidelity (Wi-Fi)) adapter, graphics adapter, disk controller, input/output (I/O) adapter also may be used in addition or place of the hardware depicted. The depicted example is provided for explanation only and is not meant to imply architectural limitations concerning the present disclosure.

Those skilled in the art will recognize that, for simplicity and clarity, the full structure and operation of all data processing systems suitable for use with the present disclosure are not being depicted or described herein. Instead, only so much of the AI-based system 102 as is unique to the present disclosure or necessary for an understanding of the present disclosure is depicted and described. The remainder of the construction and operation of the AI-based system 102 may conform to any of the various current implementations and practices that were known in the art.

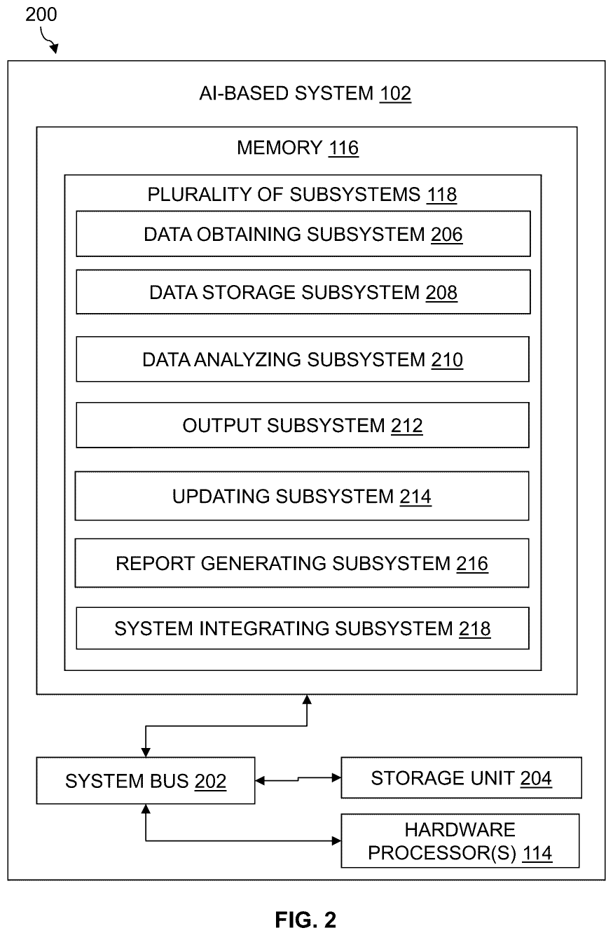

FIG. 2 illustrates an exemplary block diagram 200 representation of the AI-based system 102, such as those shown in FIG. 1, capable of automatically determining the biological status information of the one or more resources based on the one or more pathogens using the multianalyte database 104, in accordance with an embodiment of the present disclosure. The AI-based system 102 may also function as a computer-implemented system (hereinafter referred to as the system 102). The AI-based system 102 comprises the one or more hardware processors 114, the memory 116, and a storage unit 204. The one or more hardware processors 114, the memory 116, and the storage unit 204 are communicatively coupled through a system bus 202 or any similar mechanism. The memory 116 comprises a plurality of subsystems 118 in the form of programmable instructions executable by the one or more hardware processors 114.

Further, the plurality of subsystems 118 includes a data obtaining subsystem 206, a data storage subsystem 208, a data analyzing subsystem 210, an output subsystem 212,, an updating subsystem 214, a report generating subsystem 216, and a system integrating subsystem 218.

In an embodiment, the AI-based system 102 includes the plurality of subsystems 118 that communicate mutually among each other in real-time and uses the artificial intelligence model to monitor the one or more pathogens for determining the biological status information of the one or more resources.

The one or more hardware processors 114, as used herein, means any type of computational circuit, such as, but not limited to, a microprocessor unit, microcontroller, complex instruction set computing microprocessor unit, reduced instruction set computing microprocessor unit, very long instruction word microprocessor unit, explicitly parallel instruction computing microprocessor unit, graphics processing unit, digital signal processing unit, or any other type of processing circuit. The one or more hardware processors 114 may also include embedded controllers, such as generic or programmable logic devices or arrays, application-specific integrated circuits, single-chip computers, and the like.

The memory 116 may be a non-transitory volatile memory and a non-volatile memory. The memory 116 may be coupled to communicate with the one or more hardware processors 114, such as being a computer-readable storage medium. The one or more hardware processors 114 may execute machine-readable instructions and/or source code stored in the memory 116. A variety of machine-readable instructions may be stored in and accessed from the memory 116. The memory 116 may include any suitable elements for storing data and machine-readable instructions, such as read-only memory, random access memory, erasable programmable read-only memory, electrically erasable programmable read-only memory, a hard drive, a removable media drive for handling compact disks, digital video disks, diskettes, magnetic tape cartridges, memory cards, and the like. In the present embodiment, the memory 116 includes the plurality of subsystems 118 stored in the form of machine-readable instructions on any of the above-mentioned storage media and may be in communication with and executed by the one or more hardware processors 114.

The storage unit 204 may be a cloud storage or a database such as those shown in FIG. 1. The storage unit 204 may store the one or more analytes including at least one of: micro-ribonucleic acid (miRNA) concentrations, miRNA signature sequences, miRNA profiling data, a miRNA response, a miRNA absence, miRNA quantitative levels, miRNA concentrations, miRNA expression patterns, miRNA relationships within the miRNA profiling data, any other content (e.g., nucleic acid data, deoxyribonucleic acid (DNA), ribonucleic acid (RNA), messenger RNA (mRNA), and microRNA (miRNA), protein concentration, protein three dimensional (3D) structures, protein-protein interactions, ligand receptor binding, carbohydrates, disease state, molecules, and metadata), and combinations thereof. The storage unit 204 may be any kind of database such as, but are not limited to, relational databases, dynamic databases, monetized databases, scalable databases, cloud databases, distributed databases, any other databases, and combination thereof.

In an exemplary embodiment, the plurality of subsystems 118 includes the data obtaining subsystem 206 that is communicatively connected to the one or more hardware processors 114. The data obtaining subsystem 206 is configured to obtain the analyte data from the one or more samples collected from the one or more resources. The analyte data is the comprehensive molecular and biochemical information extracted from biological, environmental, or clinical samples that contains measurable quantities and characteristics of specific target molecules or substances being analyzed. This analyte data encompasses detailed measurements, concentrations, structural information, and molecular profiles of various biological components including nucleic acids such as deoxyribonucleic acid (DNA), ribonucleic acid (RNA), messenger RNA (mRNA), and microRNA (miRNA), protein concentrations and three-dimensional protein structures, protein-protein interactions and ligand receptor binding profiles, carbohydrates and metabolites, disease state indicators, and associated metadata such as species identification, sample collection locations, and collection dates. The analyte data represents the fundamental informational output generated when biological samples undergo analysis through sensors, detectors, automated data collection systems, and laboratory analysis procedures, providing the raw material for subsequent artificial intelligence processing and pathogen identification. For example, when a blood sample from a COVID-19 patient is processed, the resulting analyte data includes specific SARS-CoV-2 viral RNA sequence information, elevated interleukin-6 protein concentration measurements, decreased lymphocyte count data, specific antibody binding profiles, inflammatory cytokine levels, and temporal metadata indicating when and where the sample was collected, collectively forming a comprehensive molecular dataset that describes the biological state and pathogen response characteristics of that particular sample.

In an embodiment, the one or more samples are collected from the one or more resources including at least one of: the environments (e.g., air, soil, water, and the like), the humans, and the animals (e.g., livestock animals, poultry, and the like). In an embodiment, the analyte data are obtained from the one or more samples through at least one of: the one or more sensors, the one or more detectors, manual and automated data collectors, and laboratory analysis. In an embodiment, the analyte data may include the information associated with the one or more analytes obtained from the one or more resources via the one or more samples of the one or more resources. In an embodiment, the analyte data is obtained from one or more resources over a period of time. In an embodiment, the analyte data are received from at least two or more analytes.

In an embodiment, the one or more resources may be dissimilar to one another. In an embodiment, the analyte data used to establish the multianalyte database 104, are obtained over a plurality of time from the one or more analytes so that the multianalyte database 104 may be comprised of the one or more analytes from the one or more resources. In an embodiment, the one or more analytes may include at least one of: nucleic acid data, deoxyribonucleic acid (DNA), ribonucleic acid (RNA), messenger RNA (mRNA), and microRNA (miRNA), protein concentration, protein three dimensional (3D) structures, protein-protein interactions, ligand receptor binding, carbohydrates, disease state, molecules, and metadata. In an embodiment, the metadata may include at least one of: one or more species, one or more locations where the one or more samples are collected from the one or more resources, and one or more dates on which the one or more samples are collected.

The data obtaining subsystem 206 is a specialized computational and hardware component within the AI-based system 102 that functions as the primary data acquisition interface, configured to obtain the analyte data from the one or more samples collected from the one or more resources, where these one or more resources represent diverse biological and environmental sources comprising at least one of: environments, humans and animals, such as contaminated soil from agricultural fields, blood specimens from hospital patients, or tissue samples from wildlife populations. This data obtaining subsystem 206 operates through multiple data extraction pathways where the analyte data are obtained from the one or more samples through at least one of: one or more sensors (such as biosensors that detect molecular concentrations in real-time, environmental air quality sensors, water contamination sensors, temperature and humidity sensors, pH sensors, and the like), one or more detectors (such as mass spectrometry detectors that identify protein structures, flow cytometers, X-ray detectors, gamma ray detectors, Infrared detectors, and the like), manual and automated data collectors (such as automated robotic systems that process thousands of samples simultaneously, automated liquid handling systems, high-throughput screening platforms, AUTOMZATED ELISA processors, and the like), and laboratory analysis (such as trained laboratory technicians who manually extract nucleic acids using standardized protocols, DNA sequencing, RNA analysis, protein quantification, and the like). The resulting output consists of comprehensive molecular information where the analyte data comprises information associated with one or more analytes obtained from the one or more resources via the one or more samples of the one or more resources. For example, when a saliva sample collected from an infected human patient (the resource) yields miRNA expression profiles, protein concentration measurements, DNA sequences, and metabolite data (the analytes) that collectively represent the molecular information extracted from that specific human resource through the collected saliva sample, thereby providing the foundational dataset required for subsequent AI-driven pathogen identification and biological status determination.

In an exemplary embodiment, the plurality of subsystems 118 further includes the data storage subsystem 208 that is communicatively connected to the one or more hardware processors 114. The data storage subsystem 208 is configured to store the analyte data associated with the one or more analytes, in the multianalyte database 104. In an embodiment, the multianalyte database 104 may include the pre-stored analyte data associated with the one or more pre-stored analytes in the one or more pre-defined patterns. In an embodiment, each of the one or more pre-defined patterns is one or more combinations of the one or more pre-stored

analytes indicative of the one or more pathogens, historical analyte data from known pathogen exposures, and one or more reference patterns for pathogen identification. The one or more pathogens refers to disease-causing microorganisms or infectious agents that have the capability to cause illness, infection, or disease in host organisms, including viruses (such as SARS-CoV-2, influenza viruses, HIV), bacteria (such as Escherichia coli, Salmonella, Staphylococcus aureus), fungi (such as Candida albicans, Aspergillus species), parasites (such as Plasmodium malaria, Toxoplasma gondii), prions (misfolded proteins causing diseases like BSE), and other pathogenic microorganisms that can infect humans, animals, or plants, cause cellular damage, disrupt normal biological functions, and potentially lead to morbidity or mortality in affected hosts.

In an embodiment, the analyte data stored in the multianalyte database 104 may include at least one of: raw data, unprocessed data, and processed data. The analyte data stored in the multianalyte database 104 may be raw and unprocessed analyte data that may be analyzed to determine information associated with at least one of: presence, concentration, and pattern of the one or more analytes, to provide the new information. In an embodiment, the analyte data stored in the multianalyte database 104 may include at least one analyte or more than one analyte.

The data storage subsystem 208 is a specialized database management component within the AI-based system 102 that functions as the central repository for organizing, preserving, and managing molecular information. The data storage subsystem 208 is configured to store the analyte data associated with the one or more analytes, in the multianalyte database 104 where the data storage subsystem 208 systematically organizes and preserves molecular information related to specific biological molecules such as DNA sequences, protein concentrations, miRNA profiles, and metabolite data within a comprehensive database structure designed to handle diverse biological data types simultaneously. The multianalyte database 104 is a comprehensive data repository that organizes and maintains molecular information from multiple types of biological markers in structured formats, enabling efficient data retrieval, pattern recognition, and analytical processing for pathogen surveillance and biological status assessment. The multianalyte database 104 comprises pre-stored analyte data associated with the one or more pre-stored analytes in one or more pre-defined patterns meaning the multianalyte database 104 contains previously collected, processed, and validated molecular information from historical samples and research studies that have been organized according to specific, previously identified molecular signatures or characteristic combinations of biological molecules, such as particular miRNA expression levels combined with specific protein concentrations and genetic markers that form recognizable molecular fingerprints. Furthermore, each of the one or more pre-defined patterns is one or more combinations of the one or more pre-stored analytes indicative of the one or more pathogens signifying that every catalogued molecular signature represents specific arrangements or groupings of biological molecules like elevated cytokine proteins combined with decreased immune cell markers and specific viral RNA sequences that collectively indicate the presence of particular disease-causing organisms such as SARS-CoV-2, influenza virus, or bacterial pathogens like E. coli.

Additionally, the multianalyte database 104 includes historical analyte data from known pathogen exposures which consists of previously documented and verified molecular information from confirmed cases of pathogen infections, outbreaks, and exposures, such as the specific miRNA expression profiles, protein biomarkers, and genetic sequences that were observed and recorded in patients who were definitively diagnosed with Ebola, anthrax, or pandemic influenza during past disease events. Finally, the data storage subsystem 208 incorporates one or more reference patterns for pathogen identification representing standardized, validated molecular signatures that serve as definitive benchmarks and comparison templates for recognizing and identifying specific pathogens, such as the characteristic combination of genetic sequences, protein markers, and metabolic indicators that definitively distinguish and identify particular viruses like Zika or bacteria like tuberculosis in biological samples.

For example, the data storage subsystem 208 configured to store the analyte data associated with the one or more analytes, in the multianalyte database 104, operates within a global pandemic surveillance network where the data storage subsystem 208 systematically organizes and preserves incoming molecular information from COVID-19 outbreak investigations, storing analyte data such as SARS-CoV-2 viral RNA sequences, spike protein concentrations, inflammatory cytokine levels, and patient antibody titers in structured database tables with standardized formats and cross-referencing capabilities. The multianalyte database 104 comprises the pre-stored analyte data associated with the one or more pre-stored analytes in one or more pre-defined patterns containing previously catalogued molecular signatures such as Pattern Alpha which combines elevated IL-6 protein levels above 50 pg/mL with decreased lymphocyte counts below 1000 cells/μL and specific viral RNA sequences indicating severe COVID-19 infection, Pattern Beta which integrates increased D-dimer levels above 500 ng/mL with elevated ferritin concentrations and particular miRNA expression profiles indicating cytokine storm syndrome, and Pattern Gamma which correlates specific antibody responses with viral load measurements and inflammatory markers indicating vaccine breakthrough infections. Furthermore, each of the one or more pre-defined patterns is one or more combinations of the one or more pre-stored analytes, indicative of the one or more pathogens where Pattern Alpha's combination of three specific analytes collectively indicates SARS-CoV-2 Delta variant infection, Pattern Beta's four-analyte combination indicates severe inflammatory response to coronavirus, and Pattern Gamma's five-analyte combination indicates Omicron variant breakthrough infection in vaccinated individuals. The multianalyte database 104 also incorporates historical analyte data from known pathogen exposures including molecular profiles from the original Wuhan COVID-19 outbreak showing characteristic miRNA expression patterns and protein biomarkers from confirmed patient samples, analyte data from the 2021 Delta variant surge documenting specific genetic mutations and immune response markers, and molecular signatures from Omicron infections during 2022 showing altered viral RNA levels and modified antibody responses. Additionally, the data storage subsystem 208 maintains one or more reference patterns for pathogen identification such as the definitive molecular signature for identifying SARS-CoV-2 variants consisting of spike protein genetic sequences combined with nucleocapsid protein markers and specific host immune response indicators, the standard identification profile for distinguishing COVID-19 from influenza including differential cytokine responses and viral RNA characteristics, and the benchmark pattern for detecting vaccine-resistant variants combining mutation-specific genetic sequences with altered antibody binding profiles and modified host cell response indicators.

In an exemplary embodiment, the plurality of subsystems 118 further includes the data analyzing subsystem 210 that is communicatively connected to the one or more hardware processors 114. The data analyzing subsystem 210 is configured to analyze the obtained analyte data including the one or more analytes, to determine the biological status information indicative of the response of the one or more resources to each of the one or more pathogens using the AI model. In an embodiment, analyzing the obtained analyte data may include determination of at least one of: a presence of at least one analyte and a concentration of the at least one analyte.

In an embodiment, the raw/unprocessed data may be analyzed to generate the analyzed analyte data and store the analyzed analyte data in the multianalyte database 104. In another embodiment, the raw/unprocessed data may be analyzed to determine the presence of at least one analyte using the AI model, In yet another embodiment, the raw/unprocessed data may be analyzed to determine the presence of at least one source using the AI model. In yet another embodiment, the raw/unprocessed data may be analyzed to determine the presence of at least two analytes using the AI model. In yet another embodiment, the raw/unprocessed data may be analyzed to determine the presence of at least two similar sources using the AI model. In yet another embodiment, the raw/unprocessed data may be analyzed to determine the presence of at least two dissimilar sources using the AI model.

The data analyzing subsystem 210 is a specialized computational component within the AI-based system 102 that serves as the primary analytical engine, specifically designed to systematically process and examine the molecular information contained within the obtained analyte data such as DNA sequences, protein concentrations, miRNA expression levels, and metabolite profiles through advanced artificial intelligence algorithms to generate comprehensive health assessments that describe how different source entities including humans, animals, and environmental systems react when exposed to specific disease-causing organisms.

This data analyzing subsystem 210 operates through multiple sophisticated AI model processes including Deep Learning Convolutional Neural Networks that systematically analyze the obtained analyte data by applying multiple layers of computational filters to detect complex molecular patterns within nucleic acid sequences and protein structures, where the first convolutional layer identifies basic sequence motifs and protein domains, the second layer combines these features to recognize larger molecular complexes and interaction networks, and the final layers integrate all molecular information to classify pathogen types and predict host response severity through backpropagation learning algorithms that continuously refine pattern recognition accuracy based on training data from thousands of confirmed pathogen cases.

The data analyzing subsystem 210 also employs Multi-Task Learning Neural Networks that simultaneously process multiple aspects of the obtained analyte data by sharing computational resources across related analytical tasks, where shared hidden layers learn common molecular representations from protein concentrations and genetic sequences, while task-specific output layers generate distinct predictions for pathogen identification, infection severity assessment, and treatment response forecasting, enabling the data analyzing subsystem 210 to leverage correlations between different analytical objectives to improve overall accuracy in determining biological status information for each monitored resource.

Additionally, the data analyzing subsystem 210 utilizes Ensemble Random Forest Models that analyze the obtained analyte data through hundreds of decision trees operating in parallel, where each tree processes different subsets of molecular features such as specific protein markers, genetic variants, and metabolite concentrations to make independent predictions about pathogen presence and host response, and the final biological status determination results from aggregating votes across all trees using weighted averaging algorithms that account for each tree's historical accuracy on similar molecular patterns, thereby providing robust and reliable biological status information that accounts for uncertainty and variability in the obtained analyte data.

Furthermore, the data analyzing subsystem 210 incorporates Recurrent Neural Networks with Long Short-Term Memory that process temporal sequences within the obtained analyte data by maintaining memory states that track how molecular concentrations and genetic expressions change over time, where input gates selectively incorporate new analyte measurements, forget gates discard outdated molecular information, and output gates generate updated biological status predictions that account for disease progression patterns and treatment response trajectories observed in the monitored resources.

For example, when processing blood samples from a hospital during a COVID-19 outbreak, this data analyzing subsystem 210 analyzes the obtained analyte data containing elevated cytokine protein levels, specific SARS-CoV-2 viral RNA sequences, antibody titers, and inflammatory biomarkers through these four AI models working in coordination to determine the biological status information showing that Patient A exhibits severe immune response with cytokine storm patterns requiring immediate intensive care intervention, Patient B demonstrates moderate infection with controlled inflammatory response and early antibody production suitable for monitored outpatient treatment, and Patient C displays recovery-phase characteristics with declining viral RNA loads and robust neutralizing antibody levels indicating successful immune clearance, thereby providing comprehensive biological status information indicative of how each human resource is responding to the SARS-CoV-2 pathogen through systematic multi-model AI analysis.

For analyzing the obtained analyte data to determine the biological status information indicative of the response of the one or more resources to each of the one or more pathogens, using the AI model, the data analyzing subsystem 210 is configured to ingest the obtained analyte data including the one or more analytes at the AI model. In an embodiment, the one or more analytes comprise at least one of: nucleic acid data, deoxyribonucleic acid (DNA), ribonucleic acid (RNA), messenger RNA (mRNA), and microRNA (miRNA), protein concentration, protein three dimensional (3D) structures, protein-protein interactions, ligand receptor binding, protein-protein interactions, ligand receptor binding, carbohydrates, disease state, molecules, and metadata. The metadata may include at least one of: one or more species, one or more locations where the one or more samples are collected from the one or more resources, and one or more dates on which the one or more samples are collected.

The process of ingesting the obtained analyte data comprising the one or more analytes at the AI model represents the systematic computational intake and preprocessing phase where specialized hardware processors feed the comprehensive molecular information extracted from biological samples directly into the AI-based model for analysis, involving data formatting, normalization, and preparation steps that ensure the diverse molecular measurements can be properly processed by machine learning algorithms. The AI-model for analysis operates through a sophisticated multi-stage computational pipeline that transforms raw molecular measurements into standardized, machine-readable formats suitable for advanced algorithmic processing. The data formatting stage involves converting diverse molecular information from various laboratory instruments and detection systems into unified data structures, where nucleic acid sequences from DNA sequencers are transformed into standardized FASTA or FASTQ formats, protein concentration measurements from mass spectrometers are converted into numerical arrays with consistent units (ng/mL, μg/mL), three-dimensional protein structures from crystallography are encoded into PDB coordinate files, and metadata information is structured into standardized taxonomic codes, geographic coordinates, and ISO date formats that enable consistent cross-platform analysis.

The normalization process systematically adjusts for technical variations and measurement scales across different analyte types by applying statistical transformations such as z-score normalization to protein concentration data to account for instrument sensitivity differences, log-transformation to miRNA expression levels to handle exponential concentration ranges, batch effect correction algorithms to eliminate systematic biases between different collection dates and locations, and feature scaling techniques that ensure DNA sequence similarity scores, protein binding affinities, and carbohydrate concentrations are weighted appropriately despite having vastly different numerical ranges and measurement units. The preparation steps involve comprehensive data quality assessment and feature engineering processes where missing values in analyte measurements are imputed using advanced algorithms, outlier detection methods identify and handle anomalous readings that could skew AI model performance, dimensionality reduction techniques such as principal component analysis compress high-dimensional protein interaction networks and metabolic pathway data into manageable feature sets, and temporal alignment algorithms synchronize time-series analyte measurements from samples collected at different dates to enable meaningful longitudinal analysis. These integrated preprocessing operations ensure that when the AI model receives the ingested analyte data, the machine learning algorithms can effectively process the standardized molecular information to identify pathogen signatures, classify infection types, and predict biological responses without being hindered by technical artifacts, measurement inconsistencies, or format incompatibilities that would otherwise compromise the accuracy of pathogen detection and biological status determination across diverse sample types and collection scenarios.

The one or more analytes comprise at least one of: nucleic acid data, deoxyribonucleic acid (DNA), ribonucleic acid (RNA), messenger RNA (mRNA), and microRNA (miRNA), protein concentration, protein three dimensional (3D) structures, protein-protein interactions, ligand receptor binding, protein-protein interactions, ligand receptor binding, carbohydrates, disease state, molecules, and metadata. The nucleic acid data refers to comprehensive information about nucleic acid molecules including sequence data, structural information, concentration measurements, and functional characteristics of DNA and RNA molecules found in biological samples. The Deoxyribonucleic acid (DNA) is the double-stranded nucleic acid molecule that contains the genetic instructions for the development, functioning, and reproduction of all known living organisms, consisting of four nucleotide bases (adenine, thymine, guanine, cytosine) arranged in specific sequences. The Ribonucleic acid (RNA) is a single-stranded nucleic acid molecule that plays crucial roles in gene expression, protein synthesis, and cellular regulation, consisting of four nucleotide bases (adenine, uracil, guanine, cytosine) and existing in various forms including messenger RNA, transfer RNA, and ribosomal RNA. The Messenger RNA (mRNA) is a specific type of RNA molecule that carries genetic information from DNA to ribosomes for protein synthesis, serving as the template that determines the amino acid sequence of proteins during translation.

The MicroRNA (miRNA) are small, non-coding RNA molecules approximately 20-24 nucleotides in length that regulate gene expression by binding to complementary sequences on target mRNA molecules, thereby controlling protein production and cellular processes. The protein concentration refers to the quantitative measurement of protein amounts present in biological samples, typically expressed in units such as mg/mL, μg/mL, or ng/mL, indicating the abundance of specific proteins or total protein content. The protein three dimensional (3D) structures are the spatial arrangements and conformations of protein molecules, including primary sequence, secondary structures (alpha helices, beta sheets), tertiary folding patterns, and quaternary assemblies that determine protein function and interactions.

The protein-protein interactions are physical contacts and functional associations between two or more protein molecules that enable cellular processes, signaling pathways, and biological functions through specific binding, enzymatic reactions, or structural complexes. The ligand receptor binding refers to the specific molecular interactions between ligands (small molecules, hormones, neurotransmitters) and their corresponding receptor proteins, involving binding affinity, specificity, and conformational changes that trigger cellular responses. The Carbohydrates are organic compounds consisting of carbon, hydrogen, and oxygen atoms that serve as energy sources, structural components, and signaling molecules, including simple sugars, complex polysaccharides, and glycoproteins found in biological systems. The disease state refers to the pathological condition or health status of organisms, including the presence, severity, and progression of diseases, infections, or abnormal physiological conditions as determined through molecular markers and clinical indicators. The molecules are discrete chemical entities composed of atoms bonded together, including all types of biological and chemical compounds present in samples such as metabolites, lipids, hormones, toxins, and other molecular species relevant to biological analysis. The metadata are descriptive information and contextual data that provide additional details about the samples and analytical results, including sample collection parameters, experimental conditions, temporal information, and other relevant contextual factors that aid in data interpretation and analysis.

The one or more species refers to the taxonomic classification and biological identification of the organisms from which samples were collected, including specific species names, genus classifications, and biological categories such as Homo sapiens (humans), Gallus gallus domesticus (domestic chickens), Chiroptera species (bats), Escherichia coli (bacteria), SARS-CoV-2 (virus), or other taxonomic identifiers that specify the biological source of the analyte data. The one or more locations are the geographic coordinates, addresses, or spatial identifiers that specify where the sample collection activities took place, including GPS coordinates, city names, country designations, facility names, environmental sites, laboratory locations, hospital addresses, farm locations, wildlife habitats, or other geographic references that document the spatial origin of the collected samples. The one or more dates are temporal identifiers that specify when sample collection activities occurred, including specific calendar dates, timestamps, collection periods, or temporal ranges that document the timing of sample acquisition, such as “March 15, 2024,” “2024-03-15 14:30:00,” “Week 12, 2024,” or “March 10-17, 2024” that provide chronological context for the analytical data.

For example, when processing samples from a multi-species influenza outbreak investigation, the AI model ingests obtained analyte data containing H5N1 viral RNA sequences and protein concentrations from infected chickens, human antibody responses and cytokine levels from exposed farm workers, and environmental viral DNA from contaminated water sources, along with metadata indicating the samples originated from poultry farms in Iowa and Minnesota, were collected from chickens, humans, and water resources respectively, and were obtained during specific dates in March 2024, enabling the data analyzing subsystem 210 to analyze the comprehensive molecular and contextual information to determine cross-species transmission patterns and biological responses across different host species and environmental conditions.

For analyzing the obtained analyte data to determine the biological status information indicative of the response of the one or more resources to each of the one or more pathogens, using the AI model, the data analyzing subsystem 210 is further configured to retrieve the pre-stored analyte data from the multianalyte database 104. The process of retrieving the pre-stored analyte data from the multianalyte database 104 represents the systematic computational operation where specialized processing units access and extract previously stored molecular information from the comprehensive database repository, involving complex database query operations, indexing systems, and data retrieval algorithms that locate and fetch relevant historical molecular signatures, pathogen-specific patterns, and reference analyte profiles that match or relate to the current analysis requirements. This retrieval process operates through sophisticated database management systems that utilize structured query language commands, hash-based indexing for rapid molecular sequence lookup, relational database joins that connect analyte measurements with pathogen identifications, and distributed storage architectures that can simultaneously access multiple data partitions containing different types of molecular information such as nucleic acid libraries, protein concentration databases, and metadata repositories. The data analyzing subsystem 210 execute parallel retrieval operations that search through vast collections of pre-stored analyte data including historical pathogen outbreak records, validated molecular signatures from confirmed disease cases, reference patterns for known pathogens, and baseline analyte profiles from healthy control samples, using optimized search algorithms that can quickly identify relevant molecular patterns based on sequence similarity, concentration ranges, structural homology, and metadata matching criteria. The retrieved pre-stored analyte data encompasses comprehensive molecular information that has been previously validated and catalogued, including confirmed pathogen-specific genetic sequences, established protein biomarker concentrations, documented immune response patterns, verified metabolic signatures, and associated contextual information that provides the comparative foundation for analyzing new samples and identifying potential pathogen matches or novel biological responses.

For example, when analyzing blood samples from patients presenting with respiratory symptoms during flu season, the data analyzing subsystem 210 retrieve pre-stored analyte data from the multianalyte database containing thousands of previously confirmed influenza cases with their associated viral RNA sequences, cytokine concentration profiles, antibody response patterns, and inflammatory biomarker levels, along with reference data from seasonal H1N1 and H3N2 strains, pandemic H1N1 records from 2009, and baseline immune response profiles from healthy individuals, enabling the AI model to compare the current patient samples against this comprehensive repository of validated molecular information to accurately identify the specific influenza strain, assess infection severity, and predict patient response based on historical patterns observed in similar cases with matching analyte profiles.

For analyzing the obtained analyte data to determine the biological status information indicative of the response of the one or more resources to each of the one or more pathogens, using the AI model, the data analyzing subsystem 210 is further configured to compare the obtained analyte data with the pre-stored analyte data, to identify the one or more pathogens, using the AI model. The process of comparing the obtained analyte data with the pre-stored analyte data, to identify the one or more pathogens, using the AI model, represents the core analytical operation where the artificial intelligence model systematically evaluates and contrasts the molecular information extracted from current biological samples against the comprehensive repository of previously validated pathogen-specific molecular signatures stored in the multianalyte database 104, utilizing advanced machine learning algorithms to detect correlations, homologies, and characteristic molecular fingerprints that indicate the presence of specific disease-causing organisms. This comparison process operates by taking the obtained analyte data containing fresh molecular measurements such as viral RNA sequences, protein concentrations, and biomarker profiles from newly collected samples and systematically analyzing it against the pre-stored analyte data consisting of historical molecular signatures from confirmed pathogen cases. The AI model employs sophisticated algorithms including convolutional neural networks for sequence analysis, support vector machines for pattern classification, and ensemble learning methods that integrate multiple molecular features to generate comprehensive pathogen identification scores based on similarity measures, statistical correlations, and learned patterns from extensive training datasets. The AI model performs multi-dimensional comparisons by calculating sequence alignment scores between current viral RNA and archived pathogen genomes, computing correlation coefficients between fresh protein concentration profiles and established biomarker patterns, evaluating structural similarity indices between newly detected molecular features and known pathogen-associated signatures, and generating probability distributions that quantify the likelihood of specific pathogen presence based on the degree of molecular similarity and pattern matching accuracy. The identification process enables the system to distinguish between different pathogen types, infection stages, and variants by leveraging the AI model's ability to recognize complex molecular patterns and relationships that may not be apparent through traditional analytical methods.

The AI model employs convolutional neural networks for sequence analysis through a multi-layered computational architecture where the first convolutional layer applies sliding window filters across nucleic acid sequences to detect short sequence motifs and genetic patterns, the second layer combines these basic motifs to identify larger functional domains and regulatory elements, and deeper layers integrate complex sequence features to recognize complete viral genomes, bacterial signatures, or genetic variants, using pooling operations to reduce computational complexity while preserving critical sequence information and backpropagation algorithms to continuously refine the network's ability to distinguish between pathogen-specific genetic markers and benign sequence variations. The data analyzing subsystem 210 utilizes support vector machines for pattern classification by constructing high-dimensional decision boundaries that optimally separate different pathogen classes based on protein concentration profiles, biomarker combinations, and molecular feature vectors, where the algorithm maps analyte data points into higher-dimensional feature spaces using kernel functions such as radial basis functions or polynomial kernels, identifies the optimal hyperplane that maximizes the margin between different pathogen categories, and applies regularization techniques to prevent overfitting while ensuring robust classification performance across diverse sample types and pathogen variants. Additionally, the AI model implements ensemble learning methods that combine predictions from multiple individual algorithms including random forest models that aggregate decisions from hundreds of decision trees trained on different subsets of molecular features, gradient boosting machines that sequentially improve prediction accuracy by learning from previous model errors, and voting classifiers that integrate outputs from neural networks, support vector machines, and tree-based models using weighted averaging schemes based on each algorithm's historical performance on similar pathogen identification tasks. These sophisticated algorithms work synergistically where convolutional neural networks extract complex sequence patterns from DNA and RNA data, support vector machines classify protein and biomarker profiles into distinct pathogen categories, and ensemble methods integrate all molecular evidence to generate robust pathogen identification decisions with associated confidence scores and uncertainty estimates.

For example, when analyzing samples suspected of containing influenza virus, the convolutional neural network scans the obtained analyte data for characteristic hemagglutinin and neuraminidase gene sequences by applying learned filters that recognize H1N1, H3N2, and influenza B genetic signatures, the support vector machine classifies the protein concentration patterns including elevated interferon levels and specific antibody responses into influenza-positive or influenza-negative categories based on optimal decision boundaries learned from thousands of confirmed cases, and the ensemble learning method combines these sequence-based and protein-based predictions with additional evidence from metabolic markers and inflammatory biomarkers to generate a final identification decision showing 94% confidence for H3N2 influenza strain based on the integrated analysis of all molecular evidence from the obtained analyte data compared against the pre-stored analyte data repository.

In an embodiment, comparing the obtained analyte data with the pre-stored analyte data, to identify the one or more pathogens, using the AI model, the data analyzing subsystem 210 is further configured to match one or more analyte patterns in the obtained analyte data against the one or more pre-defined patterns, associated with the one or more pathogens, in the pre-stored analyte data. The process of matching one or more analyte patterns in the obtained analyte data against the one or more pre-defined patterns, associated with the one or more pathogens, in the pre-stored analyte data represents a sophisticated pattern recognition operation where the AI system systematically identifies characteristic molecular signature combinations from current samples and compares them with established pathogen-specific molecular templates stored in the database, utilizing multiple advanced artificial intelligence models to detect correlations and similarities between fresh analyte measurements and validated disease markers.

This matching process enables the data analyzing subsystem 210 to employ Template Matching Neural Networks that systematically scan the obtained analyte data to identify one or more analyte patterns such as specific combinations of elevated cytokine levels, viral RNA sequences, and protein concentrations, then compare these patterns against the one or more pre-defined patterns stored in the database by calculating cross-correlation coefficients, similarity indices, and pattern alignment scores using sliding window algorithms that can detect partial matches and account for biological variation while maintaining diagnostic specificity.

The data analyzing subsystem 210 further utilizes Siamese Neural Networks that learn to measure similarity between molecular patterns by processing pairs of analyte patterns through identical neural network architectures, where one network processes the current sample's molecular signature while the other processes each pre-defined pathogen pattern from the pre-stored analyte data, and the networks generate embedding vectors that are compared using distance metrics to determine how closely the obtained pattern matches each stored pathogen-specific template. The data analyzing subsystem 210 further implements Dynamic Time Warping Algorithms that can match temporal analyte patterns by aligning molecular concentration changes over time with stored progression patterns, allowing the system to identify pathogen signatures even when the timing or sequence of molecular changes varies from the standard template, using elastic matching techniques that can stretch or compress time series data to find optimal alignments between current and historical molecular patterns. These AI models work collaboratively where template matching networks identify static molecular signatures, Siamese networks quantify pattern similarity with high precision, and dynamic time warping handles temporal variations in disease progression, enabling comprehensive pattern recognition across diverse molecular features and time scales.

For example, when analyzing blood samples from a patient with suspected viral infection, the AI model extracts analyte patterns from the obtained analyte data showing elevated interleukin- 6 at 85 pg/mL combined with detected viral RNA sequences and increased interferon levels, then uses these three AI models to match this three-component molecular pattern against the pre-defined patterns in the pre-stored analyte data including COVID-19 Pattern Alpha (IL-6>70 pg/mL+SARS-CoV-2 RNA+interferon elevation) showing 96% template match, influenza Pattern Beta (IL-6>60 pg/mL+influenza RNA+interferon response) showing 78% Siamese similarity, and RSV Pattern Gamma (different cytokine profile+RSV RNA+modified immune response) showing 45% dynamic alignment, thereby identifying the patient's condition as COVID-19 based on the strongest pattern match with the pre-defined viral signature stored in the database.

In an embodiment, comparing the obtained analyte data with the pre-stored analyte data, to identify the one or more pathogens, using the AI model, the data analyzing subsystem 210 is further configured to identify one or more similarities and one or more differences, between current analyte profiles and historical analyte profiles. The process of identifying one or more similarities and one or more differences, between current analyte profiles and historical analyte profiles represents a comprehensive comparative analysis operation where the AI system systematically evaluates molecular measurement patterns from newly collected samples against previously documented molecular signatures from confirmed pathogen cases, utilizing advanced artificial intelligence algorithms to detect both matching characteristics that indicate known pathogen presence and divergent features that may suggest novel variants, emerging pathogens, or unique host responses.

This identification process enables the data analyzing subsystem 210 to employ Differential Analysis Neural Networks that systematically compare current analyte profiles containing fresh molecular measurements such as protein concentrations, genetic sequences, and biomarker levels from new samples with historical analyte profiles from the database by calculating correlation matrices, variance analyses, and statistical significance tests to identify similarities such as matching cytokine elevation patterns, identical viral RNA sequences, or comparable immune response markers that indicate the presence of known pathogens, while simultaneously detecting differences such as novel genetic mutations, altered protein expression levels, or unexpected biomarker combinations that may represent pathogen evolution or previously uncharacterized disease mechanisms.

The data analyzing subsystem 210 further utilizes Anomaly Detection Algorithms that learn normal patterns from historical analyte profiles and identify deviations in current profiles by establishing baseline molecular ranges, standard deviation thresholds, and expected correlation patterns from thousands of confirmed cases, then flagging current samples that exhibit unusual molecular signatures such as protein concentrations outside normal ranges, unexpected genetic variants, or atypical immune response patterns that differ significantly from established historical patterns.

The data analyzing subsystem 210 further utilizes Clustering and Classification Models that group similar analyte profiles together while identifying outliers and novel patterns by applying unsupervised learning techniques such as k-means clustering to organize historical profiles into distinct pathogen-specific groups, then evaluating where current profiles fit within these established clusters or whether they represent entirely new molecular signature categories that require further investigation. These AI models work synergistically where differential analysis networks quantify specific molecular similarities and differences, anomaly detection algorithms identify unusual patterns that deviate from historical norms, and clustering models provide context for understanding whether observed differences represent known variation or novel pathogen characteristics.

For example, when analyzing respiratory samples from patients during a flu outbreak, the AI model compares current analyte profiles showing elevated interleukin- 6 at 95 pg/mL, specific H3N2 hemagglutinin sequences, and interferon responses with historical analyte profiles from previous influenza seasons, identifying similarities including matching cytokine elevation patterns (IL-6 levels 80-100 pg/mL), identical core hemagglutinin genetic sequences, and comparable interferon response kinetics that confirm H3N2 influenza infection, while simultaneously detecting differences such as novel amino acid substitutions in the hemagglutinin binding domain, 15% higher peak cytokine concentrations than historical averages, and altered antibody binding patterns that suggest the emergence of a new H3N2 variant with potentially enhanced transmissibility or immune evasion capabilities compared to previously documented strains in the historical database.

In an embodiment, comparing the obtained analyte data with the pre-stored analyte data, to identify the one or more pathogens, using the AI model, the data analyzing subsystem 210 is further configured to determine correlation coefficients between the obtained analyte data and known pathogen signatures. The process of determining correlation coefficients between the obtained analyte data and known pathogen signatures represents a quantitative statistical analysis operation where the AI model calculates/determines numerical measures of linear and non-linear relationships between molecular measurements from current samples and established pathogen-specific molecular patterns, utilizing advanced mathematical algorithms to generate correlation values that indicate the strength and direction of associations between fresh molecular data and validated disease markers stored in the database.

The data analyzing subsystem 210 employs Pearson Correlation Neural Networks that systematically calculate/determine linear correlation coefficients between the obtained analyte data containing current molecular measurements such as protein concentrations, genetic expression levels, and biomarker values and known pathogen signatures consisting of established molecular patterns from confirmed cases, using mathematical formulas that generate correlation values ranging from −1 to +1 where values near +1 indicate strong positive relationships, values near −1 indicate strong negative relationships, and values near 0 suggest no linear association between current samples and stored pathogen patterns.

The data analyzing subsystem 210 further employs Spearman Rank Correlation Algorithms that calculate non-parametric correlation coefficients between the obtained analyte data and known pathogen signatures by converting molecular measurements to ranked positions rather than using raw numerical values, enabling detection of monotonic relationships even when the associations follow non-linear patterns such as exponential or logarithmic relationships commonly observed in biological systems where molecular concentrations may increase or decrease at varying rates during infection progression.

The data analyzing subsystem 210 further employs Canonical Correlation Analysis Models that simultaneously analyze multiple molecular variables to determine correlation coefficients between the obtained analyte data and known pathogen signatures by identifying which combinations of current molecular measurements correlate most strongly with specific disease patterns, finding optimal linear combinations of analyte variables that maximize correlation with corresponding combinations of pathogen signature variables. These AI models work synergistically where Pearson networks quantify linear molecular relationships, Spearman algorithms detect non-linear monotonic associations, and canonical correlation analysis reveals multi-dimensional relationships between current molecular profiles and stored pathogen patterns.

For example, when analyzing respiratory samples from patients with suspected influenza, the AI model applies these three algorithms to determine correlation coefficients between the obtained analyte data showing interferon levels at 125 pg/mL, viral RNA load at 10\hat{ }6 copies/mL, and inflammatory cytokine concentrations and known pathogen signatures for H3N2 influenza strain, calculating Pearson correlation coefficients of +0.87 between current interferon measurements and historical H3N2 immune response patterns, +0.94 between viral RNA levels and established influenza load signatures, and +0.82 between cytokine profiles and known H3N2 inflammatory markers, while Spearman analysis reveals +0.89 rank correlation for overall molecular progression patterns and canonical correlation analysis shows +0.93 correlation between the complete multi-variable analyte profile and the comprehensive H3N2 molecular signature, thereby providing quantitative mathematical evidence of strong positive correlations that confirm influenza infection based on statistical relationships between current molecular measurements and validated pathogen identification patterns.

In an embodiment, comparing the obtained analyte data with the pre-stored analyte data, to identify the one or more pathogens, using the AI model, the data analyzing subsystem 210 is further configured to determine one or more pattern matching scores for pathogen identification. The process of determining one or more pattern matching scores for pathogen identification represents a quantitative scoring operation where the AI model calculates/determines numerical values that measure how closely molecular patterns from current samples align with established pathogen-specific signatures, utilizing advanced computational algorithms to generate standardized scores that indicate the likelihood and confidence level of specific pathogen presence based on the degree of molecular pattern similarity and matching accuracy.

The determination process enables the data analyzing subsystem 210 to employ Template Matching Convolutional Neural Networks that systematically compare molecular patterns from the obtained analyte data against stored pathogen templates by sliding pattern recognition filters across nucleic acid sequences, protein concentration profiles, and biomarker combinations to calculate one or more pattern matching scores that quantify the degree of alignment between current molecular signatures and known pathogen patterns, generating scores typically ranging from 0 to 1 where higher values indicate stronger pattern matches and greater confidence in pathogen identification.

The data analyzing subsystem 210 further utilizes Dynamic Programming Alignment Algorithms that calculate optimal pattern matching scores by finding the best possible alignment between current analyte patterns and stored pathogen signatures using sequence alignment techniques, gap penalty functions, and substitution matrices that account for biological variation while maximizing similarity scores, enabling robust pathogen identification even when molecular patterns contain minor variations or measurement noise.

The data analyzing subsystem 210 further utilizes Ensemble Scoring Networks that combine multiple individual pattern matching algorithms to generate comprehensive pattern matching scores by aggregating results from different analytical approaches including sequence similarity measures, structural alignment scores, and statistical correlation indices, using weighted voting schemes and confidence intervals to produce final scores that reflect the consensus opinion of multiple analytical methods for reliable pathogen identification. These AI models work collaboratively where template matching networks identify specific molecular motifs and generate initial similarity scores, dynamic programming algorithms optimize alignment quality and calculate refined matching scores, and ensemble networks integrate multiple scoring approaches to produce robust final scores with associated confidence measures.

For example, when analyzing throat swab samples from patients with respiratory symptoms, the AI model applies these three approaches to determine pattern matching scores for pathogen identification by comparing obtained analyte data containing specific viral RNA sequences, elevated cytokine levels, and inflammatory biomarkers against stored pathogen signatures, calculating template matching scores of 0.94 for SARS-CoV-2 Omicron variant based on spike protein sequence alignment, 0.87 for influenza A H3N2 strain based on hemagglutinin pattern recognition, and 0.23 for respiratory syncytial virus based on limited molecular similarity, while dynamic programming alignment generates refined scores of 0.96, 0.82, and 0.19 respectively, and ensemble scoring produces final pattern matching scores of 0.95 for SARS-CoV-2, 0.84 for influenza A, and 0.21 for RSV, thereby enabling confident pathogen identification of COVID-19 infection based on the highest pattern matching score indicating strong molecular similarity between current sample patterns and established SARS-CoV-2 signatures.

For analyzing the obtained analyte data comprising the one or more analytes, to determine the biological status information, the data analyzing subsystem 210 is further configured to correlate one or more analyte patterns in the obtained analyte data, with known pathogen signatures, to determine pathogen-specific signatures within the obtained analyte data, using the AI model. The process of correlating one or more analyte patterns in the obtained analyte data, with known pathogen signatures, represents a sophisticated pattern correlation operation where the AI model systematically identifies and establishes relationships between characteristic molecular combinations found in current samples and validated pathogen-specific molecular templates (i.e., the known pathogen signatures) stored in the database, utilizing advanced computational algorithms to discover which specific molecular signatures within the fresh sample data correspond to particular disease-causing organisms.

The correlation process enables the data analyzing subsystem 210 to employ Cross-Correlation Neural Networks that systematically analyze one or more analyte patterns in the obtained analyte data such as specific combinations of elevated cytokine levels, viral RNA sequences, and protein concentrations by comparing these molecular arrangements with known pathogen signatures consisting of established disease-specific molecular templates, using sliding window correlation functions and pattern matching algorithms to identify regions of high similarity and determine pathogen-specific signatures within the obtained analyte data that match established disease markers.

The data analyzing subsystem 210 further utilizes Graph Neural Networks that represent molecular relationships as interconnected networks where nodes represent individual analytes and edges represent correlations between molecular measurements, enabling the system to correlate analyte patterns by analyzing network topology similarities between current sample molecular graphs and stored pathogen signature networks, identifying subgraph patterns that correspond to specific pathogens and thereby determining pathogen-specific signatures embedded within the complex molecular interaction networks of the current sample data.

The data analyzing subsystem 210 further utilizes Attention-Based Transformer Networks that use self-attention mechanisms to correlate analyte patterns by focusing on the most relevant molecular features and their relationships, learning which combinations of analytes are most indicative of specific pathogens through multi-head attention layers that can simultaneously analyze multiple molecular pattern correlations and identify the most significant pathogen-specific signatures within the obtained analyte data based on learned attention weights and feature importance scores. These AI models work synergistically where cross-correlation networks identify direct pattern similarities, graph neural networks reveal complex molecular interaction relationships, and attention-based transformers focus on the most diagnostically relevant molecular combinations for accurate pathogen signature identification.

For example, when analyzing blood samples from patients with suspected viral infection, the AI model applies these three approaches to correlate one or more analyte patterns in the obtained analyte data showing elevated interleukin- 6 at 89 pg/mL combined with specific viral RNA sequences and increased interferon levels with known pathogen signatures for various respiratory viruses, using cross-correlation analysis to identify 0.92 correlation with SARS-CoV-2 cytokine patterns, graph neural networks to detect molecular interaction networks matching COVID-19 immune response signatures, and attention mechanisms to focus on the most diagnostically significant three-analyte combination, thereby successfully determining pathogen-specific signatures within the obtained analyte data that correspond to SARS-CoV-2 Omicron variant infection based on the correlated molecular evidence and established pathogen signature matching.

In an embodiment, correlating the one or more analyte patterns in the obtained analyte data, with the known pathogen signatures, to determine pathogen-specific signatures within the obtained analyte data, using the AI model, the data analyzing subsystem 210 is further configured to map specific analyte combinations in the obtained analyte data to the known pathogen signatures. The process of mapping specific analyte combinations in the obtained analyte data to the known pathogen signatures, represents a systematic computational operation where the AI model creates direct correspondences and associations between particular molecular measurement groupings found in current samples and established pathogen-specific molecular templates (i.e., the known pathogen signatures) stored in the database, utilizing advanced algorithmic approaches to establish one-to-one or many-to-one relationships that link current molecular evidence to validated disease markers.

This mapping process enables the data analyzing subsystem 210 to employ Hierarchical Clustering Neural Networks that systematically organize and map specific analyte combinations in the obtained analyte data such as particular groupings of elevated cytokine levels, viral RNA sequences, and protein concentrations by creating hierarchical tree structures that connect these molecular combinations to the known pathogen signatures through similarity-based clustering algorithms, where the network learns to group similar molecular patterns together and establish mapping relationships between current sample combinations and stored pathogen templates based on molecular feature similarity and statistical correlation measures.

The data analyzing subsystem 210 further utilizes Self-Organizing Map Networks that create topological mappings by projecting high-dimensional analyte combinations from current samples onto lower-dimensional representation spaces where similar molecular patterns cluster together, enabling the data analyzing subsystem 210 to map current specific analyte combinations to known pathogen signatures by identifying which regions of the self-organizing map correspond to particular pathogens, using competitive learning algorithms where neurons compete to represent different molecular combinations and establish clear mapping relationships between input patterns and stored pathogen categories.

The data analyzing subsystem 210 further utilizes Bipartite Graph Matching Algorithms that create explicit mapping relationships by constructing bipartite graphs where one set of nodes represents specific analyte combinations in the obtained analyte data and the other set represents known pathogen signatures, using maximum weight matching algorithms and Hungarian optimization methods to find optimal one-to-one or many-to-one correspondences that maximize the overall similarity between current molecular combinations and stored pathogen patterns while minimizing mapping conflicts and ambiguities. These AI models work collaboratively where hierarchical clustering networks organize molecular patterns into meaningful groups, self-organizing maps create spatial representations of pattern relationships, and bipartite graph matching algorithms establish precise correspondence relationships between current and stored molecular signatures.

For example, when analyzing respiratory samples from patients with flu-like symptoms, the AI model applies these three approaches to map specific analyte combinations in the obtained analyte data including Combination A (interferon-gamma 145 pg/mL+H3N 2 hemagglutinin RNA+elevated neutrophil count), Combination B (interleukin-6 78 pg/mL+influenza nucleoprotein+specific antibody response), and Combination C (tumor necrosis factor 92 pg/mL+viral load 10\hat{ }5 copies/mL+inflammatory markers) to the known pathogen signatures for influenza A H3N2 strain, where hierarchical clustering maps Combination A to H3N 2 early infection signature with 0.91 similarity, self-organizing maps place Combination B in the H3N 2 moderate infection region with 0.87 correspondence, and bipartite graph matching establishes optimal mapping of Combination C to H3N 2 severe infection signature with 0.94 matching score, thereby successfully creating direct correspondences between current molecular evidence and established influenza pathogen patterns for accurate disease identification.

In an embodiment, correlating the one or more analyte patterns in the obtained analyte data, with the known pathogen signatures, to determine pathogen-specific signatures within the obtained analyte data, using the AI model, the data analyzing subsystem 210 is further configured to identify which of the one or more pathogens is potentially present based on analyte pattern matching. The process of identifying which of the one or more pathogens is potentially present based on analyte pattern matching represents a diagnostic classification operation where the AI model systematically evaluates molecular pattern similarities and correlations to determine the specific disease-causing organisms that are most likely present in the analyzed samples, utilizing advanced pattern recognition algorithms to distinguish between different pathogen types and make definitive identification decisions based on the strength and specificity of molecular pattern matches.

This identification process enables the data analyzing subsystem 210 to utilize Multi-Class Classification Neural Networks that systematically analyze molecular pattern matching results to identify which of the one or more pathogens including viruses, bacteria, fungi, and parasites is potentially present by processing pattern similarity scores, correlation coefficients, and molecular feature alignments based on analyte pattern matching results, using softmax activation functions and probability distributions to assign likelihood scores to each potential pathogen category and select the most probable pathogen identification based on the highest classification confidence scores.

The data analyzing subsystem 210 further utilizes Decision Tree Ensemble Models that create hierarchical decision pathways to identify which pathogen is potentially present by systematically evaluating molecular pattern matching criteria through branching logic structures, where each decision node represents a specific analyte pattern matching threshold or molecular feature comparison that guides the identification process toward increasingly specific pathogen categories, using random forest algorithms and gradient boosting methods to combine multiple decision trees and improve identification accuracy based on analyte pattern matching evidence from multiple molecular features simultaneously.

The data analyzing subsystem 210 further utilizes Bayesian Inference Networks that calculate posterior probabilities to identify which of the one or more pathogens is potentially present by combining prior knowledge about pathogen prevalence with current molecular evidence, using Bayes'theorem to update probability estimates based on analyte pattern matching results, where the network maintains probabilistic beliefs about different pathogen possibilities and continuously refines these beliefs as new molecular pattern matching evidence becomes available, ultimately selecting the pathogen with the highest posterior probability as the most likely identification. These AI models work synergistically where multi-class neural networks provide initial pathogen probability distributions, decision tree ensembles offer interpretable classification pathways, and Bayesian networks incorporate uncertainty quantification and prior knowledge to refine identification decisions.

For example, when analyzing blood samples from patients with systemic infection symptoms, the AI model applies these three approaches to identify which of the one or more pathogens is potentially present based on analyte pattern matching results showing strong molecular similarities with multiple pathogen signatures, where multi-class classification assigns 0.89 probability to Staphylococcus aureus, 0.76 probability to Streptococcus pneumoniae, and 0.23 probability to Escherichia coli based on protein pattern matches, decision tree analysis follows branching logic through gram-positive bacterial markers and coagulase-positive indicators to identify S. aureus as the most likely pathogen, and Bayesian inference combines these pattern matching results with prior infection prevalence data to conclude with 0.94 posterior probability that Staphylococcus aureus is the pathogen present in the analyzed samples, thereby providing definitive pathogen identification based on comprehensive analyte pattern matching analysis.

In an embodiment, correlating the one or more analyte patterns in the obtained analyte data, with the known pathogen signatures, to determine pathogen-specific signatures within the obtained analyte data, using the AI model, the data analyzing subsystem 210 is further configured to determine the pathogen-specific signatures within the obtained analyte data comprising the one or more analytes. The process of determining pathogen-specific signatures within the obtained analyte data comprising the one or more analytes, represents a signature identification operation where the AI model systematically analyzes and extracts characteristic molecular fingerprints that are uniquely associated with specific disease-causing organisms from the comprehensive molecular information contained within current sample measurements, utilizing advanced pattern recognition algorithms to isolate and define the distinctive combinations of molecular markers that serve as diagnostic indicators for particular pathogens.

This determination process enables the data analyzing subsystem 210 to utilize Feature Extraction Convolutional Neural Networks that systematically scan through the obtained analyte data comprising the one or more analytes including nucleic acid sequences, protein concentrations, metabolite levels, and biomarker profiles to identify and extract pathogen-specific signatures by applying learned convolutional filters that recognize characteristic molecular patterns such as unique viral RNA motifs, bacterial protein markers, or specific immune response profiles, using multiple layers of feature detection that progressively identify increasingly complex molecular signatures from basic sequence elements to complete pathogen-specific molecular fingerprints.

The data analyzing subsystem 210 further utilizes Principal Component Analysis Networks that analyze the high-dimensional obtained analyte data comprising the one or more analytes to determine pathogen-specific signatures by identifying the most significant molecular feature combinations that explain the greatest variance in the data, using dimensionality reduction techniques to isolate the key molecular components that distinguish different pathogen types, where principal components represent linear combinations of original analyte measurements that capture the essential pathogen-specific molecular characteristics while eliminating redundant or non-diagnostic information.

The data analyzing subsystem 210 further utilizes Autoencoder Neural Networks that learn to compress and reconstruct the obtained analyte data comprising the one or more analytes through encoder-decoder architectures, where the encoder learns to identify the most essential molecular features that represent pathogen-specific signatures by compressing complex analyte profiles into lower-dimensional latent representations, and the decoder attempts to reconstruct the original data, with the learned latent features representing the core pathogen-specific molecular signatures that capture the essential characteristics of different disease-causing organisms. These AI models work collaboratively where convolutional networks extract hierarchical molecular features, principal component analysis identifies the most discriminative molecular combinations, and autoencoders learn compressed representations of pathogen-specific molecular signatures.

For example, when analyzing respiratory samples from patients with pneumonia symptoms, the AI model applies these three approaches to determine pathogen-specific signatures within the obtained analyte data comprising the one or more analytes including elevated white blood cell counts, specific bacterial DNA sequences, inflammatory cytokine levels, and metabolic byproducts, where convolutional networks extract characteristic Streptococcus pneumoniae genetic motifs and associated immune response patterns, principal component analysis identifies the three-component signature consisting of pneumococcal surface protein detection plus interleukin-8 elevation plus specific metabolite profiles that explains 87% of the variance distinguishing pneumococcal from other bacterial infections, and autoencoder networks learn a compressed five-dimensional latent representation that captures the essential pneumococcal molecular fingerprint, thereby successfully determining the pathogen-specific signature that uniquely identifies Streptococcus pneumoniae infection within the complex molecular landscape of the analyzed sample data.

For analyzing the obtained analyte data comprising the one or more analytes, to determine the biological status information, the data analyzing subsystem 210 is further configured to extract receptor binding characteristics from the obtained analyte data, using the AI model, by: (a) identifying receptor binding proteins and concentrations of the receptor binding proteins, within the obtained analyte data; (b) analyzing three dimensional protein structures in the obtained analyte data for determining binding domain compatibility; (c) evaluating ligand receptor binding profiles present in the obtained analyte data; and (d) generating one or more scores for binding affinity potential based on molecular interaction data within the obtained analyte data. The process of extracting the receptor binding characteristics from the obtained analyte data, using the AI model, represents a comprehensive molecular interaction analysis operation where the AI model systematically identifies, analyzes, and quantifies the specific molecular features related to how pathogens interact with host cell receptors by examining protein structures, binding affinities, and molecular interaction patterns contained within the current sample data, utilizing advanced computational biology algorithms to extract critical information about pathogen-host cellular attachment mechanisms that determine infection potential and disease severity. In other words, the receptor binding characteristics are the specific molecular properties, affinities, and interaction profiles that describe how pathogens or their components (such as viral proteins, bacterial toxins, or other pathogenic molecules) physically attach to, interact with, and bind to specific receptor proteins or binding sites on host cell surfaces, including binding affinity strength (measured by dissociation constants like Kd values), binding specificity (selectivity for particular receptor types), binding kinetics (association and dissociation rates), structural compatibility between pathogen ligands and host receptors, conformational changes induced upon binding, competitive binding relationships with natural ligands, and the resulting cellular entry mechanisms, signal transduction pathways, or biological responses that occur following receptor-pathogen binding interactions, which collectively determine the pathogen's ability to infect specific cell types, tissues, or host species and influence disease severity, transmission potential, and therapeutic intervention strategies.

The receptor binding proteins are specific cellular surface or intracellular proteins that serve as attachment points for pathogens, including molecules such as ACE2 receptors, CD4 receptors, integrin proteins, and other membrane-bound or cytoplasmic proteins that pathogens use to gain entry into host cells or initiate infection processes. The concentrations of the receptor binding proteins refer to the quantitative measurements of these specific proteins expressed as numerical values with appropriate units such as ng/mL, μg/mL, or molecules per cell, indicating the abundance or density of receptor proteins available for pathogen binding within the analyzed sample.

This extraction process involves four integrated analytical steps where the AI model first performs identifying receptor binding proteins and concentrations of the receptor binding proteins, within the obtained analyte data, using Protein Classification Neural Networks that scan molecular data to recognize specific receptor proteins such as ACE2, CD4, or integrin receptors along with their measured concentrations, applying learned protein sequence patterns and structural motifs to distinguish receptor proteins from other cellular proteins and quantify their abundance levels in the sample.

The three dimensional protein structures are the spatial arrangements and folding patterns of proteins in three-dimensional space, including primary amino acid sequences, secondary structures such as alpha helices and beta sheets, tertiary folding configurations, and quaternary multi-subunit assemblies that determine protein function and binding capabilities. The binding domain compatibility represents the geometric and chemical complementarity between pathogen surface proteins and host receptor binding sites, measuring how well the molecular shapes, electrostatic charges, and chemical properties of pathogen and host proteins fit together to enable successful molecular interaction and attachment.

The AI model conducts analyzing three dimensional protein structures in the obtained analyte data for determining binding domain compatibility, using 3D Structural Analysis Convolutional Networks that process protein crystallography data, NMR structures, and predicted protein folding patterns to evaluate how pathogen surface proteins can physically interact with host receptor binding sites, calculating geometric compatibility scores, surface complementarity measures, and electrostatic interaction potentials that indicate whether pathogen proteins can successfully dock with host cellular receptors.

The ligand receptor binding profiles are comprehensive datasets describing the interaction characteristics between pathogen molecules (ligands) and host cell receptors, including binding kinetics, association and dissociation rates, equilibrium constants, competitive binding data, and thermodynamic parameters that characterize the strength and specificity of molecular interactions.

Subsequently, the data analyzing subsystem 210 evaluates ligand receptor binding profiles present in the obtained analyte data, using Molecular Docking Simulation Networks that model the thermodynamic and kinetic aspects of pathogen-receptor interactions by analyzing binding kinetics data, dissociation constants, and competitive binding assays contained in the analyte data, using machine learning algorithms trained on extensive protein-protein interaction databases to predict binding strength, selectivity, and stability of pathogen-receptor complexes.

The binding affinity potential refers to the predicted or measured strength of attraction between pathogen molecules and host cell receptors, typically expressed as dissociation constants, binding energies, or probability scores that indicate how likely and how strongly a pathogen can attach to and interact with specific host cellular targets. The Molecular interaction data encompasses all quantitative and qualitative information about how molecules interact with each other, including binding affinities, interaction energies, contact surfaces, hydrogen bonding patterns, electrostatic interactions, and other physical and chemical parameters that describe molecular recognition and binding events.

Finally, the data analyzing subsystem 210 generates one or more scores for binding affinity potential based on molecular interaction data within the obtained

analyte data using Multi-Modal Fusion Neural Networks that integrate results from protein identification, structural analysis, and binding profile evaluation through attention mechanisms and weighted feature combination layers to produce quantitative scores representing the likelihood and strength of pathogen-receptor interactions, employing ensemble scoring algorithms that combine multiple molecular evidence sources including protein concentration data, structural compatibility measures, and binding kinetics parameters to generate comprehensive binding affinity predictions with associated confidence intervals and uncertainty estimates.

For example, when analyzing blood samples from COVID-19 patients, the AI model extracts receptor binding characteristics from the obtained analyte data by first identifying receptor binding proteins including ACE2 receptors at 45 ng/mL concentration and TMPRSS2 protease at 23 ng/mL within the sample data, then analyzing three dimensional protein structures of SARS-CoV-2 spike protein domains to determine 0.89 binding domain compatibility with ACE2 receptor binding sites based on structural complementarity analysis, subsequently evaluating ligand receptor binding profiles showing high-affinity binding kinetics with dissociation constant of 14.7 nM and competitive binding scores indicating strong receptor occupancy potential, and finally generating one or more scores for binding affinity potential of 0.92 for spike-ACE2 interaction strength, 0.87 for membrane fusion potential, and 0.84 for overall cellular entry capability using Multi-Modal Fusion Neural Networks that integrate all molecular evidence sources, thereby providing comprehensive molecular evidence of the pathogen's ability to successfully bind to and enter host cells based on quantitative analysis of receptor binding characteristics extracted from the obtained analyte data.

For analyzing the obtained analyte data comprising the one or more analytes, to determine the biological status information, the data analyzing subsystem 210 is further configured to determine host cell adherence capability from the obtained analyte data, using the AI model, by: (a) detecting adhesion protein markers and concentrations in the obtained analyte data; (b) analyzing protein-protein interaction profiles within the obtained analyte data for cell surface binding; (c) identifying attachment mechanism indicators present in the obtained analyte data; and (d) quantifying potential host cell adherence using molecular signatures found in the obtained analyte data.

The host cell adherence capability refers to the pathogen's ability to attach to and bind with host cell surfaces through specific molecular mechanisms, representing the strength and efficiency with which disease-causing organisms can establish initial contact and maintain stable attachment to target cells during the infection process. The adhesion protein markers are specific proteins produced by pathogens that facilitate attachment to host cells, including molecules such as adhesins, fimbriae, pili, surface glycoproteins, and other cell wall or membrane proteins that enable pathogens to recognize and bind to specific host cell receptors or surface structures. The concentrations represent the quantitative measurements of adhesion protein markers expressed as numerical values with appropriate units such as ng/mL, μg/mL, or copy numbers per sample, indicating the abundance of adhesion-related proteins present in the analyzed sample.

The process of detecting adhesion protein markers and concentrations in the obtained analyte data is implemented through Protein Detection Convolutional Neural Networks that systematically scan and analyze the molecular information within current sample data to identify specific pathogen-produced proteins responsible for cellular attachment and quantify their abundance levels using advanced pattern recognition and quantification algorithms. This detection process operates through a multi-stage computational pipeline where the Protein Detection Convolutional Neural Network first applies learned convolutional filters trained on extensive protein sequence databases to scan through the obtained analyte data containing mass spectrometry results, protein expression profiles, and molecular concentration measurements, using sliding window algorithms that move across the data to identify characteristic amino acid sequences, protein structural motifs, and molecular weight signatures that correspond to known adhesion protein markers such as bacterial adhesins, viral attachment proteins, fungal cell wall proteins, and parasitic surface glycoproteins. The network then employs specialized quantification layers that process detected protein signals through regression algorithms and calibration curves to determine the precise concentrations of identified adhesion proteins, using reference standards and internal controls to convert raw signal intensities into standardized concentration units such as ng/mL, μg/mL, or molecules per sample volume. The Protein Detection Convolutional Neural Network utilizes multiple convolutional layers with different filter sizes to capture protein features at various scales, from individual amino acid patterns to complete protein domains, followed by pooling layers that reduce computational complexity while preserving critical protein identification information, and fully connected layers that integrate all detected features to make final protein identification and quantification decisions with associated confidence scores. The network architecture includes attention mechanisms that focus on the most diagnostically relevant protein regions, batch normalization layers that ensure stable training across diverse sample types, and dropout regularization that prevents overfitting to specific protein variants while maintaining generalization capability across different pathogen species and strains.

For example, when analyzing respiratory samples from patients with bacterial pneumonia, the Protein Detection Convolutional Neural Network systematically processes the obtained analyte data containing complex protein mixture information to detect adhesion protein markers and concentrations by first applying convolutional filters that recognize Streptococcus pneumoniae pneumococcal surface protein A (PspA) sequence patterns and structural signatures within the mass spectrometry data, then using quantification algorithms to determine PspA concentration at 67 ng/mL, simultaneously detecting pneumococcal adherence and virulence factor A (PavA) at 34 ng/mL and choline-binding protein A (CbpA) at 52 ng/mL, while also identifying Haemophilus influenzae adhesin HMW1 at 23 ng/mL and outer membrane protein P2 at 18 ng/mL, thereby providing comprehensive detection and quantification of multiple bacterial adhesion proteins that indicate the presence of specific respiratory pathogens and their relative abundance levels within the analyzed sample.

The protein-protein interaction profiles are comprehensive datasets describing how pathogen adhesion proteins interact with host cell surface proteins, including binding specificities, interaction networks, contact interfaces, and molecular recognition patterns that facilitate cellular attachment. The Cell surface binding refers to the specific molecular interactions between pathogen adhesion proteins and host cell membrane receptors, glycoproteins, or other surface molecules that enable pathogen attachment to the exterior of target cells.

The process of analyzing protein-protein interaction profiles within the obtained analyte data for cell surface binding is implemented through Graph Convolutional Neural Networks that systematically examine and evaluate the complex molecular interaction networks between pathogen adhesion proteins and host cell surface receptors by representing protein interactions as graph structures and applying specialized graph-based learning algorithms to understand binding relationships, interaction strengths, and cellular attachment mechanisms. This analysis process operates through a sophisticated graph-based computational framework where the Graph Convolutional Neural Network first constructs molecular interaction graphs from the obtained analyte data by representing individual proteins as nodes and their interactions as weighted edges, where pathogen adhesion proteins such as bacterial fimbriae, viral spike proteins, or fungal cell wall components are connected to host cell surface receptors like integrins, cadherins, or glycoprotein receptors through edges that encode interaction strength, binding affinity, and contact probability based on experimental binding assay data, co-immunoprecipitation results, and molecular docking predictions contained within the sample data. The network then applies multiple graph convolutional layers that propagate information across the protein interaction network, where each layer updates node representations by aggregating information from neighboring proteins and their interaction strengths, enabling the system to learn complex protein-protein interaction profiles that capture not only direct binding relationships but also indirect effects, cooperative binding events, and competitive interactions that influence cell surface binding efficiency and specificity. The Graph Convolutional Neural Network employs attention mechanisms that focus on the most critical protein interactions for cellular attachment, using learned attention weights to emphasize binding pairs that contribute most significantly to pathogen adherence while de-emphasizing less relevant molecular interactions, and incorporates temporal dynamics modules that can analyze how protein interaction profiles change over time during infection progression or in response to treatment interventions. The network architecture includes specialized pooling operations that aggregate interaction information across different protein families and binding domains, normalization layers that ensure stable learning across diverse protein interaction scales and affinities, and readout functions that translate complex graph representations into interpretable binding strength scores, attachment probability measures, and cellular entry potential assessments.

For example, when analyzing blood samples from patients with Staphylococcus aureus infection, the Graph Convolutional Neural Network processes the obtained analyte data containing protein interaction assay results, binding kinetics measurements, and cellular attachment studies to analyze protein-protein interaction profiles within the obtained analyte data for cell surface binding by first constructing an interaction graph where S. aureus fibronectin-binding proteins (FnBPs) are connected to host fibronectin receptors with edge weights representing binding affinities of 2.3×10\hat{ }−8 M, clumping factor A (ClfA) nodes are linked to fibrinogen receptor nodes with interaction strengths of 1.7×10\hat{ }−7 M, and protein A nodes connect to immunoglobulin Fc receptor nodes with binding constants of 4.2×10\hat{ }−9 M, then applying graph convolutional operations that reveal cooperative binding effects where FnBP-fibronectin interactions enhance ClfA-fibrinogen binding by 34% and competitive effects where protein A binding reduces available Fc receptors for other interactions by 28%, ultimately generating comprehensive protein-protein interaction profiles that show S. aureus achieves 0.89 overall cell surface binding efficiency through coordinated multi-protein attachment mechanisms involving synergistic adhesin-receptor interactions and strategic receptor competition patterns.

The attachment mechanism indicators are molecular signatures, protein expressions, or biochemical markers that reveal the specific biological pathways and molecular processes through which pathogens establish and maintain adherence to host cells.

The process of identifying attachment mechanism indicators present in the obtained analyte data is implemented through Recurrent Neural Networks with Long Short-Term Memory (LSTM-RNN) that systematically examine temporal and sequential molecular patterns within current sample data to recognize specific biochemical signatures, gene expression cascades, and metabolic pathway activations that reveal the underlying biological mechanisms through which pathogens establish and maintain cellular attachment. This identification process operates through a sophisticated sequential analysis framework where the LSTM-RNN processes the obtained analyte data as time-series molecular information, analyzing how protein expressions, gene activations, and metabolic markers change over time during pathogen attachment phases, using memory cells and gating mechanisms to capture long-term dependencies between early attachment initiation signals and later adhesion stabilization markers that collectively indicate specific attachment mechanism indicators. The LSTM-RNN architecture employs input gates that selectively process relevant molecular signals from the analyte data while filtering out noise and irrelevant biological background, forget gates that determine which previously observed molecular patterns should be retained or discarded as new attachment evidence emerges, and output gates that decide which internal molecular representations should be used to identify specific attachment mechanisms based on learned patterns from extensive training datasets containing confirmed pathogen attachment scenarios. The network utilizes specialized attention mechanisms that focus on critical temporal windows during attachment processes, identifying key molecular events such as initial pathogen-host contact signaling, adhesin protein upregulation phases, host cell receptor clustering events, and cytoskeletal rearrangement cascades that collectively represent distinct attachment mechanism indicators present in the obtained analyte data. The LSTM-RNN incorporates bidirectional processing capabilities that analyze molecular sequences both forward and backward in time to capture complete attachment mechanism signatures, ensemble learning components that combine multiple LSTM networks trained on different molecular feature types, and hierarchical processing layers that identify attachment indicators at multiple biological scales from individual protein interactions to complete cellular pathway activations.

For example, when analyzing epithelial cell samples from patients with enteropathogenic E. coli infection, the LSTM-RNN processes the obtained analyte data containing time-course gene expression profiles, protein phosphorylation cascades, and metabolic flux measurements to identify attachment mechanism indicators present in the obtained analyte data by first detecting early-phase molecular signatures including Tir protein expression upregulation at 15 minutes post-contact, intimin receptor clustering signals at 25 minutes, and actin polymerization markers at 35 minutes that indicate Type III secretion system-mediated attachment initiation, then identifying mid-phase indicators including pedestal formation proteins, cytoskeletal rearrangement enzymes, and tight junction disruption markers that reveal attaching and effacing lesion formation mechanisms, and finally recognizing late-phase stabilization indicators including persistent adhesion protein maintenance, host cell survival pathway modulation, and bacterial microcolony formation signals, thereby comprehensively identifying the complete temporal sequence of attachment mechanism indicators that characterize enteropathogenic E. coli's distinctive attaching and effacing pathogenesis mechanism based on sequential molecular evidence patterns extracted from the time-resolved analyte data.

The Potential host cell adherence represents the predicted or measured likelihood and strength of pathogen attachment to host cells based on molecular evidence and analytical results. The molecular signatures are characteristic patterns of molecular markers, protein expressions, gene expressions, or metabolite profiles that collectively indicate specific biological processes or pathogen behaviors related to cellular attachment and adherence.

The process of quantifying potential host cell adherence using molecular signatures found in the obtained analyte data is implemented through Gradient Boosting Regression Neural Networks that systematically integrate and quantify multiple molecular evidence sources to generate numerical scores representing the likelihood and strength of pathogen attachment to host cells, utilizing ensemble learning algorithms that combine weak predictive models into powerful quantification systems capable of producing accurate adherence probability estimates with associated confidence intervals. This quantification process operates through a sophisticated iterative learning framework where the Gradient Boosting Regression Neural Network sequentially builds multiple decision tree models that each focus on different aspects of molecular signatures found in the obtained analyte data, including adhesion protein concentrations, binding affinity measurements, attachment mechanism pathway activations, and host cell receptor availability indicators, using gradient descent optimization to minimize prediction errors by having each successive model learn from the residual errors of previous models until achieving optimal quantification of potential host cell adherence. The Gradient Boosting Regression Neural Network employs feature importance ranking algorithms that automatically identify which molecular signatures contribute most significantly to adherence prediction, assigning higher weights to critical molecular indicators such as high-affinity adhesin concentrations, activated attachment pathway markers, and favorable host receptor expression levels while reducing the influence of less predictive molecular features, enabling the system to focus computational resources on the most diagnostically relevant molecular evidence for accurate adherence quantification. The network architecture incorporates regularization techniques including learning rate control, tree depth limitations, and early stopping mechanisms that prevent overfitting to specific molecular signature patterns while maintaining generalization capability across diverse pathogen types and host cell varieties, and utilizes cross-validation procedures that ensure robust quantification performance across different sample types and clinical conditions. The Gradient Boosting Regression Neural Network implements uncertainty quantification modules that generate confidence intervals and prediction reliability scores alongside adherence estimates, providing clinicians and researchers with both point estimates and uncertainty measures that indicate the reliability of adherence predictions based on the quality and completeness of available molecular signature evidence.

For example, when analyzing urogenital samples from patients with Candida albicans infection, the Gradient Boosting Regression Neural Network processes the obtained analyte data containing comprehensive molecular information to quantify potential host cell adherence using molecular signatures found in the obtained analyte data by first identifying key molecular signatures including Als3 adhesin protein at 89 ng/mL, Hwp1 hyphal wall protein at 67 ng/mL, activated MAP kinase pathway markers, elevated cAMP signaling indicators, and host epithelial cell integrin receptor upregulation signals, then applying iterative gradient boosting algorithms where the first decision tree model achieves 0.73 adherence prediction accuracy based on Als3 concentrations alone, the second model improves accuracy to 0.84 by incorporating Hwp1 levels and correcting first model errors, the third model reaches 0.91 accuracy by adding pathway activation data, and subsequent models incrementally improve performance to final accuracy of 0.96, ultimately generating quantified potential host cell adherence scores of 0.92 for vaginal epithelium attachment probability, 0.87 for urethral binding likelihood, and 0.89 for overall urogenital colonization potential with confidence intervals of ±0.04, thereby providing precise numerical estimates of C. albicans adherence capability based on comprehensive integration of multiple molecular signature evidence sources from the analyzed sample data.

For analyzing the obtained analyte data comprising the one or more analytes, to determine the biological status information, the data analyzing subsystem 210 is further configured to identify one or more virulence genetic factors comprising at least one of: virulence gene sequences, toxin-encoding genetic elements, immune evasion markers, pathogenicity indicators, in the obtained analyte data, using the AI model. The virulence genetic factors are specific DNA sequences, genes, or genetic elements that encode proteins or regulatory molecules responsible for a pathogen's ability to cause disease, establish infection, evade host defenses, and produce harmful effects in host organisms. The virulence gene sequences are specific DNA or RNA sequences that code for proteins directly involved in pathogenesis, including genes that produce toxins, adhesins, invasins, immune evasion proteins, and other molecules that enhance the pathogen's ability to infect, colonize, and damage host tissues. The toxin-encoding genetic elements are DNA sequences, genes, or genetic cassettes that contain the coding information for producing toxic substances such as exotoxins, endotoxins, cytotoxins, neurotoxins, or other harmful compounds that directly damage host cells or disrupt normal physiological functions.

The immune evasion markers are genetic sequences that encode proteins or regulatory elements enabling pathogens to avoid, suppress, or circumvent host immune responses, including genes for antigenic variation, complement resistance, antibody degradation, immune cell inhibition, and molecular mimicry mechanisms. The pathogenicity indicators are genetic markers, sequences, or elements that correlate with or predict a pathogen's disease-causing potential, including virulence-associated genes, pathogenicity islands, mobile genetic elements carrying virulence factors, and regulatory sequences controlling virulence gene expression.

The process of identifying one or more virulence genetic factors comprising at least one of: virulence gene sequences, toxin-encoding genetic elements, immune evasion markers, pathogenicity indicators, in the obtained analyte data, using the AI model is implemented through Transformer-Based Sequence Analysis Neural Networks that systematically scan, analyze, and classify genetic sequences within current sample data to recognize specific DNA/RNA patterns associated with pathogen virulence and disease-causing capabilities. This identification process operates through a sophisticated multi-head attention mechanism where the Transformer Neural Network first tokenizes genetic sequence data from the obtained analyte data by converting nucleotide sequences into numerical representations that can be processed by the neural network, using positional encoding to maintain sequence order information and embedding layers that map genetic tokens into high-dimensional feature spaces where similar genetic patterns cluster together based on functional similarity and evolutionary relationships. The network then applies multiple self-attention layers that simultaneously examine all positions within genetic sequences to identify virulence gene sequences by learning which nucleotide combinations and gene structures correspond to known virulence factors, using attention weights to focus on critical genetic regions such as promoter sequences, coding regions, and regulatory elements that control virulence gene expression and protein production. The Transformer Neural Network employs specialized classification heads that process attention-weighted sequence representations to identify toxin-encoding genetic elements through pattern recognition algorithms trained on extensive databases of confirmed toxin genes, using learned sequence motifs, codon usage patterns, and structural gene features to distinguish toxin-encoding sequences from housekeeping genes and other non-virulent genetic elements. The network incorporates additional attention mechanisms specifically designed to detect immune evasion markers by recognizing genetic signatures associated with antigenic variation systems, complement resistance genes, and immune suppression factors, using comparative genomics approaches that identify genetic elements commonly found in immune-evasive pathogens but absent in non-pathogenic organisms. Finally, the system utilizes ensemble classification modules to identify pathogenicity indicators by integrating evidence from virulence gene detection, toxin identification, and immune evasion marker recognition to generate comprehensive pathogenicity assessments that consider the complete genetic virulence profile rather than individual genetic elements in isolation.

When analyzing bacterial isolates from patients with severe pneumonia, the Transformer-Based Sequence Analysis Neural Network processes the obtained analyte data containing whole genome sequencing results and metagenomic data to identify one or more virulence genetic factors by first detecting virulence gene sequences including Streptococcus pneumoniae pneumolysin gene (ply) with 98% sequence identity to reference virulence databases, autolysin gene (lytA) showing characteristic cell wall hydrolase coding patterns, and neuraminidase gene (nanA) containing conserved enzymatic domains associated with tissue invasion, then identifying toxin-encoding genetic elements including the pneumolysin toxin gene cluster with complete coding sequences for pore-forming cytotoxin production, hydrogen peroxide production genes (spxB) encoding toxic metabolite synthesis, and capsular polysaccharide synthesis genes that produce anti-phagocytic toxins, subsequently recognizing immune evasion markers including pneumococcal surface protein A (pspA) genes that encode complement-binding proteins, IgA1 protease gene (iga) for antibody degradation, and phase variation systems for antigenic switching, and finally detecting pathogenicity indicators including the complete pathogenicity island containing coordinated virulence gene clusters, mobile genetic elements carrying antibiotic resistance linked to virulence factors, and regulatory sequences controlling virulence gene expression under host-specific conditions, thereby comprehensively identifying the complete genetic virulence arsenal that enables S. pneumoniae to cause severe invasive disease through coordinated toxin production, immune evasion, and tissue invasion mechanisms based on systematic analysis of genetic virulence factors present in the obtained analyte data.

For analyzing the obtained analyte data comprising the one or more analytes, to determine the biological status information, the data analyzing subsystem 210 is further configured to classify the one or more pathogens comprising potential zoonotic pathogens from the obtained analyte data, using the AI model, by: (a) comparing the pathogen-specific signatures within the obtained analyte data against known zoonotic pathogen patterns; (b) identifying species-jumping genetic markers within the obtained analyte data; (c) detecting cross-species transmission indicators present in the obtained analyte data; and (d) classifying the potential zoonotic pathogens based on one or more patterns associated with the potential zoonotic pathogens determined in the obtained analyte data.

The potential zoonotic pathogens are disease-causing microorganisms that have the capability or likelihood to transmit from animals to humans or between different animal species, representing infectious agents that pose cross-species transmission risks and potential pandemic threats. The pathogen-specific signatures are unique molecular fingerprints, genetic patterns, or biochemical characteristics that distinctly identify particular disease-causing organisms, including specific DNA sequences, protein profiles, metabolic markers, or antigenic patterns that serve as diagnostic identifiers for individual pathogen species or strains. The Known zoonotic pathogen patterns are established molecular templates, genetic signatures, or biochemical profiles from previously confirmed zoonotic pathogens that have been validated through epidemiological studies and stored in reference databases for comparative analysis and identification purposes.

The process of classifying the one or more pathogens comprising potential zoonotic pathogens from the obtained analyte data, using the AI model is implemented through Hierarchical Multi-Task Classification Neural Networks that systematically analyze molecular data through four integrated classification stages to identify and categorize pathogens with zoonotic transmission potential. The first stage involves comparing the pathogen-specific signatures within the obtained analyte data against known zoonotic pathogen patterns where the network employs Siamese Convolutional Neural Networks that process pairs of molecular signatures by feeding pathogen-specific signatures extracted from current samples through one branch of identical neural networks while processing known zoonotic pathogen patterns from reference databases through the parallel branch, using contrastive learning algorithms that calculate similarity scores between current pathogen signatures and established zoonotic patterns, applying learned feature representations that capture genetic sequence similarities, protein structure homologies, and metabolic pathway correspondences to generate quantitative matching scores that indicate how closely current pathogens resemble confirmed zoonotic agents.

The species-jumping genetic markers are specific DNA sequences, genes, or genetic elements that enable pathogens to adapt to and infect new host species, including receptor binding domain mutations, host range expansion genes, and adaptive genetic changes that facilitate cross-species transmission. The data analyzing subsystem 210 implements identifying species-jumping genetic markers within the obtained analyte data using Attention-Based Transformer Networks that scan genetic sequences to recognize specific species-jumping genetic markers including receptor binding domain mutations, host range expansion genes, and adaptive genetic elements, employing multi-head attention mechanisms that simultaneously examine multiple genetic loci associated with host adaptation, using positional encoding to maintain genetic sequence context and learned attention weights that focus on critical genetic regions known to facilitate cross-species transmission, generating identification scores for genetic markers that enable pathogen adaptation to new host species.

The cross-species transmission indicators are molecular signatures, genetic features, or biochemical markers that suggest a pathogen's ability to move between different animal species or from animals to humans, including host adaptation markers, receptor binding versatility, and immune evasion mechanisms effective across multiple species. The data analyzing subsystem 210 executes detecting cross-species transmission indicators present in the obtained analyte data through Graph Neural Networks that model complex molecular interaction networks to identify cross-species transmission indicators including host receptor binding versatility, immune evasion mechanisms effective across multiple species, and metabolic adaptability markers, representing molecular interactions as graph structures where nodes represent individual molecular features and edges represent functional relationships, applying graph convolutional operations that propagate information across molecular networks to detect patterns indicative of cross-species transmission capability.

The one or more patterns associated with the potential zoonotic pathogens are characteristic combinations of molecular signatures, genetic features, and biochemical markers that collectively indicate zoonotic transmission potential, including multi-gene signatures, protein expression profiles, and metabolic patterns that distinguish zoonotic from non-zoonotic pathogens. The data analyzing subsystem 210 performs classifying the potential zoonotic pathogens based on one or more patterns associated with the potential zoonotic pathogens determined in the obtained analyte data using Ensemble Classification Networks that integrate evidence from signature comparison, genetic marker identification, and transmission indicator detection to generate final zoonotic classification decisions, employing voting mechanisms that combine predictions from multiple specialized classifiers, uncertainty quantification modules that provide confidence estimates, and hierarchical decision trees that categorize pathogens into specific zoonotic risk categories based on comprehensive molecular evidence analysis.

For example, when analyzing respiratory samples from bats in a wildlife surveillance program, the Hierarchical Multi-Task Classification Neural Network processes the obtained analyte data containing viral genetic sequences, protein profiles, and metabolic markers to classify potential zoonotic pathogens by first comparing pathogen-specific signatures including novel coronavirus spike protein sequences, RNA polymerase genes, and nucleocapsid protein patterns against known zoonotic pathogen patterns from SARS-CoV, MERS-CoV, and other confirmed zoonotic coronaviruses, generating similarity scores of 0.78 for spike protein homology, 0.85 for polymerase conservation, and 0.72 for nucleocapsid similarity that indicate significant resemblance to established zoonotic coronavirus patterns, then identifying species-jumping genetic markers including receptor binding domain mutations that enable binding to both bat ACE2 and human ACE2 receptors, furin cleavage site insertions that enhance viral entry across species barriers, and host range expansion genes showing 0.89 similarity to known cross-species adaptation markers, subsequently detecting cross-species transmission indicators including broad receptor binding specificity markers, immune evasion proteins effective against both bat and human immune systems, and metabolic flexibility indicators that suggest survival across different host environments with detection confidence scores of 0.91 for receptor versatility and 0.87 for immune evasion capability, and finally classifying the potential zoonotic pathogens based on patterns that combine high similarity to known zoonotic coronaviruses, presence of species-jumping genetic markers, and strong cross-species transmission indicators to generate a final classification of “High Zoonotic Risk Coronavirus” with 0.94 confidence, thereby identifying a novel bat coronavirus with significant pandemic potential based on comprehensive molecular pattern analysis that reveals strong similarities to previously emerged zoonotic pathogens and genetic features associated with successful cross-species transmission.

For analyzing the obtained analyte data comprising the one or more analytes, to determine the biological status information, the data analyzing subsystem 210 is further configured to assign one or more pandemic threat scores to the potential zoonotic pathogens based on at least one of: the receptor binding characteristics, the host cell adherence capability, and the one or more virulence genetic factors, using the AI model. The one or more pandemic threat scores are quantitative numerical values ranging from 0 to 1 or scaled scoring systems that represent the assessed risk level and potential for a zoonotic pathogen to cause widespread disease outbreaks across multiple populations, countries, or continents, with higher scores indicating greater pandemic potential.

The process of assigning the one or more pandemic threat scores to the potential zoonotic pathogens based on at least one of: the receptor binding characteristics, the host cell adherence capability, and the one or more virulence genetic factors, using the AI model, is implemented through Multi-Criteria Decision Analysis Neural Networks that systematically integrate and weight multiple risk factors to generate comprehensive pandemic threat assessments. The network employs Weighted Feature Fusion Layers that combine receptor binding characteristics including binding affinity scores, structural compatibility measures, and cellular entry potential with host cell adherence capability scores encompassing adhesion protein concentrations, attachment mechanism strength, and cellular binding efficiency, along with virulence genetic factors including toxin production potential, immune evasion capabilities, and pathogenicity indicators, using learned weight matrices that reflect the relative importance of each factor in determining pandemic risk based on historical pandemic data and epidemiological evidence. The Multi-Criteria Decision Analysis Neural Network applies Risk Aggregation Algorithms that process the integrated molecular evidence through non-linear transformation functions, normalization procedures that ensure different measurement scales are appropriately combined, and ensemble scoring methods that generate robust pandemic threat scores with associated uncertainty estimates and confidence intervals that reflect the reliability of available molecular evidence.

For example, when analyzing a novel influenza strain isolated from poultry, the Multi-Criteria Decision Analysis Neural Network processes previously extracted molecular evidence to assign pandemic threat scores to the potential zoonotic pathogen by integrating receptor binding characteristics showing high-affinity binding to both avian α-2,3 and human α-2,6 sialic acid receptors with binding scores of 0.89 and 0.76 respectively, host cell adherence capability demonstrating strong attachment to respiratory epithelial cells with adhesion scores of 0.92 for avian cells and 0.84 for human cells, and virulence genetic factors including H5N1 hemagglutinin genes, neuraminidase with enhanced human receptor binding mutations, and polymerase genes with mammalian adaptation markers scoring 0.91 for virulence potential, using weighted fusion algorithms that assign 35% importance to receptor binding characteristics, 30% to adherence capability, and 35% to virulence factors based on historical pandemic analysis, ultimately generating pandemic threat scores of 0.87 for human transmission potential, 0.82 for disease severity risk, and 0.89 for overall pandemic threat level, thereby providing quantitative risk assessment indicating this novel influenza strain poses high pandemic risk requiring immediate public health attention and containment measures.

For assigning the one or more pandemic threat scores to the potential zoonotic pathogens, using the AI model, the data analyzing subsystem 210 is configured to integrate the receptor binding characteristics, the host cell adherence capability, and the one or more virulence genetic factors using weighted algorithmic models within the AI model to compute composite risk metrics for each of the potential zoonotic pathogens based on the obtained analyte data. The weighted algorithmic models are computational frameworks that assign different importance values or coefficients to various input factors based on their relative significance in determining outcomes, using mathematical algorithms that multiply each factor by its assigned weight before combining them into final calculations. The composite risk metrics are unified numerical measures that result from combining multiple individual risk factors into single comprehensive scores, representing the overall risk level by integrating various contributing elements rather than considering them separately.

The process of integrating the receptor binding characteristics, the host cell adherence capability, and the one or more virulence genetic factors using weighted algorithmic models within the AI model to compute composite risk metrics for each of the potential zoonotic pathogens based on the obtained analyte data, is implemented through Multi-Modal Fusion Neural Networks that systematically combine heterogeneous molecular evidence sources through learned weight optimization and feature integration algorithms. The network employs Attention-Weighted Integration Layers that process receptor binding characteristics including binding affinity scores, structural compatibility measures, and cellular entry potential alongside host cell adherence capability encompassing adhesion protein concentrations, attachment mechanism indicators, and cellular binding efficiency, together with virulence genetic factors including toxin-encoding genes, immune evasion markers, and pathogenicity indicators, using self-attention mechanisms that automatically learn optimal weighted algorithmic models by assigning importance coefficients to each molecular evidence type based on their predictive power for pandemic risk assessment. The Multi-Modal Fusion Neural Network applies Composite Scoring Algorithms that process the weighted molecular evidence through non-linear transformation functions, normalization procedures that ensure different measurement scales are appropriately combined, and ensemble integration methods that generate unified composite risk metrics representing the overall pandemic threat level for each pathogen by mathematically combining all molecular evidence sources according to their learned importance weights.

For example, when analyzing a novel bat coronavirus with pandemic potential, the Multi-Modal Fusion Neural Network integrates previously extracted molecular evidence to compute composite risk metrics by processing receptor binding characteristics showing ACE2 binding affinity of 0.89, structural compatibility score of 0.82, and cellular entry potential of 0.91, host cell adherence capability demonstrating spike protein adhesion strength of 0.87, membrane fusion efficiency of 0.84, and overall attachment capability of 0.88, and virulence genetic factors including furin cleavage site presence scoring 0.93, immune evasion gene markers at 0.86, and pathogenicity island completeness at 0.90, using weighted algorithmic models that assign 40% weight to receptor binding characteristics (0.89×0.4=0.356), 35% weight to adherence capability (0.88×0.35=0.308), and 25% weight to virulence factors (0.90×0.25=0.225) based on learned importance from historical pandemic data, ultimately computing composite risk metrics of 0.889 (0.356+0.308+0.225) for overall pandemic threat, 0.867 for human transmission potential, and 0.891 for disease severity risk, thereby providing unified numerical assessments that integrate all molecular evidence sources into comprehensive risk scores indicating this coronavirus poses extremely high pandemic threat requiring immediate global health surveillance and preparedness measures.

For assigning the one or more pandemic threat scores to the potential zoonotic pathogens, using the AI model, the data analyzing subsystem 210 is further configured to assign the one or more pandemic threat scores to each of the potential zoonotic pathogens by applying multi-factor risk assessment algorithms within the AI model that combine composite risk metrics derived from the receptor binding characteristics, the host cell adherence capability, and the one or more virulence genetic factors, identified in the obtained analyte data. The multi-factor risk assessment algorithms are computational methods that simultaneously evaluate and process multiple different risk factors or variables to generate comprehensive risk evaluations, using mathematical models that consider the interactions and combined effects of various contributing elements rather than analyzing single factors in isolation. The composite risk metrics indicates that the numerical risk measures have been calculated and obtained through systematic analysis and integration of the specified molecular evidence sources, representing processed and quantified risk information extracted from the original molecular data.

The process of assigning the one or more pandemic threat scores to each of the potential zoonotic pathogens by applying multi-factor risk assessment algorithms within the AI model that combine composite risk metrics derived from the receptor binding characteristics, the host cell adherence capability, and the one or more virulence genetic factors, identified in the obtained analyte data, is implemented through Bayesian Multi-Factor Risk Assessment Neural Networks that systematically evaluate multiple risk dimensions simultaneously to generate final pandemic threat scores for individual pathogens. The network employs Multi-Factor Integration Algorithms that process previously computed composite risk metrics derived from receptor binding characteristics including cellular entry potential scores, binding affinity measurements, and structural compatibility indices, along with composite risk metrics derived from host cell adherence capability encompassing attachment strength scores, adhesion protein effectiveness, and cellular binding efficiency measures, together with composite risk metrics derived from virulence genetic factors including toxin production potential, immune evasion capabilities, and pathogenicity indicators, using multi-factor risk assessment algorithms that apply Bayesian inference methods to combine these diverse risk metrics while accounting for uncertainty, interdependencies between risk factors, and probabilistic relationships that influence overall pandemic threat levels. The Bayesian Multi-Factor Risk Assessment Neural Network utilizes Score Assignment Modules that process the combined risk evidence through probabilistic decision frameworks, uncertainty quantification algorithms, and confidence interval calculations to assign pandemic threat scores ranging from 0 to 1 for each pathogen, where scores represent the probability of pandemic potential based on comprehensive multi-factor risk analysis.

For example, when evaluating three potential zoonotic pathogens from wildlife surveillance, the Bayesian Multi-Factor Risk Assessment Neural Network assigns pandemic threat scores by applying multi-factor risk assessment algorithms that combine composite risk metrics where Pathogen A (novel H5N1 influenza) receives composite risk metrics derived from receptor binding characteristics of 0.89 for human receptor binding, derived from host cell adherence capability of 0.84 for respiratory epithelium attachment, and derived from virulence genetic factors of 0.92 for high pathogenicity markers, which the multi-factor algorithms combine using Bayesian integration to assign a final pandemic threat score of 0.88, while Pathogen B (bat coronavirus) receives composite metrics of 0.76, 0.82, and 0.79 respectively that combine to produce an assigned pandemic threat score of 0.79, and Pathogen C (swine influenza variant) receives composite metrics of 0.67, 0.71, and 0.68 that combine to generate an assigned pandemic threat score of 0.69, thereby providing systematic assignment of pandemic threat scores based on comprehensive multi-factor risk assessment that considers all molecular evidence sources identified in the obtained analyte data to rank H5N1 influenza as the highest pandemic threat requiring immediate containment measures.

For analyzing the obtained analyte data comprising the one or more analytes, to determine the biological status information, the data analyzing subsystem 210 is further configured to determine the biological status information indicative of the response of the one or more resources to each of the one or more pathogens comprising the potential zoonotic pathogens, based on the one or more pandemic threat scores assigned to the potential zoonotic pathogens, using the AI model. The biological status information refers to comprehensive data describing the physiological, immunological, and pathological condition of living organisms, including immune system activation levels, disease progression indicators, infection severity markers, cellular responses, metabolic changes, and overall health status assessments. The response refers to the biological reactions, physiological changes, immune system activations, and adaptive mechanisms that occur in living organisms when exposed to or infected by pathogens. The one or more resources are the biological entities being analyzed in the system, specifically including environments (air, soil, water), humans (patients, populations, individuals), and animals (livestock, wildlife, pets) that serve as hosts or potential hosts for pathogen infection. The one or more pathogens refers to the disease-causing microorganisms (viruses, bacteria, fungi, parasites) that have been identified and analyzed in the obtained analyte data. The potential zoonotic pathogens are the subset of identified pathogens that have been previously classified as having the capability or likelihood to transmit from animals to humans or between different animal species, representing infectious agents that pose cross-species transmission risks.

The process of determining the biological status information is implemented through Biological Response Prediction Neural Networks that systematically translate pandemic threat scores into specific biological response assessments for different host organisms. The network employs Threat-to-Response Mapping Algorithms that process the one or more pandemic threat scores for individual potential zoonotic pathogens through learned transformation functions that correlate pathogen threat levels with expected biological responses in the one or more resources, using training data from historical infection studies, clinical outcomes, and epidemiological records to establish mathematical relationships between pathogen threat scores and host response severity. The Biological Response Prediction Neural Network applies Multi-Host Response Modeling that generates specific biological status information for each resource type by processing pandemic threat scores through resource-specific neural network branches that account for species-specific immune systems, physiological differences, and susceptibility patterns, producing tailored response predictions that reflect how humans, animals, and environmental systems differently respond to the same pathogen threat levels.

For example, when analyzing pandemic threat scores for a novel H7N9 influenza strain assigned a pandemic threat score of 0.85, the Biological Response Prediction Neural Network determines biological status information by processing this high threat score to predict severe biological responses across the one or more resources, generating biological status information indicative of responses including for human resources: severe respiratory distress with 0.82 probability, cytokine storm syndrome with 0.78 likelihood, and 15-25% mortality risk based on the 0.85 pandemic threat score, for avian resources: 90-95% mortality rate in poultry populations, rapid viral shedding with high transmission rates, and complete flock devastation within 48-72 hours, and for environmental resources: persistent viral contamination in water sources for 14-21 days, aerosol transmission capability in enclosed spaces, and requirement for extensive decontamination protocols, thereby determining comprehensive biological status information that translates the single pandemic threat score of 0.85 into specific, actionable biological response predictions that inform public health preparedness, veterinary interventions, and environmental safety measures for each of the potential zoonotic pathogens based on their assigned threat levels.

For determining the biological status information, using the AI model, the data analyzing subsystem 210 is configured to correlate the one or more pandemic threat scores assigned to the potential zoonotic pathogens with resource-specific response patterns stored in the multianalyte database. The AI model matches high pandemic threat scores to corresponding biological response indicators for each of the one or more resources. The resource-specific response patterns are characteristic biological reaction profiles, physiological response signatures, and health outcome patterns that are unique to particular types of organisms or environments, including species-specific immune responses, tissue-specific pathological changes, and environment-specific contamination patterns stored as reference data.

The process of correlating the one or more pandemic threat scores assigned to the potential zoonotic pathogens with resource-specific response patterns stored in the multianalyte database, is implemented through Similarity-Based Correlation Neural Networks that systematically establish relationships between current pathogen threat levels and historical biological response data through multi-stage correlation analysis. The Similarity-Based Correlation Neural Networks first employs Threat Score Preprocessing Modules that normalize and standardize the one or more pandemic threat scores assigned to the potential zoonotic pathogens by converting raw threat values into standardized formats compatible with database query systems, applying scaling algorithms that ensure threat scores can be effectively compared against historical threat levels from previous pathogen outbreaks stored in the reference database. The data analyzing subsystem 210 then utilizes Database Query Networks that systematically search through resource-specific response patterns stored in the multianalyte database by executing similarity-based queries that identify historical pathogen cases with comparable threat scores, using indexing algorithms and pattern matching functions that retrieve relevant biological response data from previous infections, clinical studies, and epidemiological records organized by resource type (human, animal, environmental). The network subsequently applies Pattern Matching Algorithms where the AI model matches high pandemic threat scores by comparing current threat values against threshold criteria that define high-risk categories, using learned decision boundaries and classification rules that identify when current threat scores fall within ranges associated with severe biological outcomes, enabling the system to focus correlation analysis on the most relevant high-threat historical cases. Finally, the system implements Response Indicator Mapping Networks that establish connections between matched high-threat cases and corresponding biological response indicators for each of the one or more resources by processing historical response data through resource-specific neural network branches that generate tailored biological response predictions, using learned associations between threat levels and specific biological outcomes such as infection rates, mortality patterns, immune response profiles, and environmental contamination levels that are unique to each resource type.

For example, when analyzing a novel H5N1 influenza strain with pandemic threat scores of 0.91 for human transmission potential, 0.87 for avian infection capability, and 0.79 for environmental persistence, the Similarity-Based Correlation Neural Network correlates these high pandemic threat scores with resource-specific response patterns stored in the multianalyte database by first preprocessing the threat scores and querying historical data from previous H5N1 outbreaks, 1918 Spanish flu, and other high-threat influenza pandemics, where the AI model matches the 0.91 human threat score against stored patterns from the 1918 pandemic (threat score 0.89) and 2009 H1N1 pandemic (threat score 0.76), identifying corresponding biological response indicators including 25-40% infection rates, severe respiratory distress in 15-20% of cases, cytokine storm syndrome with IL-6 levels exceeding 100 pg/mL, and case fatality rates of 2-5% based on historical human response patterns, matches the 0.87 avian threat score with stored patterns from previous H5N1 poultry outbreaks showing corresponding biological response indicators including 90-100% mortality in infected flocks, viral shedding periods of 10-14 days, and rapid transmission rates of 0.8-1.2 secondary infections per infected bird, and matches the 0.79 environmental threat score with stored patterns indicating corresponding biological response indicators including 21-28 day survival on surfaces, aerosol transmission capability up to 6 feet, and requirement for quaternary ammonium disinfection protocols, thereby establishing comprehensive correlations between current high pandemic threat assessments and expected severe biological outcomes for each of the one or more resources based on historical response patterns from similar high-threat influenza strains stored in the multianalyte database.

For determining the biological status information, using the AI model, the data analyzing subsystem 210 is further configured to analyze resource-specific vulnerability factors using the AI model by processing the obtained analyte data to identify immune system markers, stress response proteins, and inflammatory indicators specific to each of the one or more resources in response to the potential zoonotic pathogens with assigned pandemic threat scores. The resource-specific vulnerability factors are biological characteristics, genetic predispositions, physiological weaknesses, and susceptibility markers that are unique to particular types of organisms or environments, determining how severely each resource type may be affected by pathogen exposure. The immune system markers are specific molecules, proteins, cells, or genetic expressions that indicate the activation state, functionality, and response capacity of the immune system, including antibodies, cytokines, immune cell counts, and immunological pathway indicators. The stress response proteins are specific proteins produced by cells and organisms when exposed to harmful conditions, including heat shock proteins, oxidative stress markers, cellular damage indicators, and adaptive response molecules that indicate physiological stress levels. The inflammatory indicators are molecular markers, proteins, or cellular signals that demonstrate the presence and intensity of inflammatory responses, including pro-inflammatory cytokines, acute phase proteins, inflammatory pathway activators, and tissue inflammation markers.

The process of analyzing resource-specific vulnerability factors, is implemented through Multi-Resource Vulnerability Analysis Neural Networks that systematically examine biological susceptibility patterns across different organism types through specialized analytical modules. The network first employs Resource Classification Networks that categorize the obtained analyte data by resource type (human, animal, environmental) using learned feature patterns that distinguish molecular signatures from different biological sources, applying taxonomic classification algorithms and species-specific molecular markers to ensure subsequent vulnerability analysis is tailored to the appropriate biological context. The system then utilizes Pathogen-Response Correlation Modules that link the potential zoonotic pathogens with assigned pandemic threat scores to expected biological responses by processing threat score information through learned associations between pathogen characteristics and host vulnerability patterns, using historical infection data and epidemiological records to establish which molecular markers should be prioritized for each pathogen-resource combination. The network subsequently implements Multi-Marker Identification Networks that simultaneously identify immune system markers, stress response proteins, and inflammatory indicators through parallel processing pathways where specialized sub-networks focus on each marker type, using Immune Marker Detection Networks that recognize antibody levels, cytokine concentrations, and immune cell activation patterns, Stress Protein Recognition Networks that identify heat shock proteins, oxidative stress markers, and cellular damage indicators, and Inflammatory Indicator Classification Networks that detect pro-inflammatory cytokines, acute phase proteins, and tissue inflammation signals. Finally, the system applies Resource-Specific Adaptation Algorithms that customize the identification process to be specific to each of the one or more resources by applying learned biological differences between humans, animals, and environmental systems, using species-specific reference ranges, organ-specific expression patterns, and resource-tailored analytical thresholds that account for the unique physiological and immunological characteristics of each resource type.

For example, when analyzing samples from a COVID-19 outbreak involving humans, domestic cats, and environmental surfaces exposed to SARS-CoV-2 with assigned pandemic threat scores of 0.89 for human transmission, 0.67 for feline infection, and 0.72 for environmental persistence, the Multi-Resource Vulnerability Analysis Neural Network analyzes resource-specific vulnerability factors by processing the obtained analyte data to identify distinct molecular markers specific to each resource type, where for human samples the system identifies immune system markers including elevated IL-6 levels at 89 pg/mL indicating cytokine storm, decreased CD4+ T-cell counts at 450 cells/μL showing immune suppression, and increased neutralizing antibody titers at 1:640 demonstrating adaptive immune response, identifies stress response proteins including heat shock protein 70 at 156 ng/mL indicating cellular stress, elevated cortisol at 28 μg/dL showing systemic stress response, and increased oxidative stress markers with malondialdehyde at 3.2 μmol/L, and identifies inflammatory indicators including C-reactive protein at 45 mg/L, tumor necrosis factor-α at 67 pg/mL, and interleukin-1β at 23 pg/mL indicating severe inflammatory response, while for feline samples the system identifies species-specific immune system markers including feline interferon-γ at 34 pg/mL, reduced feline CD8+ cells, and elevated feline immunoglobulin G, identifies stress response proteins including feline-specific heat shock proteins and elevated feline cortisol levels, and identifies inflammatory indicators including feline-specific acute phase proteins and species-adapted cytokine profiles, and for environmental samples identifies contamination-related stress indicators including viral RNA persistence markers, surface protein degradation products, and environmental stability factors, thereby providing comprehensive resource-specific vulnerability analysis that reveals how the same pathogen with high pandemic threat scores affects different biological systems through distinct molecular pathways and vulnerability patterns.

For determining the biological status information, using the AI model, the data analyzing subsystem 210 is further configured to generate pathogen-specific biological impact assessments using the AI model, by combining the one or more pandemic threat scores with resource response data from the obtained analyte data to determine infection severity, progression rates, and recovery potential for each of the potential zoonotic pathogens affecting each of the one or more resources. The pathogen-specific biological impact assessments are comprehensive evaluations that quantify the specific biological effects, health consequences, and physiological impacts that individual disease-causing organisms have on host organisms, including detailed analyses of infection outcomes, disease progression patterns, and recovery trajectories unique to each pathogen. The resource response data are biological measurements, physiological parameters, and molecular markers that indicate how organisms react to pathogen exposure, including immune responses, cellular changes, metabolic alterations, and other measurable biological reactions. The infection severity refers to the intensity, magnitude, or degree of disease impact on host organisms, typically measured through clinical parameters, molecular markers, and physiological indicators that quantify how seriously the infection affects normal biological functions. The progression rates are quantitative measures of how quickly diseases advance, spread, or worsen over time, including temporal patterns of symptom development, viral load changes, immune response evolution, and pathological progression speeds. The recovery potential represents the likelihood, probability, or capacity for organisms to return to normal health status following infection, including healing rates, immune clearance efficiency, and restoration of normal physiological functions.

The process of generating pathogen-specific biological impact assessments is implemented through Integrated Impact Assessment Neural Networks that systematically merge threat level information with biological response measurements to produce comprehensive pathogen-specific impact evaluations. The network first employs Data Fusion Preprocessing Modules that combine the one or more pandemic threat scores with resource response data from the obtained analyte data by normalizing threat scores and biological response measurements into compatible numerical formats, using feature scaling algorithms and data alignment procedures that ensure pandemic threat information can be mathematically integrated with molecular response data including immune markers, stress proteins, and inflammatory indicators previously identified from the analyte samples. The system then utilizes Pathogen-Specific Analysis Networks that process the combined data through specialized analytical pathways designed for each of the potential zoonotic pathogens, using learned pathogen characteristics and historical infection patterns to customize impact assessment algorithms for individual disease-causing organisms, applying pathogen-specific neural network branches that account for unique virulence factors, transmission mechanisms, and biological effects associated with each identified zoonotic pathogen. The network subsequently implements Multi-Parameter Assessment Algorithms that systematically determine infection severity, progression rates, and recovery potential through parallel processing modules where Severity Assessment Networks analyze combined threat scores and response data to quantify disease intensity using clinical severity scales and molecular severity markers, Progression Rate Calculation Networks process temporal patterns in biological response data to determine disease advancement speeds and symptom development timelines, and Recovery Potential Prediction Networks evaluate immune response strength, cellular repair markers, and physiological resilience indicators to estimate healing likelihood and recovery timeframes. Finally, the system applies Resource-Specific Impact Modeling that generates tailored assessments for each of the one or more resources by processing the integrated data through resource-specific neural network branches that account for biological differences between humans, animals, and environmental systems, using species-specific physiological parameters, immune system characteristics, and susceptibility patterns to produce customized impact assessments that reflect how the same pathogen affects different biological entities through distinct pathological mechanisms and recovery pathways.

For example, when analyzing a novel H7N9 influenza strain with pandemic threat scores of 0.87 for human transmission and 0.82 for avian infection, the Integrated Impact Assessment Neural Network generates pathogen-specific biological impact assessments by combining these threat scores with resource response data from the obtained analyte data including human samples showing elevated IL-6 at 95 pg/mL, decreased lymphocyte counts at 800 cells/μL, and increased viral load at 10\hat{ }6 copies/mL, along with avian samples showing severe respiratory distress markers, elevated avian interferon levels, and high viral shedding rates, to determine comprehensive impact parameters where for each of the potential zoonotic pathogens affecting human resources the system calculates infection severity scores of 0.89 indicating severe respiratory illness with high hospitalization risk, progression rates of 3.2 days for symptom onset and 7-10 days for peak severity based on viral load kinetics and immune response patterns, and recovery potential of 0.67 indicating moderate recovery likelihood with 21-28 day recovery timeframes based on lymphocyte recovery patterns and inflammatory resolution markers, while for avian resources affecting each of the one or more resources the assessment determines infection severity of 0.94 indicating near-fatal disease with 85-95% mortality risk, progression rates of 1.5 days for symptom onset and 3-5 days for fatal outcomes based on rapid viral replication and immune system collapse patterns, and recovery potential of 0.23 indicating poor survival prospects with only 5-15% recovery rates based on severe immunosuppression and multi-organ failure indicators, thereby providing comprehensive pathogen-specific biological impact assessments that integrate pandemic threat levels with actual biological response evidence to generate actionable clinical and veterinary guidance for managing H7N9 influenza infections across different host species.

For determining the biological status information, using the AI model, the data analyzing subsystem 210 is further configured to determine resource response severity levels using the AI model by weighting the one or more pandemic threat scores against resource-specific susceptibility indicators identified in the obtained analyte data, wherein higher pandemic threat scores correlate to severe biological status information for vulnerable resources. The resource response severity levels are quantitative measures that indicate the intensity, magnitude, or degree of biological impact that pathogens have on different types of organisms or environments, representing how severely each resource type is affected by pathogen exposure on a graduated scale. The resource-specific susceptibility indicators are biological markers, genetic factors, physiological characteristics, or molecular signatures that reveal how vulnerable particular types of organisms or environments are to pathogen infection, including immune system weaknesses, genetic predispositions, and biological risk factors unique to each resource type. The severe biological status information refers to intense, serious, or critical biological conditions, health outcomes, or physiological states that indicate significant disease impact, including life-threatening symptoms, major organ dysfunction, or severe pathological changes. The higher pandemic threat scores correlate to the severe biological status information for the vulnerable resources indicates that the correlation applies specifically to organisms or environments that have been identified as having high susceptibility or weakness to pathogen infection based on their biological characteristics.

The process of determining resource response severity levels is implemented through Weighted Severity Assessment Neural Networks that systematically calculate biological impact intensity by mathematically balancing pathogen threat levels with host vulnerability characteristics. The network first employs Susceptibility Indicator Extraction Modules that systematically scan the obtained analyte data to identify resource-specific susceptibility indicators including genetic vulnerability markers such as ACE2 receptor expression levels, immune system deficiency indicators like reduced immunoglobulin concentrations, physiological weakness markers including advanced age proteins or chronic disease indicators,

and species-specific susceptibility factors such as receptor binding compatibility scores, using pattern recognition algorithms trained on extensive databases of vulnerability factors to detect molecular signatures that indicate increased infection risk for each resource type. The system then utilizes Threat Score Processing Networks that normalize and prepare the one or more pandemic threat scores for mathematical integration with susceptibility data, applying scaling algorithms that ensure threat scores can be effectively combined with vulnerability indicators through weighted mathematical operations. The network subsequently implements Weighted Integration Algorithms that perform the core weighting process by mathematically combining pandemic threat scores with susceptibility indicators using learned weight matrices that reflect the relative importance of threat level versus vulnerability factors in determining overall severity, where the weighting operation multiplies threat scores by vulnerability-adjusted coefficients that increase the final severity calculation when high threat scores are combined with high susceptibility indicators, creating the mathematical relationship wherein higher pandemic threat scores correlate to severe biological status information for vulnerable resources. Finally, the system applies Severity Level Classification Networks that process the weighted integration results through graduated severity scales to determine resource response severity levels, using threshold-based classification algorithms that categorize the weighted scores into discrete severity categories such as mild (0.0-0.3), moderate (0.3-0.6), severe (0.6-0.8), and critical (0.8-1.0), with additional neural network layers that generate confidence intervals and uncertainty estimates for each severity determination.

For example, when analyzing COVID-19 samples from elderly patients with comorbidities, the Weighted Severity Assessment Neural Network determines resource response severity levels by first identifying resource-specific susceptibility indicators in the obtained analyte data including elevated ACE2 receptor expression at 2.3-fold normal levels indicating increased viral entry potential, reduced CD4+ T-cell counts at 350 cells/μL showing immune system compromise, elevated inflammatory markers including IL-6 at 78 pg/mL indicating cytokine storm susceptibility, and chronic disease markers including elevated HbA1c at 8.2% indicating diabetes-related vulnerability, then weighting the SARS-CoV-2 pandemic threat score of 0.85 against these high susceptibility indicators by applying learned weight coefficients where the threat score is multiplied by a vulnerability amplification factor of 1.4 based on the combination of age-related immune decline (weight 0.3), diabetes susceptibility (weight 0.4), and elevated ACE2 expression (weight 0.3), resulting in a weighted severity calculation of 0.85×1.4=1.19 which is normalized to 0.95 on the severity scale, thereby determining a resource response severity level of “Critical” (0.95) that reflects how the higher pandemic threat score correlates to severe biological status information for vulnerable resources, predicting severe COVID-19 outcomes including 85% hospitalization probability, 45% ICU admission likelihood, and 25% mortality risk based on the mathematical integration of high pathogen threat with multiple vulnerability factors identified in the molecular analysis.

For determining the biological status information, using the AI model, the data analyzing subsystem 210 is further configured to determine the biological status information indicative of the response of the one or more resources to each of the one or more pathogens comprising the potential zoonotic pathogens by integrating the pathogen-specific biological impact assessments and the resource response severity levels through the AI model to generate comprehensive biological status information that reflect the pandemic threat level of each potential zoonotic pathogen on each of the one or more resources. The resource response severity levels are quantitative measures that indicate the intensity, magnitude, or degree of biological impact that pathogens have on different types of organisms or environments, representing how severely each resource type is affected by pathogen exposure on a graduated scale. The pandemic threat level refers to the degree of risk or potential danger that each pathogen poses for causing widespread disease outbreaks across multiple populations, countries, or continents.

The process of determining the biological status information is implemented through Comprehensive Integration Neural Networks that systematically merge multiple analytical results into unified biological status assessments. The network first employs Data Integration Preprocessing Modules that prepare the pathogen-specific biological impact assessments (containing infection severity scores, progression rates, and recovery potential) and the resource response severity levels (containing weighted vulnerability assessments) for mathematical combination by normalizing different measurement scales, aligning temporal parameters, and ensuring data compatibility across different analytical outputs. The system then utilizes Multi-Dimensional Fusion Algorithms that perform the core integrating process through the AI model by mathematically combining impact assessments and severity levels using learned integration weights that reflect the relative importance of biological impact versus severity factors, applying tensor fusion operations that preserve the individual contributions of each analytical component while creating unified representations that capture the complete biological response picture. The network subsequently implements Pathogen-Resource Mapping Networks that process the integrated data to generate comprehensive biological status information for each potential zoonotic pathogen and each of the one or more resources through specialized neural network branches that create pathogen-resource specific assessments, using learned associations between integrated biological evidence and expected health outcomes to produce detailed status reports that include infection likelihood, disease severity predictions, recovery timelines, and intervention requirements. Finally, the data analyzing subsystem 210 applies Threat Level Reflection Algorithms that ensure the generated biological status information accurately reflects the pandemic threat level by incorporating pandemic threat scores into the final biological assessments, using mathematical scaling functions that adjust biological status predictions based on pathogen threat levels so that higher threat pathogens generate more severe biological status predictions and lower threat pathogens produce milder status assessments, creating direct correspondence between pandemic risk and predicted biological outcomes.

For example, when analyzing a novel H5N1 influenza strain with pandemic threat score of 0.89 affecting human and avian resources, the Comprehensive Integration Neural Network determines comprehensive biological status information by integrating previously calculated pathogen-specific biological impact assessments showing human infection severity of 0.87, progression rate of 4.2 days to peak symptoms, and recovery potential of 0.64, along with avian impact assessments showing infection severity of 0.94, progression rate of 2.1 days to mortality, and recovery potential of 0.18, and resource response severity levels of 0.82 for humans (based on respiratory vulnerability and immune factors) and 0.96 for avians (based on species-specific H5N1 susceptibility), through the AI model using integration algorithms that combine impact scores (0.87×0.3+4.2×0.2+0.64×0.2=1.23) with severity levels (0.82×0.3=0.25) to produce integrated human assessment score of 1.48, and avian integration (0.94×0.3+2.1×0.2+0.18×0.2=0.74) with severity (0.96×0.3=0.29) producing avian score of 1.03, then generating comprehensive biological status information that reflects the pandemic threat level of 0.89 by scaling the integrated assessments to produce final biological status showing for humans: 89% probability of severe respiratory illness, 15-day average recovery time, 25% hospitalization rate, and requirement for antiviral intervention within 48 hours, and for avians: 95% mortality rate, 2-3 day survival time, complete flock elimination risk, and immediate culling recommendations, thereby providing comprehensive biological status information indicative of the response of the one or more resources to each of the potential zoonotic pathogens that directly corresponds to the high pandemic threat level and enables targeted medical and veterinary response planning.

In an exemplary embodiment, the plurality of subsystems 118 further includes the output subsystem 212 that is communicatively connected to the one or more hardware processors 114. The output subsystem 212 is configured to provide the biological status information indicative of the response of the one or more resources to each of the one or more pathogens comprising the potential zoonotic pathogens, as the output to the display device (e.g., user device 106) associated with the one or more users.

The output subsystem 212 is a comprehensive computational framework that serves as the final interface between the AI-based system and end users, functioning as a sophisticated data presentation and delivery mechanism that transforms complex analytical results into actionable information. This output subsystem 212 operates through multiple integrated components including data formatting engines that convert raw analytical results from previous AI processing stages into structured, readable formats by applying natural language generation algorithms to translate technical biological assessments into comprehensible reports, statistical summaries, and clinical recommendations, while simultaneously creating visual representations through automated chart generation, graph plotting, and dashboard creation that present biological status information in intuitive graphical formats. The output subsystem 212 incorporates user interface management systems that customize information presentation based on user roles and requirements, where healthcare professionals receive clinical decision support interfaces with patient-specific risk assessments and treatment recommendations, researchers obtain detailed molecular data visualizations with statistical analyses and predictive modeling results, public health officials get epidemiological dashboards with population-level risk maps and outbreak tracking information, and veterinarians receive species-specific health status reports with intervention guidance and biosecurity recommendations. The output subsystem 212 includes multi-device compatibility frameworks that ensure seamless information delivery across various display technologies by implementing responsive design principles that automatically adjust content layout, font sizes, and graphical elements based on screen dimensions and device capabilities, supporting everything from large wall-mounted displays in emergency operations centers to mobile devices used by field personnel. The output subsystem 212 features real-time data streaming capabilities that provide continuous updates of biological status information as new analytical results become available, using secure communication protocols and data synchronization mechanisms that ensure users receive the most current pathogen surveillance information with minimal latency. Additionally, the output subsystem 212 incorporates security and access control mechanisms that protect sensitive biological and health information through user authentication, role-based access permissions, and encrypted data transmission, while maintaining audit trails that track information access and usage for compliance and security purposes. The output subsystem 212 also includes alert and notification systems that automatically generate and distribute urgent warnings when biological status information indicates high-risk situations, using configurable threshold-based triggers that send immediate notifications through multiple channels including email, SMS, mobile app notifications, and system alerts to ensure critical information reaches appropriate users without delay.

For example, when analyzing a novel coronavirus outbreak affecting multiple species, the output subsystem 212 provides the biological status information by processing comprehensive analytical results showing human infection severity of 0.84 with cytokine storm risk, bat reservoir confirmation with 0.91 zoonotic transmission potential, and environmental surface persistence of 14-21 days, then configuring specialized delivery mechanisms where hospital emergency departments receive real-time clinical dashboards displayed on large monitors showing patient triage categories with color-coded risk levels (red for severe cases requiring immediate ICU admission, yellow for moderate cases needing hospitalization, green for mild cases suitable for outpatient monitoring), wildlife surveillance teams get mobile tablet interfaces showing GPS-mapped bat colony infection status with sampling recommendations and biosafety protocols, environmental health inspectors receive smartphone notifications with contaminated location alerts and decontamination requirements, and public health command centers obtain comprehensive wall-display dashboards showing regional outbreak progression maps, resource allocation recommendations, and intervention effectiveness metrics, thereby providing biological status information indicative of the response of the one or more resources (humans showing severe respiratory symptoms and immune dysregulation, bats demonstrating asymptomatic viral shedding and reservoir maintenance, environments exhibiting prolonged contamination and transmission risk) to each of the one or more pathogens comprising the potential zoonotic pathogens as an output to display devices associated with one or more users who can immediately access role-specific, actionable information formatted for their professional needs and decision-making requirements, enabling coordinated outbreak response across medical, veterinary, environmental, and public health sectors through synchronized information delivery that ensures all stakeholders have access to current, relevant biological status information for effective pandemic prevention and control measures.

In an exemplary embodiment, the plurality of subsystems 118 further includes the updating subsystem 214 that is communicatively connected to the one or more hardware processors 114. The updating subsystem 214 is configured to update the multianalyte database 104 based on at least one of: a unique analyte and a combination of one or more unique analytes in the analyte data, stored in the multianalyte database 104. The unique analyte refers to a previously unencountered or novel molecular marker, biological compound, or analytical measurement that has not been previously stored or characterized in the existing database. The updating subsystem 214 is a sophisticated adaptive learning framework that continuously enhances the multianalyte database through intelligent data integration and pattern recognition mechanisms, functioning as a dynamic knowledge management system that ensures the pathogen surveillance system remains current with emerging biological threats and novel molecular signatures. This updating subsystem 214 operates through multiple integrated components including Novel Analyte Detection Engines that continuously scan incoming analytical data to identify unique analytes that have not been previously encountered in the database, using similarity comparison algorithms, molecular fingerprinting techniques, and statistical novelty detection methods to distinguish new molecular markers from existing database entries, while applying confidence scoring mechanisms that validate the uniqueness and significance of detected novel analytes before triggering database updates. The updating subsystem 214 incorporates Pattern Recognition and Classification Systems that analyze combinations of one or more unique analytes by examining how novel molecular markers appear together in specific arrangements, using machine learning algorithms to identify meaningful relationships between multiple unique analytes that may represent new pathogen signatures, emerging disease patterns, or previously unknown biological pathways, while applying clustering algorithms and association rule mining to discover significant patterns that warrant database incorporation. The updating subsystem 214 includes Database Integration Mechanisms that systematically incorporate validated unique analytes and analyte combinations into the existing multianalyte database structure by creating new data entries, establishing relationships with existing molecular patterns, updating reference libraries, and modifying classification algorithms to accommodate new molecular information, while maintaining data integrity, version control, and backward compatibility with existing analytical processes. The updating subsystem 214 features Adaptive Learning Algorithms that not only add new information but also refine existing database patterns based on accumulated evidence from unique analyte discoveries, using machine learning techniques to improve pattern recognition accuracy, update pathogen identification thresholds, and enhance biological status prediction capabilities as more diverse molecular data becomes available through ongoing surveillance activities. Additionally, the updating subsystem 214 incorporates Quality Control and Validation Frameworks that ensure database updates maintain scientific accuracy and analytical reliability by implementing peer review mechanisms, cross-validation procedures, and expert system checks that verify the biological significance and diagnostic value of unique analytes before permanent database integration, while maintaining audit trails that document all database modifications for scientific transparency and regulatory compliance.

For example, when analyzing respiratory samples from a novel viral outbreak, the updating subsystem 214 updates the multianalyte database 104 by first detecting a unique analyte in the form of a previously uncharacterized viral protein with molecular weight 45.2 kDa and specific amino acid sequence patterns that do not match any existing entries in the database, triggering the novelty detection algorithms to flag this protein as a significant new molecular marker requiring database integration, then identifying a combination of one or more unique analytes including the novel viral protein appearing together with an unusual host immune response pattern involving elevated interferon-λ4 levels (not previously associated with respiratory infections) and a distinctive microRNA expression profile (miR-2087 and miR-3156) that collectively represent a new pathogen-host interaction signature, prompting the pattern recognition systems to classify this three-component molecular combination as a novel diagnostic pattern warranting database incorporation, subsequently updating the database by creating new entries for the unique viral protein with its molecular characteristics, establishing a new pathogen category for the emerging virus, adding the novel immune response pattern to host response libraries, incorporating the distinctive microRNA profile into regulatory marker databases, and creating cross-references between all three unique analytes to establish their combined diagnostic significance, while simultaneously refining existing pattern matching algorithms to recognize similar molecular combinations in future samples, thereby ensuring the multianalyte database 104 remains current with emerging biological threats and can accurately identify this new pathogen in subsequent surveillance activities through the systematic integration of unique analytes and their combinations discovered in the analyte data from ongoing pathogen monitoring efforts.

In an embodiment, the multianalyte database 104 may be used to analyze the new analyte data to derive/generate new information from the combined new analyte data and old analyte data, using the AI model. The AI model employed for analyzing the combined new analyte data and old analyte data operates through a sophisticated Temporal Data Integration and Knowledge Discovery Framework that systematically merges historical molecular information with current analytical results to generate enhanced biological insights and improved pathogen surveillance capabilities. This AI model functions through multiple integrated analytical stages beginning with Data Fusion and Temporal Alignment Networks that process the new analyte data and old analyte data by normalizing different measurement scales, aligning temporal sequences, and creating unified data representations that enable meaningful comparison between historical and current molecular information, using advanced data preprocessing algorithms that account for technological differences, measurement variations, and temporal gaps between old and new datasets. The AI model then employs Pattern Evolution Detection Algorithms that systematically compare molecular patterns in new analyte data against established patterns in old analyte data to identify evolutionary changes, emerging trends, and novel molecular signatures that may indicate pathogen mutations, host adaptation, or new biological pathways, using machine learning techniques including sequence alignment algorithms, structural comparison methods, and statistical trend analysis to detect significant changes in molecular patterns over time. The updating subsystem 214 subsequently utilizes Knowledge Enhancement Neural Networks that derive/generate new information by identifying previously unrecognized relationships between historical and current molecular data, using deep learning architectures that can discover complex non-linear associations between molecular markers across different time periods, enabling the identification of subtle biological patterns that may not be apparent when analyzing old or new data in isolation. The AI model incorporates Predictive Modeling and Extrapolation Systems that use the combined temporal dataset to generate enhanced predictive capabilities, training machine learning models on the integrated historical and current data to improve pathogen identification accuracy, biological status prediction reliability, and outbreak forecasting precision, while using ensemble learning methods that combine insights from both historical patterns and current trends to produce more robust analytical results. Additionally, the AI model features Adaptive Learning and Database Enhancement Mechanisms that continuously refine analytical algorithms based on the insights gained from combining old and new data, using reinforcement learning techniques to improve pattern recognition accuracy, update classification thresholds, and enhance diagnostic capabilities as more temporal data becomes available, ensuring that the analytical system becomes increasingly sophisticated and accurate over time through continuous learning from the expanding combined dataset.

For example, when analyzing samples from a suspected H1N1 influenza outbreak, the multianalyte database 104 is used to analyze the new analyte data containing current viral RNA sequences, host immune response markers, and environmental persistence indicators combined with old analyte data from the 2009 H1N1 pandemic, seasonal influenza surveillance from 2010-2024, and historical swine influenza datasets, using the AI model to derive/generate new information through temporal pattern analysis that reveals the current H1N1 strain has acquired novel mutations in the hemagglutinin gene (positions 190 and 225) not present in historical data, combined with new analyte data showing unusual host immune responses including elevated IL-10 levels and reduced interferon-γ production that differ significantly from old analyte data patterns of typical H1N1 infections, enabling the AI model to generate new information including the discovery that this H1N1 variant has enhanced immune evasion capabilities (derived from comparing current immune suppression patterns with historical immune activation data), increased human-to-human transmission potential (calculated by analyzing current viral load patterns against historical transmission data), and extended environmental survival (determined by comparing current persistence markers with historical stability data), while simultaneously updating pathogen classification algorithms to recognize this evolved H1N1 variant, enhancing diagnostic accuracy from 87% to 94% through improved pattern recognition trained on the combined temporal dataset, and generating predictive models that forecast this variant's pandemic potential at 0.78 based on integrated analysis of historical pandemic patterns and current molecular characteristics, thereby demonstrating how the multianalyte database enables the AI model to analyze integrated temporal data to derive new information that significantly enhances pathogen surveillance capabilities and public health preparedness through comprehensive temporal molecular analysis.

The updating subsystem 214 is further configured to dynamically adjust the one or more pre-defined patterns associated with the one or more pre-stored analytes of the pre-stored analyte data in the multianalyte database 104, based on the analyte data obtained from the one or more samples of the one or more resources over the period of time, using an adaptive sampling process. The one or more pre-defined patterns are established molecular signatures, analyte combinations, or biological marker arrangements that have been previously identified and stored in the database as reference templates for pathogen identification, disease detection, or biological status assessment, including specific combinations of nucleic acid sequences, protein concentrations, metabolite profiles, and other molecular markers that collectively indicate particular pathogens or biological conditions. Over the period of time indicates that the adjustment process considers temporal changes and trends in the data collected across multiple time points or extended surveillance periods. The adaptive sampling process is an intelligent data collection and analysis methodology that automatically adjusts sampling strategies, pattern recognition thresholds, and analytical parameters based on observed data trends, emerging patterns, and changing biological conditions, enabling the system to optimize its surveillance effectiveness and accuracy through continuous learning and adaptation.

The AI model employed for dynamically adjusting the one or more pre-defined patterns operates through a sophisticated Adaptive Pattern Evolution Framework that continuously monitors incoming molecular data and automatically modifies existing reference patterns to maintain optimal pathogen detection accuracy and biological status assessment capabilities. This AI model functions through multiple integrated components beginning with Temporal Pattern Monitoring Networks that systematically track how the analyte data obtained from the one or more samples changes over the period of time by analyzing molecular marker trends, concentration variations, and pattern frequency shifts, using time-series analysis algorithms and statistical trend detection methods to identify when current molecular data begins to deviate significantly from established pre-defined patterns stored in the database. The AI model then employs Pattern Drift Detection Algorithms that quantify the degree of change between current molecular signatures and existing reference patterns by calculating similarity scores, correlation coefficients, and statistical distances between new and historical data, using machine learning techniques including anomaly detection, distribution shift analysis, and concept drift identification to determine when pre-defined patterns require modification to maintain diagnostic accuracy and biological relevance. The system subsequently utilizes Adaptive Pattern Refinement Networks that perform the core dynamic adjustment process by modifying existing molecular patterns based on accumulated evidence from ongoing surveillance data, using adaptive sampling process methodologies that automatically adjust pattern recognition thresholds, update molecular marker weights, and refine analyte combination rules based on observed performance metrics and emerging biological trends. The AI model incorporates Multi-Objective Optimization Algorithms that balance multiple competing factors during pattern adjustment including maintaining compatibility with historical data, optimizing detection sensitivity for emerging threats, preserving specificity to avoid false positives, and ensuring computational efficiency for real-time surveillance applications, using evolutionary algorithms and reinforcement learning techniques that continuously optimize pattern parameters based on surveillance performance feedback. Additionally, the AI model features Validation and Quality Control Mechanisms that ensure dynamically adjusted patterns maintain scientific accuracy and diagnostic reliability by implementing cross-validation procedures, expert system checks, and performance monitoring that verify adjusted patterns improve rather than degrade surveillance capabilities, while maintaining audit trails that document all pattern modifications for scientific transparency and regulatory compliance.

For example, when monitoring SARS-CoV-2 variants over an 18-month surveillance period, the updating subsystem 214 dynamically adjusts pre-defined patterns by initially storing pre-defined patterns for the original Wuhan strain including spike protein receptor binding domain sequences, nucleocapsid protein markers, and host immune response signatures (IL-6 levels 15-25 pg/mL, interferon-α 8-12 pg/mL), then continuously analyzing analyte data obtained from samples showing gradual changes including Alpha variant emergence with D614G mutation, Delta variant with L452R and T478K mutations, and Omicron variant with extensive spike protein changes, while simultaneously observing evolving host immune responses with analyte data over the period of time showing increased IL-6 levels (35-45 pg/mL for Delta, 25-35 pg/mL for Omicron) and altered interferon patterns, prompting the adaptive sampling process to automatically dynamically adjust the original pre-defined patterns by expanding spike protein sequence recognition to include variant-specific mutations, updating receptor binding domain patterns to accommodate structural changes in BA.1, BA.2, and BA.5 subvariants, modifying host immune response thresholds to reflect variant-specific cytokine profiles, and refining diagnostic sensitivity parameters to maintain 95% detection accuracy across all variants, while the AI model continuously monitors surveillance performance and further adjusts patterns when new variants like XBB.1.5 emerge with additional immune escape mutations, thereby ensuring the one or more pre-defined patterns associated with the one or more pre-stored analytes remain current and effective for accurate SARS-CoV-2 detection despite continuous viral evolution, demonstrating how the adaptive sampling process enables real-time pattern optimization that maintains surveillance system effectiveness over the period of time as biological threats evolve and change.

In an exemplary embodiment, the plurality of subsystems 118 further includes the report generating subsystem 216 that is communicatively connected to the one or more hardware processors 114. The report generating subsystem 216 is configured to generate one or more actionable reports based on the analyzed analyte data with the one or more analytes. In an embodiment, the one or more actionable reports are one or more suggestions on at least one of: decisions on health of the one or more resources, creation of one or more vaccines, one or more therapeutics, and supporting of one or more research activities, and the like.

The one or more actionable reports are comprehensive, structured documents or data presentations that provide specific, implementable recommendations and guidance based on analytical results, designed to enable immediate decision-making and practical interventions by healthcare professionals, researchers, and public health officials. The decisions on health of the one or more resources refer to clinical, veterinary, or environmental health determinations that need to be made regarding the medical treatment, care management, intervention strategies, or protective measures for humans, animals, or environmental systems based on their biological status and pathogen exposure risks. The creation of one or more vaccines involves the development, design, and production of immunological preparations that stimulate immune responses to provide protection against specific pathogens, including vaccine candidate identification, antigen selection, formulation development, and manufacturing recommendations based on pathogen characteristics identified through molecular analysis. The one or more therapeutics are medical treatments, drugs, or interventions designed to treat, cure, or manage diseases caused by identified pathogens, including antiviral medications, antibiotics, immunomodulatory drugs, supportive care protocols, and other therapeutic approaches tailored to specific pathogen-host interactions. The supporting of one or more research activities involves providing scientific guidance, data recommendations, experimental design suggestions, and analytical insights that facilitate further scientific investigation, including research prioritization, funding recommendations, collaborative opportunities, and methodological guidance for advancing pathogen surveillance and disease prevention research.

The process of generating one or more actionable reports is implemented through Intelligent Report Generation Neural Networks that systematically transform complex analytical results into structured, practical guidance documents tailored to specific user needs and decision-making contexts. This report generating system 216 operates through multiple integrated components beginning with Content Analysis and Prioritization Modules that process the analyzed analyte data with the one or more analytes by identifying the most critical findings, significant biological status information, and urgent health implications that require immediate attention, using importance ranking algorithms and risk assessment criteria to prioritize report content based on threat levels, time sensitivity, and potential impact on public health or individual patient outcomes. The report generating system 216 then employs Multi-Audience Report Customization Networks that create specialized report versions tailored to different professional audiences and decision-making contexts, where clinical reports emphasize patient-specific treatment recommendations and diagnostic guidance, public health reports focus on population-level interventions and outbreak control measures, research reports highlight scientific findings and experimental opportunities, and policy reports provide regulatory guidance and resource allocation recommendations. The report generating system 216 utilizes Natural Language Generation Algorithms that convert technical analytical results into clear, comprehensible text by translating complex molecular data, statistical analyses, and biological status assessments into professional language appropriate for each audience, using domain-specific terminology, standardized medical nomenclature, and evidence-based recommendation formats that enable immediate implementation of suggested actions. The report generating system 216 incorporates Visual Data Presentation Engines that create charts, graphs, maps, and other visual elements that enhance report comprehension and decision-making effectiveness, including risk assessment matrices, temporal trend analyses, geographic distribution maps, and comparative effectiveness tables that present analytical results in intuitive visual formats. Additionally, the report generating system 216 features Action Item Extraction and Recommendation Algorithms that systematically identify specific, implementable actions based on analytical findings, using decision tree logic and expert system rules to generate concrete recommendations for immediate implementation, including treatment protocols, intervention timelines, resource requirements, and success metrics that enable users to translate analytical insights into practical actions.

For example, when analyzing a multi-pathogen outbreak involving H5N1 influenza affecting poultry farms and SARS-CoV-2 variants in the human population, the report generating subsystem 216 generates actionable reports by processing analyzed analyte data showing H5N1 with pandemic threat score of 0.89, human infection severity of 0.82, and environmental persistence of 14 days, combined with SARS-CoV-2 variant data showing immune escape markers and increased transmissibility, to produce multiple specialized reports including a Clinical Decision Support Report providing decisions on health of the one or more resources with specific recommendations for human patients including immediate isolation protocols for suspected H5N1 cases, antiviral treatment with oseltamivir within 48 hours of symptom onset, enhanced PPE requirements for healthcare workers, and modified COVID-19 treatment protocols accounting for variant-specific characteristics, a Vaccine Development Priority Report supporting creation of one or more vaccines with recommendations to accelerate H5N1 vaccine production using reverse genetics platforms, update COVID-19 vaccine formulations to address immune escape variants, prioritize bivalent vaccine development combining H5N1 and seasonal influenza components, and establish emergency vaccine stockpiling protocols, a Therapeutic Intervention Report guiding one or more therapeutics including recommendations for combination antiviral therapy using neuraminidase inhibitors and polymerase inhibitors for H5N1 treatment, monoclonal antibody therapy selection based on variant-specific binding profiles, supportive care protocols for severe respiratory illness, and drug interaction considerations for co-infected patients, and a Research Prioritization Report for supporting research activities recommending immediate studies on H5N1 human adaptation mechanisms, cross-species transmission dynamics, vaccine effectiveness against emerging variants, therapeutic resistance monitoring, and environmental surveillance optimization, thereby providing comprehensive actionable reports that enable immediate implementation of evidence-based interventions across clinical care, vaccine development, therapeutic management, and research advancement based on systematic analysis of the molecular surveillance data.

In an exemplary embodiment, the plurality of subsystems 118 optionally includes an information generating subsystem that is communicatively connected to the one or more hardware processors 114. The information generating subsystem is configured to generate unique biological status information indicative of the response of the one or more resources to each of the one or more pathogens, using at least one of: the unique analyte and the combination of one or more unique analytes in the analyte data stored in the multianalyte database 104, based on the AI model being trained on the pre-stored analyte data associated with the one or more pre-stored analytes in the one or more pre-defined patterns.

The training process for the AI model used by the information generating subsystem to generate unique biological status information operates through a sophisticated multi-stage supervised learning framework that systematically teaches the artificial intelligence system to recognize novel molecular patterns and predict unprecedented biological responses. The training process begins with historical data preprocessing and pattern extraction where the pre-stored analyte data associated with the one or more pre-stored analytes in the one or more pre-defined patterns serves as the foundational training dataset, with data scientists and bioinformatics specialists systematically organizing historical molecular information into labeled training examples that pair specific analyte combinations with known biological outcomes, pathogen identifications, and health status assessments, using data cleaning algorithms to remove noise and inconsistencies while maintaining the integrity of established molecular patterns that represent confirmed biological relationships. The information generating subsystem then employs supervised pattern recognition training where the AI model learns to identify relationships between molecular signatures and biological outcomes by processing thousands of training examples that demonstrate how specific pre-defined patterns of pre-stored analytes correspond to particular pathogen infections, immune responses, and disease progressions, using deep learning architectures including convolutional neural networks for sequence analysis, recurrent neural networks for temporal pattern recognition, and transformer networks for complex molecular relationship modeling that enable the information generating subsystem to learn intricate associations between molecular evidence and biological status information. The training process incorporates transfer learning and domain adaptation techniques that enable the trained AI model to apply learned knowledge from pre-stored analyte data to recognize and interpret unique analytes and combinations of unique analytes that were not present in the original training dataset, using feature extraction methods that identify common molecular characteristics and biological principles that can be generalized from known patterns to novel molecular signatures, enabling the information generating subsystem to generate meaningful biological insights even when encountering previously unseen molecular combinations.

The training framework includes validation and performance optimization where the AI model's ability to generate accurate biological status information is continuously tested using held-out validation datasets and cross-validation procedures that ensure the trained system can reliably extrapolate from known molecular patterns to predict biological responses for novel analyte combinations, using performance metrics including prediction accuracy, sensitivity, specificity, and biological relevance scores that guide iterative model refinement and hyperparameter optimization. Additionally, the training process features continuous learning and model updates where the AI model is periodically retrained on expanded datasets that include newly discovered unique analytes and their associated biological outcomes, using online learning algorithms and incremental training methods that allow the model to continuously improve its ability to generate accurate biological status information as more diverse molecular data becomes available through ongoing surveillance activities.

For example, when analyzing samples from a novel zoonotic outbreak in Southeast Asia, the information generating subsystem employs a Deep Learning Ensemble AI Model comprising Convolutional Neural Networks (CNNs) for molecular sequence analysis, Recurrent Neural Networks (RNNs) for temporal pattern recognition, and Graph Neural Networks (GNNs) for molecular interaction modeling that has been trained on pre-stored analyte data including 75,000 historical pathogen samples containing pre-defined patterns such as influenza hemagglutinin sequences, coronavirus spike proteins, bacterial toxin genes, and corresponding host immune responses (cytokine profiles, antibody levels, cellular markers) from previous outbreaks including H1N1 2009, SARS-CoV-2, MERS-CoV, and avian influenza H5N1, enabling the trained AI model to generate unique biological status information when processing current samples containing unique analytes including a previously uncharacterized bat paramyxovirus with novel fusion protein sequences showing 67% similarity to Nipah virus but containing 15 unique amino acid substitutions not present in any training data, combined with the combination of one or more unique analytes including unusual host immune markers such as elevated galectin-9 levels (45 ng/mL vs normal 8-12 ng/mL), novel inflammatory protein signatures (CXCL-16 at 89 pg/mL), and distinctive microRNA expression patterns (miR-8934 and miR-7621 upregulated 4.2-fold) that collectively represent a molecular signature never documented in the multianalyte database 104, where the AI model applies learned principles from pre-stored analyte data through the CNN component analyzing the novel fusion protein structure against trained viral protein patterns to predict enhanced cell membrane binding capability with 0.89 confidence, the RNN component processing the temporal immune response patterns to forecast severe neurological complications within 7-10 days based on similarity to trained Nipah virus progression patterns, and the GNN component modeling molecular interactions between the unique viral proteins and host immune markers to predict immune evasion mechanisms and tissue tropism, ultimately generating unique biological status information indicative of the response of the one or more resources including predictions that human exposure will result in severe encephalitis with 0.84 probability, bat reservoir hosts will maintain asymptomatic infection with high viral shedding, and environmental persistence will enable aerosol transmission for 6-8 hours, while simultaneously generating novel therapeutic recommendations including ribavirin combination therapy based on structural similarity patterns learned from training data, thereby demonstrating how the trained AI model enables the information generating subsystem to produce unprecedented biological insights for completely novel pathogen-host interactions by leveraging learned molecular principles from historical surveillance data.

In an exemplary embodiment, the plurality of subsystems 118 further includes the system integrating subsystem 218 that is communicatively connected to the one or more hardware processors 114. The system integrating subsystem 218 is configured to integrate the AI-based system 102 with one or more external surveillance systems to exchange data to determine the biological status information indicative of the response of the one or more resources to each of the one or more pathogens. For example, the AI-based system 102 may detect an emerging pandemic threat and issue a warning to designated public health authorities (i.e., the external surveillance system) with a genetic code associated with the risk. Further, the AI-based system 102 driven by artificially intelligent search of the internet, may receive information that can be used in the analysis of the analyte data.

The one or more external surveillance systems are independent monitoring, detection, or data collection platforms operated by other organizations, agencies, or institutions that conduct pathogen surveillance, disease monitoring, or public health tracking activities separate from the AI-based system 102, including government health agencies, international health organizations, research institutions, clinical laboratories, veterinary surveillance networks, and environmental monitoring systems. The exchange data means the bidirectional transfer, sharing, or communication of information between different surveillance systems, involving the systematic sending and receiving of analytical results, molecular data, pathogen identification information, outbreak alerts, biological status assessments, and other relevant surveillance intelligence to enhance collective monitoring capabilities and improve coordinated response efforts.

The process of integrating the AI-based system 102 with the one or more external surveillance systems to exchange data, is implemented through multi-system interoperability frameworks that establish secure, standardized communication protocols enabling seamless information sharing between different surveillance platforms while maintaining data integrity, security, and analytical independence. This system integrating subsystem 218 operates through multiple sophisticated components beginning with data standardization and protocol harmonization modules that ensure information from the AI-based system 102 can be effectively communicated to external systems by converting proprietary data formats into standardized formats such as HL7 FHIR for clinical data, FAIR data principles for research information, and WHO surveillance reporting standards for public health data, while simultaneously developing translation algorithms that can interpret incoming data from external systems with different data structures, measurement units, and analytical methodologies. The integration framework employs Secure Communication Networks that establish encrypted, authenticated data transmission channels between the AI-based system 102 and external surveillance platforms using advanced cybersecurity protocols including end-to-end encryption, multi-factor authentication, and blockchain-based data integrity verification to ensure sensitive biological and health information remains protected during transmission while maintaining audit trails that document all data exchanges for regulatory compliance and security monitoring. The system integrating subsystem 218 utilizes real-time data synchronization algorithms that enable continuous, automated exchange of data by implementing push-pull mechanisms where the AI-based system 102 automatically transmits critical findings such as novel pathogen identifications, pandemic threat scores, and biological status assessments to relevant external systems while simultaneously receiving updates on outbreak developments, pathogen mutations, and epidemiological trends from partner surveillance networks, using intelligent filtering and prioritization algorithms that ensure only relevant, actionable information is shared to prevent data overload and maintain system efficiency. The integration framework incorporates collaborative analysis and decision support systems that enable joint analytical capabilities where the AI-based system 102 can leverage external data to enhance its own analytical accuracy while contributing its unique multianalyte analysis capabilities to improve the overall surveillance network's pathogen detection and risk assessment capabilities, using federated learning approaches that allow multiple surveillance systems to collectively improve their AI models without directly sharing sensitive raw data. Additionally, the system integrating subsystem 218 features emergency response coordination mechanisms that enable rapid information sharing during outbreak situations, automatically triggering alert notifications to relevant external systems when high-threat pathogens are detected while receiving priority alerts from partner systems about emerging biological threats, using standardized emergency communication protocols that ensure critical information reaches appropriate decision-makers across multiple organizations within minutes of detection.

For example, when detecting a novel H7N9 influenza strain with pandemic threat score of 0.91, the system integrating subsystem 218 integrates the AI-based system 102 with external surveillance systems by first establishing secure communication links with the CDC's FluView surveillance network, WHO's Global Influenza Surveillance and Response System (GISRS), and regional veterinary surveillance networks, then exchanging data by automatically transmitting the AI system's analytical results including the novel H7N9 genetic sequences, pandemic threat assessment of 0.91, human infection severity prediction of 0.89, and recommended containment measures to the CDC within 15 minutes of detection, while simultaneously receiving real-time updates from WHO GISRS showing similar H7N9 detections in three other countries with genetic similarity scores of 0.94-0.97, and obtaining veterinary surveillance data from partner systems indicating widespread H7N9 circulation in poultry populations across Southeast

Asia with mortality rates of 85-95%, enabling the integrated surveillance network to rapidly establish that this represents a coordinated multinational outbreak requiring immediate international response, where the AI system contributes its unique multianalyte pandemic threat scoring and biological status predictions while benefiting from the broader geographic surveillance coverage and epidemiological expertise of external partners, ultimately facilitating coordinated response actions including synchronized vaccine development efforts, harmonized travel restrictions, and unified public health messaging across multiple countries and organizations, thereby demonstrating how integration with external surveillance systems and systematic data exchange creates a comprehensive global surveillance network that combines the AI system's advanced analytical capabilities with the extensive monitoring reach and institutional expertise of established public health organizations to provide enhanced pathogen detection, risk assessment, and outbreak response capabilities that exceed what any single surveillance system could achieve independently.

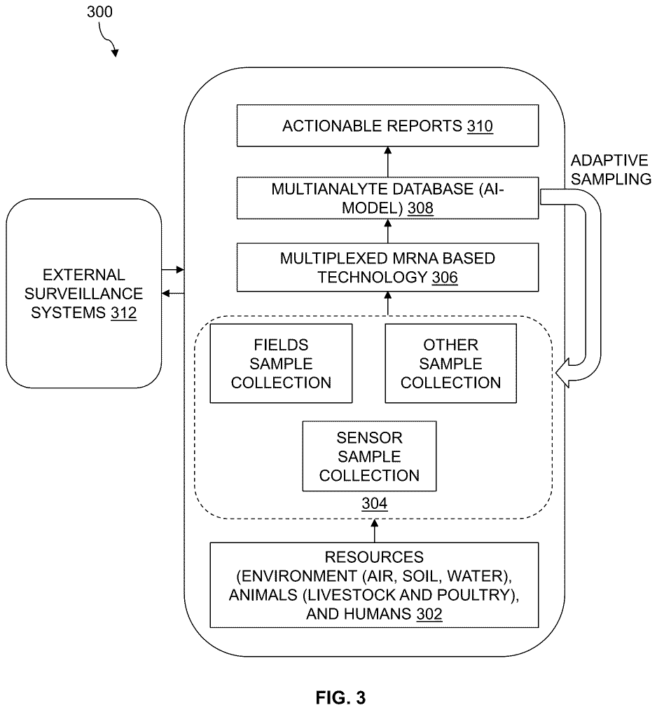

FIG. 3 illustrates an exemplary flow diagram 300 representation of a method for automatically monitoring the one or more pathogens using the multianalyte database 104, according to an example embodiment of the present disclosure. At step 302, the one or more samples are collected from the one or more resources including at least one of: the environments (e.g., air, soil, water, and the like), humans, and animals (e.g., livestock wildlife, pets,). At step 304, the analyte data are obtained from the one or more samples through at least one of: the one or more sensors, the one or more detectors, manual and automated data collectors, and laboratory analysis. At step 306, the obtained analyte data are analyzed to determine the biological status information indicative of the response of the one or more resources to each of the one or more pathogens, using the AI model (as explained in FIG. 2), as shown in step 308.

At step 310, the one or more actionable reports are generated based on the analyzed analyte data with the one or more analytes. In an embodiment, the one or more actionable reports are one or more suggestions on at least one of: the decisions on health of the one or more resources, the creation of one or more vaccines, the one or more therapeutics, supporting of the one or more research activities, and the like.

At step 312, the AI-based system 102 is integrated with the one or more external surveillance systems to exchange the data to determine the biological status information indicative of the response of the one or more resources to each of the one or more pathogens.

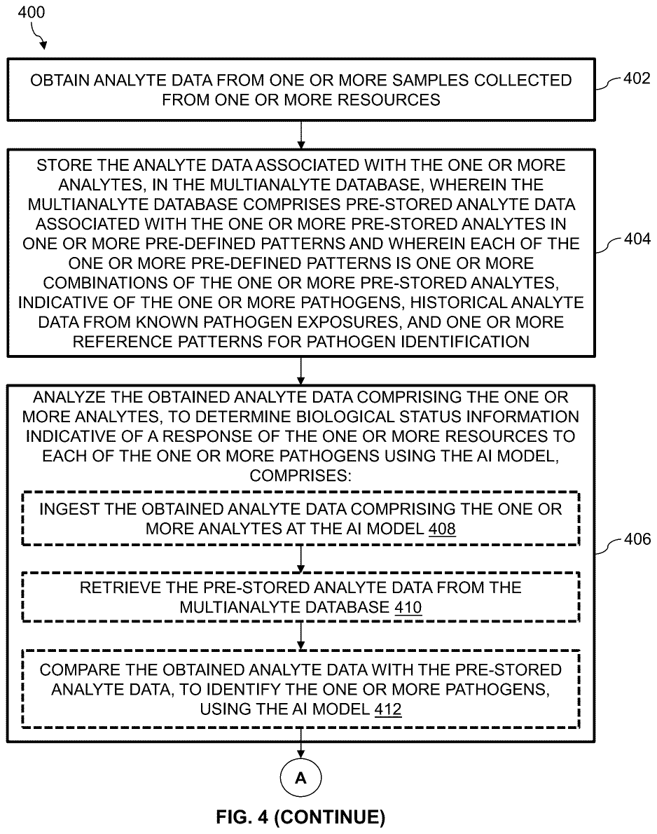

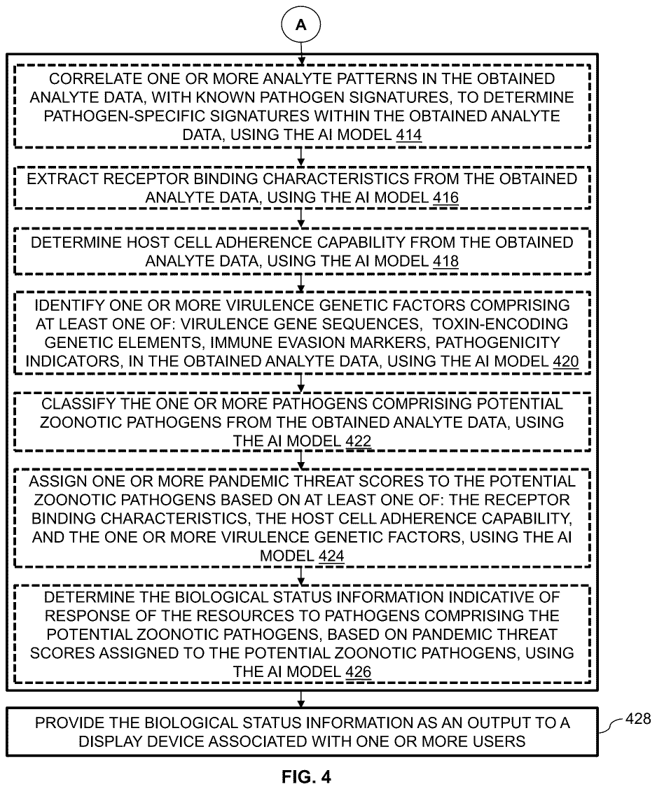

FIG. 4 illustrates a flow chart depicting an AI-based method 400 for automatically determining the biological status information of the one or more resources based on the one or more pathogens using the multianalyte database 104, according to an example embodiment of the present disclosure.

At step 402, the analyte data are obtained from the one or more samples collected from the one or more resources. In an embodiment, the one or more samples are collected from the one or more resources including at least one of: the environments (e.g., air, soil, water, and the like), the humans, and the animals (e.g., livestock animals, poultry, and the like). In an embodiment, the analyte data are obtained from the one or more samples through at least one of: the one or more sensors, the one or more detectors, manual and automated data collectors, and laboratory analysis. In an embodiment, the analyte data may include the information associated with the one or more analytes obtained from the one or more resources via the one or more samples of the one or more resources.

At step 404, the analyte data associated with the one or more analytes, are stored in the multianalyte database 104. The multianalyte database 104 may include the pre-stored analyte data associated with the one or more pre-stored analytes in the one or more pre-defined patterns. Each of the one or more pre-defined patterns is one or more combinations of the one or more pre-stored analytes, indicative of the one or more pathogens, the historical analyte data from the known pathogen exposures, and the one or more reference patterns for pathogen identification.

At step 406, the obtained analyte data comprising the one or more analytes, are analyzed to determine the biological status information indicative of the response of the one or more resources to each of the one or more pathogens using the AI model. For analyzing the obtained analyte data, the obtained analyte data comprising the one or more analytes are ingested at the AI model, as shown in step 408. Further, the pre-stored analyte data is retrieved from the multianalyte database 104, as shown in step 410. Further, the obtained analyte data is compared with the pre-stored analyte data, to identify the one or more pathogens, using the AI model, as shown in step 412. Further, the one or more analyte patterns in the obtained analyte data, is correlated with the known pathogen signatures, to determine the pathogen-specific signatures within the obtained analyte data, using the AI model, as shown in step 414.

Further, the receptor binding characteristics are extracted from the obtained analyte data, using the AI model, as shown in step 416, by: (a) identifying the receptor binding proteins and concentrations of the receptor binding proteins, within the obtained analyte data; (b) analyzing the three dimensional protein structures in the obtained analyte data for determining the binding domain compatibility; (c) evaluating the ligand receptor binding profiles present in the obtained analyte data; and (d) generating the one or more scores for binding affinity potential based on the molecular interaction data within the obtained analyte data.

Further, the host cell adherence capability are determined from the obtained analyte data, using the AI model, as shown in step 418, by: (a) detecting the adhesion protein markers and concentrations in the obtained analyte data; (b) analyzing the protein-protein interaction profiles within the obtained analyte data for the cell surface binding; (c) identifying the attachment mechanism indicators present in the obtained analyte data; and (d) quantifying the potential host cell adherence using the molecular signatures found in the obtained analyte data.

Further, the one or more virulence genetic factors comprising at least one of: virulence gene sequences, toxin-encoding genetic elements, immune evasion markers, pathogenicity indicators, are identified in the obtained analyte data, using the AI model, as shown in step 420.

Further, the one or more pathogens comprising the potential zoonotic pathogens from the obtained analyte data, are classified using the AI model, as shown in step 422, by: (a) comparing the pathogen-specific signatures within the obtained analyte data against the known zoonotic pathogen patterns; (b) identifying the species-jumping genetic markers within the obtained analyte data; (c) detecting the cross-species transmission indicators present in the obtained analyte data; and (d) classifying the potential zoonotic pathogens based on the one or more patterns associated with the potential zoonotic pathogens determined in the obtained analyte data.

Further, the one or more pandemic threat scores are assigned to the potential zoonotic pathogens based on at least one of: the receptor binding characteristics, the host cell adherence capability, and the one or more virulence genetic factors, using the AI model, as shown in step 424.

Further, the biological status information indicative of the response of the one or more resources to each of the one or more pathogens comprising the potential zoonotic pathogens, is determined based on the one or more pandemic threat scores assigned to the potential zoonotic pathogens, using the AI model, as shown in step 426.

At step 428, the biological status information indicative of the response of the one or more resources to each of the one or more pathogens comprising the potential zoonotic pathogens, is provided as an output to the display device (e.g., the user device 106) associated with one or more users.

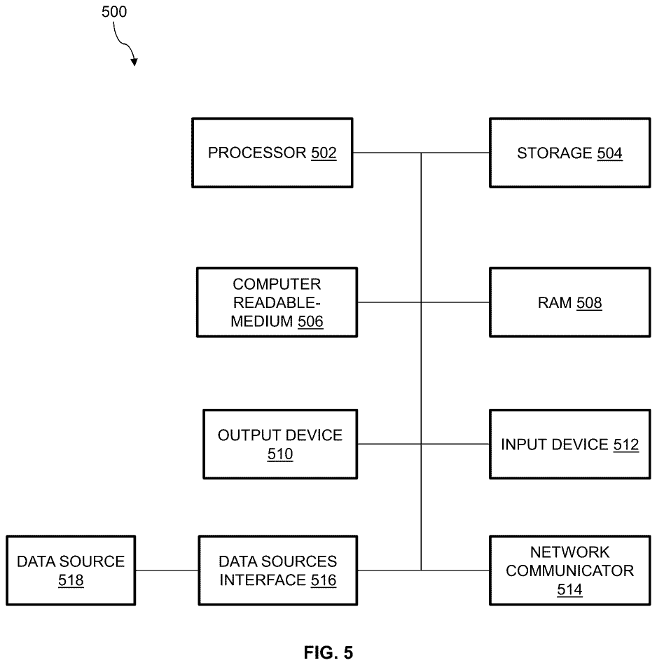

FIG. 5 illustrates an exemplary block diagram representation of a hardware platform 500 for implementation of the disclosed AI-based system 102, according to an example embodiment of the present disclosure. For the sake of brevity, the construction, and operational features of the AI-based system 102 which are explained in detail above are not explained in detail herein. Particularly, computing machines such as but not limited to internal/external server clusters, quantum computers, desktops, laptops, smartphones, tablets, and wearables which may be used to execute the AI-based system 102 or may include the structure of the hardware platform 500. As illustrated, the hardware platform 500 may include additional components not shown, and some of the components described may be removed and/or modified. For example, a computer system with multiple GPUs may be located on external-cloud platforms including Amazon Web Services, or internal corporate cloud computing clusters, or organizational computing resources.

The hardware platform 500 may be a computer system such as the system 102 (i.e., the AI-based system 102) that may be used with the embodiments described herein. The computer system may represent a computational platform that includes components that may be in a server or another computer system. The computer system may execute, by the processor 502 (e.g., single, or multiple processors) or other hardware processing circuits, the methods, functions, and other processes described herein. These methods, functions, and other processes may be embodied as machine-readable instructions stored on a computer-readable medium, which may be non-transitory, such as hardware storage devices (e.g., RAM (random access memory), ROM (read-only memory), EPROM (erasable, programmable ROM), EEPROM (electrically erasable, programmable ROM), hard drives, and flash memory). The computer system may include the processor 502 that executes software instructions or code stored on a non-transitory computer-readable storage medium 506 to perform methods of the present disclosure. The software code includes, for example, instructions to gather data and analyze the data. For example, the plurality of subsystems 118 includes the data obtaining subsystem 206, the data storage subsystem 208, the data analyzing subsystem 210, the output subsystem 212, the updating subsystem 214, the report generating subsystem 216, the information generating subsystem, and the system integrating subsystem 218.

The instructions on the computer-readable storage medium 506 are read and stored the instructions in storage 504 or random-access memory (RAM). The storage 504 may provide a space for keeping static data where at least some instructions could be stored for later execution. The stored instructions may be further compiled to generate other representations of the instructions and dynamically stored in the RAM such as RAM 508. The processor 502 may read instructions from the RAM 508 and perform actions as instructed.

The computer system may further include the output device 510 to provide at least some of the results of the execution as output including, but not limited to, visual information to users, such as external agents. The output device 510 may include a display on computing devices and virtual reality glasses. For example, the display may be a mobile phone screen or a laptop screen. GUIs and/or text may be presented as an output on the display screen. The computer system may further include an input device 512 to provide a user or another device with mechanisms for entering data and/or otherwise interacting with the computer system. The input device 512 may include, for example, a keyboard, a keypad, a mouse, or a touchscreen. Each of these output devices 510 and input device 512 may be joined by one or more additional peripherals. For example, the output device 510 may be used to display the results such as bot responses by the executable chatbot.

A network communicator 514 may be provided to connect the computer system to a network and in turn to other devices connected to the network including other clients, servers, data stores, and interfaces, for example. A network communicator 514 may include, for example, a network adapter such as a LAN adapter or a wireless adapter. The computer system may include a data sources interface 516 to access the data source 518. The data source 518 may be an information resource. As an example, a database of exceptions and rules may be provided as the data source 518. Moreover, knowledge repositories and curated data may be other examples of the data source 518.

The present invention has following advantages. The present invention provides the AI-based system 102 and the AI-based method 400, for automatically monitoring the one or more pathogens using the multianalyte database 104. The present invention may be used on the one or more analytes obtained from the samples of the one or more resources, for determining an abnormal medical pathology.

The present invention with the AI-based system 102, is configured to predict diseases before the diseases reach pandemic level and create the one or more actionable reports that can be used to guide public health decisions, create vaccines and therapeutics, and benefit research in general. The AI-based system 102 is configured to demonstrate that data ecosystems are faster than biology when it comes pandemic preemption and as such becomes a unique way or responding to emerging pandemic risks.

The present invention with the AI-based system 102 may utilize the same analyte data to: make the training process most efficient, minimize hallucinations, make a time-to-working optimized model, consume less computational power, environmental impact, and improve adaptive sampling. The present invention with the AI-based system 102 is configured to compute an immunological gap with the public based on human receptor docking and Proteomics analysis, as such assigning a risk scores based on quantitative analysis.

The active surveillance system is vertically integrated and utilizes a proprietary sample collection method, a proprietary multiplexed miRNA extraction method, and generates the multianalyte database including animal and human data. The vertically integrated active surveillance system may be fast and effective because the vertically integrated active surveillance system is made of parts that interact with each other implementing adaptive sampling and leads to minimum AI model optimization, minimum computational power consumption and environmental impact.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer-readable medium can be any apparatus that can comprise, store, communicate, propagate, or transport the program for use by or in connection with the instruction execution system, apparatus, or device.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limited, of the scope of the invention, which is outlined in the following claims.

What is claimed is:

1. An artificial intelligence (AI) based method for automatically determining biological status information of one or more resources based on one or more pathogens using multianalyte database, the AI-based method comprising:
obtaining, by one or more hardware processors, analyte data from one or more samples collected from the one or more resources, wherein the one or more samples are collected from one or more resources comprising at least one of: environments, humans and animals,
wherein the analyte data are obtained from the one or more samples through at least one of: one or more sensors, one or more detectors, manual and automated data collectors, and laboratory analysis and
wherein the analyte data comprises information associated with one or more analytes obtained from the one or more resources via the one or more samples of the one or more resources;
storing, by the one or more hardware processors, the analyte data associated with the one or more analytes, in the multianalyte database,
wherein the multianalyte database comprises pre-stored analyte data associated with the one or more pre-stored analytes in one or more pre-defined patterns and wherein each of the one or more pre-defined patterns is one or more combinations of the one or more pre-stored analytes, indicative of the one or more pathogens, historical analyte data from known pathogen exposures, and one or more reference patterns for pathogen identification;
analyzing, by the one or more hardware processors, the obtained analyte data comprising the one or more analytes, to determine the biological status information indicative of a response of the one or more resources to each of the one or more pathogens using the AI model, wherein analyzing the obtained analyte data comprises:
ingesting, by the one or more hardware processors, the obtained analyte data comprising the one or more analytes at the AI model, wherein the one or more analytes comprise at least one of: nucleic acid data, deoxyribonucleic acid (DNA), ribonucleic acid (RNA), messenger RNA (mRNA), and microRNA (miRNA), protein concentration, protein three dimensional (3D) structures, protein-protein interactions, ligand receptor binding, protein-protein interactions, ligand receptor binding, carbohydrates, disease state, molecules, and metadata, and wherein the metadata comprise at least one of: one or more species, one or more locations where the one or more samples are collected from the one or more resources, and one or more dates on which the one or more samples are collected;
retrieving, by the one or more hardware processors, the pre-stored analyte data from the multianalyte database;
comparing, by the one or more hardware processors, the obtained analyte data with the pre-stored analyte data, to identify the one or more pathogens, using the AI model;
correlating, by the one or more hardware processors, one or more analyte patterns in the obtained analyte data, with known pathogen signatures, to determine pathogen-specific signatures within the obtained analyte data, using the AI model;
extracting, by the one or more hardware processors, receptor binding characteristics from the obtained analyte data, using the AI model, by:
identifying, by the one or more hardware processors, receptor binding proteins and concentrations of the receptor binding proteins, within the obtained analyte data;
analyzing, by the one or more hardware processors, three dimensional protein structures in the obtained analyte data for determining binding domain compatibility;
evaluating, by the one or more hardware processors, ligand receptor binding profiles present in the obtained analyte data; and
generating, by the one or more hardware processors, one or more scores for binding affinity potential based on molecular interaction data within the obtained analyte data;
determining, by the one or more hardware processors, host cell adherence capability from the obtained analyte data, using the AI model, by:
detecting, by the one or more hardware processors, adhesion protein markers and concentrations in the obtained analyte data;
analyzing, by the one or more hardware processors, protein-protein interaction profiles within the obtained analyte data for cell surface binding;
identifying, by the one or more hardware processors, attachment mechanism indicators present in the obtained analyte data; and
quantifying, by the one or more hardware processors, potential host cell adherence using molecular signatures found in the obtained analyte data;
identifying, by the one or more hardware processors, one or more virulence genetic factors comprising at least one of: virulence gene sequences, toxin-encoding genetic elements, immune evasion markers, pathogenicity indicators, in the obtained analyte data, using the AI model;
classifying, by the one or more hardware processors, the one or more pathogens comprising potential zoonotic pathogens from the obtained analyte data, using the AI model, by:
comparing, by the one or more hardware processors, the pathogen-specific signatures within the obtained analyte data against known zoonotic pathogen patterns;
identifying, by the one or more hardware processors, species-jumping genetic markers within the obtained analyte data;
detecting, by the one or more hardware processors, cross-species transmission indicators present in the obtained analyte data; and
classifying, by the one or more hardware processors, the potential zoonotic pathogens based on one or more patterns associated with the potential zoonotic pathogens determined in the obtained analyte data;
assigning, by the one or more hardware processors, one or more pandemic threat scores to the potential zoonotic pathogens based on at least one of: the receptor binding characteristics, the host cell adherence capability, and the one or more virulence genetic factors, using the AI model;
determining, by the one or more hardware processors, the biological status information indicative of a response of the one or more resources to each of the one or more pathogens comprising the potential zoonotic pathogens, based on the one or more pandemic threat scores assigned to the potential zoonotic pathogens, using the AI model; and
providing, by the one or more hardware processors, the biological status information indicative of the response of the one or more resources to each of the one or more pathogens comprising the potential zoonotic pathogens, as an output to a display device associated with one or more users.

2. The AI-based method of claim 1, wherein comparing the obtained analyte data with the pre-stored analyte data, to identify the one or more pathogens, using the AI model, comprises:
matching, by the one or more hardware processors, one or more analyte patterns in the obtained analyte data against the one or more pre-defined patterns, associated with the one or more pathogens, in the pre-stored analyte data;
identifying, by the one or more hardware processors, one or more similarities and one or more differences, between current analyte profiles and historical analyte profiles;
determining, by the one or more hardware processors, correlation coefficients between the obtained analyte data and known pathogen signatures; and
determining, by the one or more hardware processors, one or more pattern matching scores for pathogen identification.

3. The AI-based method of claim 1, wherein correlating the one or more analyte patterns in the obtained analyte data, with the known pathogen signatures, to determine pathogen-specific signatures within the obtained analyte data, using the AI model, comprises:
mapping, by the one or more hardware processors, specific analyte combinations in the obtained analyte data to the known pathogen signatures;
identifying, by the one or more hardware processors, which of the one or more pathogens is potentially present based on analyte pattern matching; and
determining, by the one or more hardware processors, the pathogen-specific signatures within the obtained analyte data comprising the one or more analytes.

4. The AI-based method of claim 1, wherein assigning the one or more pandemic threat scores to the potential zoonotic pathogens, using the AI model, comprises:
integrating, by the one or more hardware processors, the receptor binding characteristics, the host cell adherence capability, and the one or more virulence genetic factors using weighted algorithmic models within the AI model to compute composite risk metrics for each of the potential zoonotic pathogens based on the obtained analyte data; and
assigning, by the one or more hardware processors, the one or more pandemic threat scores to each of the potential zoonotic pathogens by applying multi-factor risk assessment algorithms within the AI model that combine composite risk metrics derived from the receptor binding characteristics, the host cell adherence capability, and the one or more virulence genetic factors, identified in the obtained analyte data.

5. The AI-based method of claim 1, wherein determining the biological status information, using the AI model, comprises:
correlating, by the one or more hardware processors, the one or more pandemic threat scores assigned to the potential zoonotic pathogens with resource-specific response patterns stored in the multianalyte database, wherein the AI model matches high pandemic threat scores to corresponding biological response indicators for each of the one or more resources;
analyzing, by the one or more hardware processors, resource-specific vulnerability factors using the AI model by processing the obtained analyte data to identify immune system markers, stress response proteins, and inflammatory indicators specific to each of the one or more resources in response to the potential zoonotic pathogens with assigned pandemic threat scores;
generating, by the one or more hardware processors, pathogen-specific biological impact assessments using the AI model by combining the one or more pandemic threat scores with resource response data from the obtained analyte data to determine infection severity, progression rates, and recovery potential for each of the potential zoonotic pathogens affecting each of the one or more resources;
determining, by the one or more hardware processors, resource response severity levels using the AI model by weighting the one or more pandemic threat scores against resource-specific susceptibility indicators identified in the obtained analyte data, wherein higher pandemic threat scores correlate to severe biological status information for vulnerable resources; and
determining, by the one or more hardware processors, the biological status information indicative of the response of the one or more resources to each of the one or more pathogens comprising the potential zoonotic pathogens by integrating the pathogen-specific biological impact assessments and the resource response severity levels through the AI model to generate comprehensive biological status information that reflect the pandemic threat level of each potential zoonotic pathogen on each of the one or more resources.

6. The AI-based method of claim 1, further comprising generating, by the one or more hardware processors, one or more actionable reports based on the analyzed analyte data with the one or more analytes, wherein the one or more actionable reports are one or more suggestions on at least one of: decisions on health of the one or more resources, creation of one or more vaccines, one or more therapeutics, and supporting of one or more research activities.

7. The AI-based method of claim 1, further comprising dynamically adjusting, by the one or more hardware processors, the one or more pre-defined patterns associated with the one or more pre-stored analytes of the pre-stored analyte data in the multianalyte database, based on the analyte data obtained from the one or more samples of the one or more resources over a period of time, using an adaptive sampling process.

8. The AI-based method of claim 1, further comprising integrating, by the one or more hardware processors, an AI-based with one or more external surveillance systems to exchange data to determine the biological status information indicative of the response of the one or more resources to each of the one or more pathogens.

9. An artificial intelligence (AI) based system for automatically determining biological status information of one or more resources based on one or more pathogens using multianalyte database, the AI-based system comprising:
one or more hardware processors;
a memory coupled to the one or more hardware processors, wherein the memory comprises a plurality of subsystems in form of programmable instructions executable by the one or more hardware processors, and wherein the plurality of subsystems comprises:
a data obtaining subsystem configured to obtain analyte data from one or more samples collected from the one or more resources, wherein the one or more samples are collected from one or more resources comprising at least one of: environments, humans and animals,
wherein the analyte data are obtained from the one or more samples through at least one of: one or more sensors, one or more detectors, manual and automated data collectors, and laboratory analysis, and
wherein the analyte data comprises information associated with one or more analytes obtained from the one or more resources via the one or more samples of the one or more resources;
a data storage subsystem configured to store the analyte data associated with the one or more analytes, in the multianalyte database,
wherein the multianalyte database comprises pre-stored analyte data associated with the one or more pre-stored analytes in one or more pre-defined patterns and wherein each of the one or more pre-defined patterns is one or more combinations of the one or more pre-stored analytes, indicative of the one or more pathogens, historical analyte data from known pathogen exposures, and one or more reference patterns for pathogen identification;
a data analyzing subsystem configured to analyze the obtained analyte data comprising the one or more analytes, to determine the biological status information indicative of a response of the one or more resources to each of the one or more pathogens using the AI model, wherein in analyzing the obtained analyte data, the data analyzing subsystem configured to:
ingest the obtained analyte data comprising the one or more analytes at the AI model, wherein the one or more analytes comprise at least one of: nucleic acid data, deoxyribonucleic acid (DNA), ribonucleic acid (RNA), messenger RNA (mRNA), and microRNA (miRNA), protein concentration, protein three dimensional (3D) structures, protein-protein interactions, ligand receptor binding, protein-protein interactions, ligand receptor binding, carbohydrates, disease state, molecules, and metadata, and wherein the metadata comprise at least one of: one or more species, one or more locations where the one or more samples are collected from the one or more resources, and one or more dates on which the one or more samples are collected;
retrieve the pre-stored analyte data from the multianalyte database;
compare the obtained analyte data with the pre-stored analyte data, to identify the one or more pathogens, using the AI model;
correlate one or more analyte patterns in the obtained analyte data, with known pathogen signatures, to determine pathogen-specific signatures within the obtained analyte data, using the AI model;
extract receptor binding characteristics from the obtained analyte data, using the AI model, by:
identifying receptor binding proteins and concentrations of the receptor binding proteins, within the obtained analyte data;
analyzing three dimensional protein structures in the obtained analyte data for determining binding domain compatibility;
evaluating ligand receptor binding profiles present in the obtained analyte data; and
generating one or more scores for binding affinity potential based on molecular interaction data within the obtained analyte data;
determine host cell adherence capability from the obtained analyte data, using the AI model, by:
detecting adhesion protein markers and concentrations in the obtained analyte data;
analyzing protein-protein interaction profiles within the obtained analyte data for cell surface binding;
identifying attachment mechanism indicators present in the obtained analyte data; and
quantifying potential host cell adherence using molecular signatures found in the obtained analyte data;
identify one or more virulence genetic factors comprising at least one of: virulence gene sequences, toxin-encoding genetic elements, immune evasion markers, pathogenicity indicators, in the obtained analyte data, using the AI model;
classify the one or more pathogens comprising potential zoonotic pathogens from the obtained analyte data, using the AI model, by:
comparing the pathogen-specific signatures within the obtained analyte data against known zoonotic pathogen patterns;
identifying species-jumping genetic markers within the obtained analyte data;
detecting cross-species transmission indicators present in the obtained analyte data; and
classifying the potential zoonotic pathogens based on one or more patterns associated with the potential zoonotic pathogens determined in the obtained analyte data;
assign one or more pandemic threat scores to the potential zoonotic pathogens based on at least one of: the receptor binding characteristics, the host cell adherence capability, and the one or more virulence genetic factors, using the AI model; and
determine the biological status information indicative of a response of the one or more resources to each of the one or more pathogens comprising the potential zoonotic pathogens, based on the one or more pandemic threat scores assigned to the potential zoonotic pathogens, using the AI model; and
an output subsystem configured to provide the biological status information indicative of the response of the one or more resources to each of the one or more pathogens comprising the potential zoonotic pathogens, as an output to a display device associated with one or more users.

10. The AI-based system of claim 9, wherein in comparing the obtained analyte data with the pre-stored analyte data, to identify the one or more pathogens, using the AI model, the data analyzing subsystem is further configured to:
match one or more analyte patterns in the obtained analyte data against the one or more pre-defined patterns, associated with the one or more pathogens, in the pre-stored analyte data;
identify one or more similarities and one or more differences, between current analyte profiles and historical analyte profiles;
determine correlation coefficients between the obtained analyte data and known pathogen signatures; and
determine one or more pattern matching scores for pathogen identification.

11. The AI-based system of claim 9, wherein in correlating the one or more analyte patterns in the obtained analyte data, with the known pathogen signatures, to determine pathogen-specific signatures within the obtained analyte data, using the AI model, the data analyzing subsystem is further configured to:
map specific analyte combinations in the obtained analyte data to the known pathogen signatures;
identify which of the one or more pathogens is potentially present based on analyte pattern matching; and
determine the pathogen-specific signatures within the obtained analyte data comprising the one or more analytes.

12. The AI-based system of claim 9, wherein in assigning the one or more pandemic threat scores to the potential zoonotic pathogens, using the AI model, the data analyzing subsystem is further configured to:
integrate the receptor binding characteristics, the host cell adherence capability, and the one or more virulence genetic factors using weighted algorithmic models within the AI model to compute composite risk metrics for each of the potential zoonotic pathogens based on the obtained analyte data; and
assign the one or more pandemic threat scores to each of the potential zoonotic pathogens by applying multi-factor risk assessment algorithms within the AI model that combine composite risk metrics derived from the receptor binding characteristics, the host cell adherence capability, and the one or more virulence genetic factors, identified in the obtained analyte data.

13. The AI-based system of claim 9, wherein determining the biological status information, using the AI model, the data analyzing subsystem is further configured to:
correlate the one or more pandemic threat scores assigned to the potential zoonotic pathogens with resource-specific response patterns stored in the multianalyte database, wherein the AI model matches high pandemic threat scores to corresponding biological response indicators for each of the one or more resources;
analyze resource-specific vulnerability factors using the AI model by processing the obtained analyte data to identify immune system markers, stress response proteins, and inflammatory indicators specific to each of the one or more resources in response to the potential zoonotic pathogens with assigned pandemic threat scores;
generate pathogen-specific biological impact assessments using the AI model by combining the one or more pandemic threat scores with resource response data from the obtained analyte data to determine infection severity, progression rates, and recovery potential for each of the potential zoonotic pathogens affecting each of the one or more resources;
determine resource response severity levels using the AI model by weighting the one or more pandemic threat scores against resource-specific susceptibility indicators identified in the obtained analyte data, wherein higher pandemic threat scores correlate to severe biological status information for vulnerable resources; and
determine the biological status information indicative of the response of the one or more resources to each of the one or more pathogens comprising the potential zoonotic pathogens by integrating the pathogen-specific biological impact assessments and the resource response severity levels through the AI model to generate comprehensive biological status information that reflect the pandemic threat level of each potential zoonotic pathogen on each of the one or more resources.

14. The AI-based system of claim 9, further comprising a report generating subsystem configured to generate one or more actionable reports based on the analyzed analyte data with the one or more analytes, wherein the one or more actionable reports are one or more suggestions on at least one of: decisions on health of the one or more resources, creation of one or more vaccines, one or more therapeutics, and supporting of one or more research activities.

15. The AI-based system of claim 9, further comprising an updating subsystem is configured to dynamically adjust the one or more pre-defined patterns associated with the one or more pre-stored analytes of the pre-stored analyte data in the multianalyte database, based on the analyte data obtained from the one or more samples of the one or more resources over a period of time, using an adaptive sampling process.

16. The AI-based system of claim 9, further comprising a system integrating subsystem configured to integrate the AI-based system with one or more external surveillance systems to exchange data to determine the biological status information indicative of the response of the one or more resources to each of the one or more pathogens.

17. A non-transitory computer-readable storage medium having instructions stored therein that when executed by one or more hardware processors, cause the one or more hardware processors to execute operations of:
obtaining analyte data from one or more samples collected from one or more resources, wherein the one or more samples are collected from one or more resources comprising at least one of: environments, humans and animals,
wherein the analyte data are obtained from the one or more samples through at least one of: one or more sensors, one or more detectors, manual and automated data collectors, and laboratory analysis, and
wherein the analyte data comprises information associated with one or more analytes obtained from the one or more resources via the one or more samples of the one or more resources;
storing the analyte data associated with the one or more analytes, in the multianalyte database,
wherein the multianalyte database comprises pre-stored analyte data associated with the one or more pre-stored analytes in one or more pre-defined patterns and wherein each of the one or more pre-defined patterns is one or more combinations of the one or more pre-stored analytes, indicative of one or more pathogens, historical analyte data from known pathogen exposures, and one or more reference patterns for pathogen identification;
analyzing the obtained analyte data comprising the one or more analytes, to determine biological status information indicative of a response of the one or more resources to each of the one or more pathogens using the AI model, wherein analyzing the obtained analyte data comprises:
ingesting the obtained analyte data comprising the one or more analytes at the AI model, wherein the one or more analytes comprise at least one of: nucleic acid data, deoxyribonucleic acid (DNA), ribonucleic acid (RNA), messenger RNA (mRNA), and microRNA (miRNA), protein concentration, protein three dimensional (3D) structures, protein-protein interactions, ligand receptor binding, protein-protein interactions, ligand receptor binding, carbohydrates, disease state, molecules, and metadata, and wherein the metadata comprise at least one of: one or more species, one or more locations where the one or more samples are collected from the one or more resources, and one or more dates on which the one or more samples are collected;
retrieving the pre-stored analyte data from the multianalyte database;
comparing the obtained analyte data with the pre-stored analyte data, to identify the one or more pathogens, using the AI model;
correlating one or more analyte patterns in the obtained analyte data, with known pathogen signatures, to determine pathogen-specific signatures within the obtained analyte data, using the AI model;
extracting receptor binding characteristics from the obtained analyte data, using the AI model, by:
identifying receptor binding proteins and concentrations of the receptor binding proteins, within the obtained analyte data;
analyzing three dimensional protein structures in the obtained analyte data for determining binding domain compatibility;
evaluating ligand receptor binding profiles present in the obtained analyte data; and
generating one or more scores for binding affinity potential based on molecular interaction data within the obtained analyte data;
determining host cell adherence capability from the obtained analyte data, using the AI model, by:
detecting adhesion protein markers and concentrations in the obtained analyte data;
analyzing protein-protein interaction profiles within the obtained analyte data for cell surface binding;
identifying attachment mechanism indicators present in the obtained analyte data; and
quantifying potential host cell adherence using molecular signatures found in the obtained analyte data;
identifying one or more virulence genetic factors comprising at least one of:
virulence gene sequences, toxin-encoding genetic elements, immune evasion markers, pathogenicity indicators, in the obtained analyte data, using the AI model;
classifying the one or more pathogens comprising potential zoonotic pathogens from the obtained analyte data, using the AI model, by:
comparing the pathogen-specific signatures within the obtained analyte data against known zoonotic pathogen patterns;
identifying species-jumping genetic markers within the obtained analyte data;
detecting cross-species transmission indicators present in the obtained analyte data; and
classifying the potential zoonotic pathogens based on one or more patterns associated with the potential zoonotic pathogens determined in the obtained analyte data;
assigning one or more pandemic threat scores to the potential zoonotic pathogens based on at least one of: the receptor binding characteristics, the host cell adherence capability, and the one or more virulence genetic factors, using the AI model;
determining the biological status information indicative of a response of the one or more resources to each of the one or more pathogens comprising the potential zoonotic pathogens, based on the one or more pandemic threat scores assigned to the potential zoonotic pathogens, using the AI model; and
providing the biological status information indicative of the response of the one or more resources to each of the one or more pathogens comprising the potential zoonotic pathogens, as an output to a display device associated with one or more users.

18. The non-transitory computer-readable storage medium of claim 17, wherein comparing the obtained analyte data with the pre-stored analyte data, to identify the one or more pathogens, using the AI model, comprises:
matching one or more analyte patterns in the obtained analyte data against the one or more pre-defined patterns, associated with the one or more pathogens, in the pre-stored analyte data;
identifying one or more similarities and one or more differences, between current analyte profiles and historical analyte profiles;
determining correlation coefficients between the obtained analyte data and known pathogen signatures; and
determining one or more pattern matching scores for pathogen identification.

19. The non-transitory computer-readable storage medium of claim 17, wherein correlating the one or more analyte patterns in the obtained analyte data, with the known pathogen signatures, to determine pathogen-specific signatures within the obtained analyte data, using the AI model, comprises:
mapping specific analyte combinations in the obtained analyte data to the known pathogen signatures;
identifying which of the one or more pathogens is potentially present based on analyte pattern matching; and
determining the pathogen-specific signatures within the obtained analyte data comprising the one or more analytes.

20. The non-transitory computer-readable storage medium of claim 17, wherein determining the biological status information, using the AI model, comprises:
correlating the one or more pandemic threat scores assigned to the potential zoonotic pathogens with resource-specific response patterns stored in the multianalyte database, wherein the AI model matches high pandemic threat scores to corresponding biological response indicators for each of the one or more resources;
analyzing resource-specific vulnerability factors using the AI model by processing the obtained analyte data to identify immune system markers, stress response proteins, and inflammatory indicators specific to each of the one or more resources in response to the potential zoonotic pathogens with assigned pandemic threat scores;
generating pathogen-specific biological impact assessments using the AI model by combining the one or more pandemic threat scores with resource response data from the obtained analyte data to determine infection severity, progression rates, and recovery potential for each of the potential zoonotic pathogens affecting each of the one or more resources;
determining resource response severity levels using the AI model by weighting the one or more pandemic threat scores against resource-specific susceptibility indicators identified in the obtained analyte data, wherein higher pandemic threat scores correlate to severe biological status information for vulnerable resources; and
determining the biological status information indicative of the response of the one or more resources to each of the one or more pathogens comprising the potential zoonotic pathogens by integrating the pathogen-specific biological impact assessments and the resource response severity levels through the AI model to generate comprehensive biological status information that reflect the pandemic threat level of each potential zoonotic pathogen on each of the one or more resources.
